(19) United States
(12) Patent Application Publication
(10) Pub. No.: US 2025/0361500 A1
(43) Pub. Date: Nov. 27, 2025

(54) (S)-ENGINEERED OXYNITRILASE POLYPEPTIDES AND USES THEREOF

(71) Applicant: Novartis AG, Basel (CH)

(72) Inventors: Oscar ALVIZO, Redwood City, CA (US); Michael Umberto LUESCHER, Zurich (CH); Gregory MANN, Basel (CH); Benjamin MARTIN, Leymen (FR); Scott J. NOVICK, Redwood City, CA (US); Ruairi Seosamh O MEADHRA, Sierentz (FR); Theo PESCHKE, Weil am Rhein (DE); Thierry SCHLAMA, Buschwiller (FR); Lukas Christian SCHOBER, Weil am Rhein (DE); Kirsten SCHROER, Zeiningen (DE); Frederic Valentin STANGER, Colmar (FR); Nandhitha SUBRAMANIAN, Cambridge (GB); Nhat Quang Nguyen TRUNG, Galfingue (FR)

(21) Appl. No.: 18/872,024

(22) PCT Filed: Jun. 8, 2023
(86) PCT No.: PCT/IB2023/055923

§ 371 Date: Dec. 5, 2024

Related U.S. Application Data
(60) Provisional application No. 63/350,047, filed on Jun. 8, 2022.

(51) Int. Cl. C12N 9/88; C07C 213/02; C07D 213/04; C12P 13/00

(52) U.S. Cl. CPC C12N 9/88; C07C 213/02; C07D 213/04; C12P 13/008

(57) ABSTRACT

The present disclosure relates to a process for producing chiral β-nitro alcohol compounds. The invention relates in particular to an (S)-selective oxynitrilase, which enantioselectively can catalyze the Henry reaction, wherein an aldehyde or ketone compound is converted to the corresponding β-nitro alcohol compound in the presence of a nitroalkane compound and an oxynitrilase.

### CLAIM OF PRIORITY

This application claims the benefit of priority to U.S. Provisional Application No. 63/350,047 filed Jun. 8, 2022, the disclosure of which is incorporated by reference herein in its entirety.

### SEQUENCE LISTING

The instant application contains a Sequence Listing which has been submitted electronically in ST.26 format and is hereby incorporated by reference in its entirety. Said ST.26 copy, created on Jun. 7, 2023 is named PAT059273-WO-PCT_SL.xml.

### TECHNICAL FIELD

The present disclosure relates to the field of biotechnology, in particular to engineered ox ynitrilase polypeptides and their application in industrial biocatalysis. In particular, the present disclosure relates to a method for producing chiral 3-nitro alcohol compounds, wherein an aldehyde or ketone compound is converted to the corresponding β-nitro alcohol compound in the presence of a nitroalkane compound and an engineered oxynitrilase. The disclosure relates in particular to an (S)-selective oxynitrilase which enantioselectively catalyzes the Henry reaction.

### BACKGROUND ART

Biocatalytic processes have become very important to the chemical industry. Of particular importance is the use of enzymes, when the properties of biocatalysts enable either of the two enantiomers in chemical reactions with chiral or prochiral compounds to be reacted or formed preferentially.

Essential requirements for utilizing these favorable properties of enzymes are their low-cost availability in sufficient amounts, as required in industrial processes, a sufficiently high reactivity, selectivity and high stability under the realistic conditions of the industrial process.

β-nitro alcohols are precursors for β-amino alcohols, which are important chiral building blocks for the synthesis of bioactive compounds. The nitroaldol or Henry reaction is one of the classical named reactions in organic synthesis for C—C bond formation. Due to the potential to create up to two new chiral centers it is of fundamental importance for synthetic applications to be able to perform the nitroaldol addition enantio- and stereoselectively. Although the reaction has been known for more than a century (Henry, 1895), stereospecific protocols utilizing non-enzymatic organocatalysts or chiral metal catalysts have been developed only recently. The development of these methods still share a number of disadvantages, including long reaction times and sometimes extreme reaction conditions in the case of metal catalysts, or insufficient selectivities in the case of organocatalysts.

Hydroxynitrile Lyases (HNLs), often also called Oxynitrilases, belong to the enzyme class of aldehyde lyases (E.C. 4.1.2.X). In nature HNLs catalyze the reversible stereoselective cleavage of hydroxy nitriles into hydrocyanic acids and aldehydes or ketones. This cyanogenesis reaction is utilized by plants to defend against fungi or predators by releasing hydrogen cyanide in the cells. In reversal of their natural reaction, HNLs also catalyze the stereoselective addition of hydrocyanic acid to aldehydes or ketones to yield enantiopure hydroxy nitriles, which are often utilized as building blocks for various pharmaceuticals and agrochemicals (Dadashipour & Asano ACS Catalysis 2011 1 (9), 1121-1149). For the nitrile formation HNLs have shown limited substrate scope, with respect to the nature of the electrophile accepting aliphatic and aromatic aldehydes or aliphatic ketones compounds while only cyanide is accepted as nucleophile (Liu et al. Front. Bioeng. Biotechnol. 2021 9:653682).

The HNL from Hevea brasiliensis have been the first described enzyme able to catalyze an enzymatic nitroaldol (Henry) reaction of aldehydes with nitromethane (Mandana Gruber-Khadjawi et al. Adv. Synth. Catal. 2007, 349, 1445-1450). In recent years more examples of (R)-selective HNLs catalyzing the Henry reaction originating from Acidobacterium capsulatum, Granulicella tundricula (Bekerle-Bogner et al. ChemCatChem 2016, 8, 2214) or Arabidobsis thaliana (Fuhshuku et al. J. Biotechnol. 2011, 153, 153-159) have been described. Amongst these, the latter one from Arabidobsis thaliana (AtHNL) is the most widely described (Fuhshuku et al. J. Biotechnol. 2011, 153, 153-159).

Beyond HNLs also TGase (protein-glutamine-glutamyltransferase; EC 2.3.2.13) from Streptorerticillium griseoverticillatum have been described to catalyze the Henry reaction.

Therefore, the development of asymmetric synthesis of β-nitro alcohols is in great demand. There is still the need for new oxynitrilases, which can enantioselectively catalyze the Henry reaction.

The present disclosure provides a series of engineered polypeptides with high stereoselec tivity which overcomes the above-mentioned shortcomings.

### BRIEF DESCRIPTION OF THE DRAWINGS

FIG. 1A shows comparative data for the conversion of trifluoroacetone (1) and nitromethane (2) to the nitro alcohol compound (IA) with the wildtype (S)-hydroxynitrile lyase from Baliospermum montanum, Uniprot ID: D1MX73 having SEQ ID NO: 606 versus the engineered polypeptide SEQ ID NO: 2.

FIG. 1B shows comparative data for the enantiomeric excess in the reaction of trifluoroacetone (1) and nitromethane (2) to give the nitro alcohol compound (IA) with the wildtype (S)-hydroxynitrile lyase from Baliospermum montanum, Uniprot ID: D1MX73 having SEQ ID NO: 606 versus the engineered polypeptide SEQ ID NO: 2.

FIG. 2A shows comparative conversion data for the most active polypeptides from each evolution round for the reaction of trifluoroacetone (1) and nitromethane (2) to produce the nitro alcohol compound (IA), under the reaction conditions described in Example 13.

FIG. 2B shows comparative enantiomeric excess data for the most active polypeptides from each evolution round for the reaction of trifluoroacetone (1) and nitromethane (2) to produce the nitro alcohol compound (IA), under the reaction conditions described in Example 13.

FIG. 2C shows comparative conversion data for the most active polypeptides from each evolution round for the reaction of trifluoroacetone (1) and nitromethane (2) to produce the nitro alcohol compound (IA), under the reaction conditions described in Example 13.

FIG. 2D shows comparative enantiomeric excess data for the most active polypeptides from each evolution round for the reaction of trifluoroacetone (1) and nitromethane (2) to produce the nitro alcohol compound (IA), under the reaction conditions described in Example 13.

FIG. 3A shows the activity of the free enzyme compared to the immobilized enzymes on either amino or epoxy carrier.

FIG. 3B shows the selectivity of the free enzyme compared to the immobilized enzymes on either amino or epoxy carrier.

FIG. 4 shows the residue difference of the claimed oxynitrilase polypeptides relative to SEQ ID No: 606 and SEQ ID No: 2.

### SUMMARY OF THE DISCLOSURE

The present disclosure provides engineered polypeptides with high stereoselectivity, high catalytic activity and good stability, which can asymmetrically synthesize β-nitro alcohols, and in particular asymmetrically synthesize (S)-1,1,1-trifluoro-2-methyl-3-nitropropan-2-ol. The disclosure relates in particular to an (S)-selective oxynitrilase, which can enantioselectively catalyze the Henry reaction.

Surprisingly, the engineered polypeptides of the disclosure are particularly amenable to substrates comprising electron withdrawing groups. As a result, it has been found that β-nitro alcohol products can be synthesized in a high yield with high stereoselectivity by introducing an electron withdrawing substituent in the aldehyde or ketone substrate.

The present disclosure also provides gene sequences of engineered polypeptides, recombinant expression vectors comprising the genes, engineered strains and efficient methods for the production thereof, as well as reaction processes for the asymmetric synthesis of β-nitro alcohols using engineered polypeptides.

The engineered oxynitrilase polypeptides disclosed herein have improved catalytic properties. Through substitutions, insertions, or deletions of a number of amino acid residues in directed evolution processes, these engineered polypeptides were derived from a wild-type oxynitrilase which is less stereoselective towards the product. The wild-type oxynitrilase is from Baliospermum montanum (BmHNL), which consists of 263 amino acids and has the sequence shown in SEQ ID No: 606 (also accessible under accession number D1MX73 in UniProt). The wild-type oxynitrilase showed low stereoselectivity for the product. In the reaction of 1,1,1-trifluoropropan-2-one with nitromethane to produce (S)-1,1,1-trifluoro-2-methyl-3-nitropropan-2-ol (i.e., (IA)) using SEQ ID No: 606, the enantiomeric excess (i.e., ee) for IA was <2%.

In a first aspect, there is provided an oxynitrilase polypeptide, which is a polypeptide of (a) or (b) below:
(a) a polypeptide comprising an amino acid sequence selected from the group consisting of SEQ ID NOs: 4 to 604, and 608 to 640; or
(b) a polypeptide having oxynitrilase activity, which comprises an amino acid sequence having (i) at least 80% sequence identity to one of the polypeptides recited in (a), and (ii) a substitution, deletion, addition or insertion of one or more amino acid residues relative to said one amino acid sequence recited in (a).

In a second aspect, there is provided an oxynitrilase polypeptide, which is capable of coupling 1,1,1-trifluoropropan-2-one with nitromethane to produce (S)-1,1,1-trifluoro-2-methyl-3-nitropropan-2-ol, under suitable reaction conditions, at greater stereoselectivity and/or activity than that of SEQ ID NO: 606.

In a third aspect, there is provided an oxynitrilase polypeptide comprising an amino acid sequence having at least 80% sequence identity to SEQ ID NO: 606, which is, under suitable reaction conditions, capable of coupling 1,1,1-trifluoropropan-2-one with nitromethane to produce (S)-1,1,1-trifluoro-2-methyl-3-nitropropan-2-ol in an enantiomeric excess of at least 60%, 70%, 80%, 85%, 90%, 91%, 92%, 93%, 94%, 95%, 96%, 97%, 98%, 99% or more.

In a further aspect, there is provided a polypeptide immobilized on a solid material by chemical bond or a physical adsorption method, wherein the polypeptide is selected from the oxynitrilase polypeptides according to the disclosure.

In a further aspect, there is provided a polynucleotide encoding a polypeptide of the disclosure.

In a further aspect, there is provided an expression vector comprising a polynucleotide according to the disclosure.

In a further aspect, there is provided a host cell comprising the expression vector of the disclosure, wherein the host cell is preferably E. coli.

In a further aspect, there is provided a method of preparing an oxynitrilase polypeptide, which comprises the steps of culturing the host cell according the disclosure and obtaining an oxynitrilase polypeptide from the culture.

In a further aspect, there is provided an oxynitrilase catalyst obtainable by culturing the host cells of the disclosure, wherein said oxynitrilase catalyst comprises cells or culture fluid containing the oxynitrilase polypeptides, or an article processed therewith, wherein the article refers to an extract obtained from the culture of transformant cell, an isolated product obtained by isolating or purifying an oxynitrilase from the extract, or an immobilized product obtained by immobilizing transformant cell, an extract thereof, or isolated product of the extract.

In a further aspect, there is provided a process for the asymmetric synthesis of a β-nitro alcohol using an oxynitrilase polypeptide, the process comprising the step of contacting a nitroalkane and an aldehyde or ketone substrate with the oxynitrilase polypeptide, to obtain a β-nitro alcohol product.

In a further aspect, there is provided a process for the asymmetric synthesis of a β-nitro alcohol using the herein disclosed engineered oxynitrilase polypeptides, the process comprising the step of contacting a nitroalkane and an aldehyde or ketone substrate with the oxynitrilase polypeptide of the present disclosure, to obtain a β-nitro alcohol product.

In a further aspect, there is provided a process a process for the asymmetric synthesis of (S)-1,1,1-trifluoro-2-methyl-3-nitropropan-2-ol (IA):

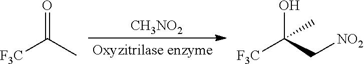

the process comprising the step of contacting nitromethane and 1,1,1-trifluoropropan-2-one with the oxynitrilase polypeptide according to the present disclosure, to obtain (S)-1,1,1-trifluoro-2-methyl-3-nitropropan-2-ol of formula (IA).

In a further aspect, there is provided a process for synthesizing (S)-3-amino-1,1,1-trifluoro-2-methylpropan-2-ol of formula (IB):

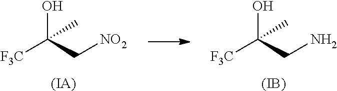

the process comprising the step of contacting (IA) with hydrogen under suitable hydrogenation conditions, to obtain (S)-3-amino-1,1,1-trifluoro-2-methylpropan-2-ol (IB), wherein (IA) is synthesized by the process according to according to the present disclosure.

In a further aspect, there is provided a process for synthesizing (S)-3-amino-6-methoxy-N-(3,3,3-trifluoro-2-hydroxy-2-methylpropyl)-5-(trifluoromethyl)picolinamide of formula (IC)

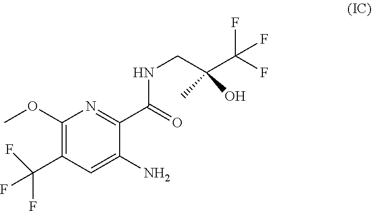 [SMILES: COc1nc(C(=O)NC[C@](C)(O)C(F)(F)F)c(N)cc1C(F)(F)F]

the process comprising the step of contacting nitromethane and 1,1,1-trifluoropropan-2-one with the oxynitrilase polypeptide according to the present disclosure, to obtain (S)-1,1,1-trifluoro-2-methyl-3-nitropropan-2-ol of formula (IA).

### DETAILED DESCRIPTION

The present disclosure describes the directed evolution of a HNL to obtain nitro alcohols in excellent yields and enantiomeric excesses even at equimolar ratios of the substrates. It is the first description of a HNL accepting ketones as substrates for a Henry reaction and also the first S-selective Oxynitrilase catalyzing the Henry reaction. The HNLs originate from the organism Baliospermum montanum (BmHNL) also never have been described to be able to catalyze the Henry reaction. A series of engineered polypeptides with high S-stereoselectivity is provided.

These engineered polypeptides were developed through directed evolution towards the se lection of (S)-1,1,1-trifluoro-2-methyl-3-nitropropan-2-ol, a compound of formula (IA) as defined herein.

The present disclosure describes engineered polypeptides originating from BmHNL, catalyzing the Henry reaction of trifluoroacetone (1) and nitromethane (2) to produce the nitro alcohol compound (IA). All so far described Oxynitrilase need large excess of the nitro compound from 10 to even 45-fold to reach decent conversion >50% leading to a poor atom efficiency (<20%) and economic feasibility of these processes while the newly described polypeptide can reach >80% conversion and >80% ee at equimolar conditions or 100% conversion and >90% ee with 1.2-fold excess of the substrate nitromethane (2). It is the first description of a HNL accepting ketones as substrates for a Henry reaction and also the first described highly S-Selective Oxynitrilase catalyzing the Henry reaction with an ee >80%.

The present disclosure also provides a process for the asymmetric synthesis of a β-nitro alcohol using the herein disclosed engineered oxynitrilase polypeptides, the process comprising the step of contacting a nitroalkane and an aldehyde or ketone substrate with the oxynitrilase polypeptide of the present disclosure, to obtain a β-nitro alcohol product.

In particular, the present disclosure provides a process for the asymmetric synthesis of (S)-1,1,1-trifluoro-2-methyl-3-nitropropan-2-ol (IA), which process stereoselectively produces the desired (S) enantiomer over the (R) enantiomer.

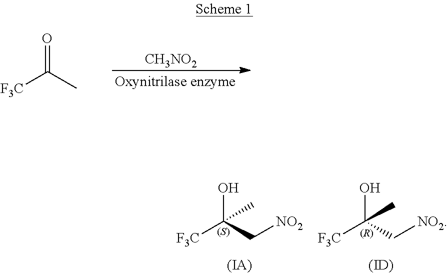

Compound (IA), i.e., (S)-1,1,1-trifluoro-2-methyl-3-nitropropan-2-ol, is an intermediate in the synthesis of

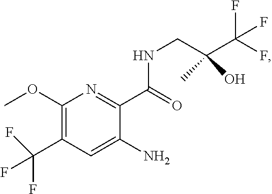 [SMILES: COc1nc(C(=O)NC[C@](C)(O)C(F)(F)F)c(N)cc1C(F)(F)F]

i.e., (S)-3-amino-6-methoxy-N-(3,3,3-trifluoro-2-hydroxy-2-methylpropyl)-5-(trifluoromethyl)picolinamide, also referred to herein as (IC), for the treatment of cystic fibrosis.

The enzymatic synthesis of compound (IA) presents an attractive alternative to traditional chemical synthesis. The use of enzyme biocatalysts may also reduce chemical waste and allow to shorten the overall number of steps required in the synthesis.

### Definitions

Unless expressly defined otherwise, technical and scientific terms used in this disclosure have the meanings that are commonly understood by people skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The abbreviations used for the genetically encoded amino acids are conventional and are as follows:

| Amino Acid | Three-Letter | One-Letter Abbreviation |
|---|---|---|
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | HIS | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

For a deletion of an amino acid a “−” was used and for a Stop Codon a “*”. When the three-letter abbreviations are used, unless specifically preceded by an “L” or a “D” or clear from the context in which the abbreviation is used, the amino acid may be in either the L- or D-configuration about α-carbon (C). For example, whereas “Ala” designates alanine without specifying the configuration about the α-carbon, “D-Ala” and “L-Ala” designate D-alanine and L-alanine, respectively.

When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

The abbreviations used for the genetically encoding nucleotides are conventional and are as follows: adenosine (A); guanosine (G); cytidine (C); thymidine (T); and uridine (U). Unless specifically delineated, the abbreviated nucleotides may be either ribonucleotides or 2′-deoxyribonucleotides. The nucleotides may be specified as being either ribonucleotides or 2′-deoxyribonucleotides on an individual basis or on an aggregate basis. When nucleic acid sequences are presented as a string of one-letter abbreviations, the sequences are presented in the 5′ to 3′ direction in accordance with common convention, and the phosphates are not indicated.

“Amino acid difference” or “residue difference” refers to the difference in amino acid residues at a position of a polypeptide sequence relative to the amino acid residue at a corresponding position in the reference sequence. The positions of amino acid differences are generally referred to herein as “Xn”, where n refers to the corresponding position in the reference sequence on which the residue differences are based. For example, “a residue difference at position X2 as compared to SEQ ID NO: 2” refers to the difference in amino acid residues at the polypeptide position corresponding to position 2 of SEQ ID NO: 2. Thus, if the reference polypeptide of SEQ ID NO: 2 has a leucine at position 2, then “a residue difference at position X2 as compared to SEQ ID NO: 2” refers to an amino acid substitution of any residue other than leucine at the position of the polypeptide corresponding to position 2 of SEQ ID NO: 2. In most of the examples herein, the specific amino acid residue difference at the position is indicated as “XnY”, wherein “Xn” specified to the corresponding position as described above, and “Y” is the single letter identifier of the amino acid found in the engineered polypeptide (i.e., a different residue than in the reference polypeptide). In some examples (e.g., in FIG. 4), the present disclosure also provides specific amino acid differences denoted by the conventional notation “AnB”, where A is a single letter identifier of a residue in the reference sequence, “n” is the number of residue position in the reference sequence, and B is the single letter identifier for the residue substitution in the sequence of the engineered polypeptide. In some examples, an engineered polypeptide of this disclosure may comprise one or more amino acid residue differences relative to a reference sequence, which is indicated by a list of specific positions at which residue differences are present relative to a reference sequence. In some embodiments, more than one amino acid residue can be used in a specific residue position of an engineered polypeptide, the various amino acid residues that can be used are separated by a “/” (e.g., X38F/X38F). “Deletion” refers to the modification of a polypeptide by removing one or more amino acids from a reference polypeptide. Deletions can include the removal of one or more amino acids, two or more amino acids, five or more amino acids, ten or more amino acids, fifteen or more amino acids, or twenty or more amino acids, up to 10% of the total number of amino acids of the enzyme, or up to 20% of the total number of amino acids making up the reference enzyme while retaining the enzymatic activity of the engineered oxynitrilase and/or retaining the improved properties of the engineered oxynitrilase. Deletion may involve the internal portion and/or the terminal portion of the polypeptide. In various embodiments, deletions may include a contiguous segment or may be discontinuous.

The term “and/or” means either “and” or “or” unless indicated otherwise.

“Coding sequence” refers to that portion of a nucleic acid (e.g., a gene) that encodes an amino acid sequence of a protein.

A “comparison window” refers to a conceptual segment of at least about 20 contiguous nucleotide positions or amino acid residues, wherein the sequence may be compared to a reference sequence of at least 20 contiguous nucleotides or amino acids and wherein the portions of the sequence in the comparison window may comprise additions or deletions (i.e., gaps) of 20% or less as compared to a reference sequence (which does not comprise additions or deletions) for optimal alignment of the two sequences. The comparison window can be longer than 20 contiguous residues, and optionally include 30, 40, 50, 100 or more residues.

Unless specified otherwise, the terms “compounds of the present disclosure,” “compounds of the disclosure,” or “compound of the disclosure” refer to compounds of formulae (1), (2), (I), (I-i), (II), (III), (IA), (IB), (IB).HCl, (IC), (ID), E1, E3, E4, E5, E6, exemplified compounds, salts thereof, particularly pharmaceutically acceptable salts thereof, hydrates, solvates, prodrugs, as well as all stereoisomers (including diastereoisomers and enantiomers), rotamers, tautomers, and isotopically labeled compounds (including deuterium substitutions), as well as inherently formed moieties.

“Conversion” refers to the enzymatic transformation of a substrate to the corresponding product. “Percent conversion” or “conversion” refers to the percentage of substrate that is converted to product within a period of time under the specified conditions. Thus, “enzymatic activity” or “activity” of an oxynitrilase polypeptide can be expressed as the “percent conversion” of the substrate to the product.

In the context of the numbering for a given amino acid or polynucleotide sequence, “corresponding to, ““reference to” or “relative to” refers to the numbering of the residues of a specified reference when the given amino acid or polynucleotide sequence is compared to the reference sequence. In other words, the residue number or residue position of a given sequence is designated with respect to the reference sequence, rather than by the actual numerical position of the residue within the given amino acid or polynucleotide sequence. For example, a given amino acid sequence such as an engineered oxynitrilase can be aligned to a reference sequence by introducing gaps to optimize residue matches between the two sequences. In these cases, although there are gaps, the numbering of the residue in a given amino acid or polynucleotide sequence is made with respect to the reference sequence to which they have been aligned.

The term “electron withdrawing substituent”, as used herein, refers to a substituent that has a strong electron withdrawing force. Exemplary electron withdrawing groups include, without limitation, halogen atoms such as fluorine atom, haloalkyl groups such as trifluoromethyl group, carboxyl group, alkoxycarbonyl groups such as methoxycarbonyl group, aryloxycarbonyl groups such as phenoxycarbonyl group, acyl groups such as acetyl group, acyloxy groups such as acetoxy group, cyano group, aryl groups, alkenyl groups, nitro group, sulfo group, alkanesulfonyl groups, alkanesulfinyl groups, and alkoxysulfonyl groups, including any of the substituents disclosed herein, such as alkyl, alkenyl, alkynyl, aryl, hetereoaryl, heterocyclyl, cycloalkyl, and arylalkyl groups containing these electron withdrawing groups as substituents. Among them, preferred examples are fluorine-containing groups such as fluorine atom and trifluoromethyl group, acyloxy groups such as acetoxy group, cyano group, nitro group, sulfo group, alkylsulfonyl groups, e.g., C₁-C₆alkylsulfonyl, alkylsulfinyl groups, e.g., C₁-C₆alkylsulfinyl, and alkoxysulfonyl groups, e.g., C₁-C₆alkoxylsulfonyl.

The terms “Engineered oxynitrilase”, “engineered oxynitrilase polypeptide”, “oxynitrilase polypeptide”, “improved oxynitrilase polypeptide”, and “engineered polypeptide” are used interchangeably herein.

“Fragment” as used herein refers to a polypeptide having an amino terminal and/or carboxyl terminal deletion, but where the remaining amino acid sequence is identical to the corresponding position in the sequence. Fragments may be at least 10 amino acids long, at least amino acids long, at least 50 amino acids long or longer, and up to 70%, 80%, 90%, 95%, 98% and 99% of the full length oxynitrilase polypeptide.

“Improved enzyme properties” refers to an enzyme property that is better or more desirable for a specific purpose as compared to a reference oxynitrilase such as a wild-type oxynitrilase or another improved engineered oxynitrilase. Improved enzyme properties are exhibited by engineered oxynitrilase polypeptides in this disclosure. Enzyme properties that are expected to be improved include, but are not limited to, enzyme activity (which can be expressed as a percentage of substrate conversion), thermal stability, solvent stability, pH activity characteristics, cofactor requirements, tolerance to inhibitors (e.g., substrate or product inhibition), stereospecificity and stereoselectivity (including enantioselectivity and diastereoselectivity).

“Insertion” refers to the modification of a polypeptide by adding one or more amino acids from a reference polypeptide. In some embodiments, the improved engineered oxynitrilase comprises insertions of one or more amino acids to a naturally-occurring oxynitrilase polypeptide as well as insertions of one or more amino acids to other engineered oxynitrilase polypeptides. It can be inserted in the internal portions of the polypeptide or inserted to the carboxyl or amino terminus. As used herein, insertions include fusion proteins known in the art. The insertion can be a contiguous segment of amino acids or separated by one or more amino acids in naturally-occurring or engineered polypeptides.

An “isolated polypeptide” refers to a polypeptide that is substantially separated from other substances with which it is naturally associated, such as proteins, lipids, and polynucleotides. The term comprises polypeptides that have been removed or purified from their naturally occurring environment or expression system (e.g., in host cells or in vitro synthesis). Engineered oxynitrilase polypeptides may be present in the cell, in the cell culture medium, or prepared in various forms, such as lysates or isolated preparations. As such, in some embodiments, the engineered oxynitrilase polypeptide may be an isolated polypeptide.

“Naturally occurring” or “wild-type” refers to the form found in nature. For example, a naturally-occurring or wild-type polypeptide or polynucleotide sequence is a sequence that is present in an organism that can be isolated from sources in nature and which has not been intentionally modified by manual procedures.

“Oxynitrilase” or “HNL” as used herein, refers to a wild-type or engineered enzyme having oxynitrilase activity.

The terms “Polynucleotide” and “nucleic acid” are used interchangeably herein.

The terms “Protein”, “polypeptide” and “peptide” are used interchangeably herein to denote a polymer of at least two amino acids covalently linked by an amide bond, regardless of length or post-translational modification (e.g., glycosylation, phosphorylation, lipidation, myristoylation, ubiquitination, etc.). This definition includes D-amino acids and L-amino acids, as well as mixtures of D-amino acids and L-amino acids. Preferably, the amino acids have L configuration.

“Recombinant” or “engineered” or “non-naturally occurring” when used with reference to, for example, a cell, nucleic acid or polypeptide, refers to a material or material corresponding to the native or native form of the material, that has been modified in a manner that would not otherwise exist in nature, or is identical thereto but produced or derived from synthetic material and/or by manipulation using recombinant techniques.

“Reference sequence” refers to a defined sequence that is used as a basis for sequence comparison. The reference sequence may be a subset of a larger sequence, for example, a full-length gene or a fragment of a polypeptide sequence. In general, a reference sequence is at least nucleotides or amino acid residues in length, at least 25 residues long, at least 50 residues in length, or the full length of the nucleic acid or polypeptide. Because two polynucleotides or polypeptides may each (1) comprise a sequence (i.e., a portion of the complete sequence) that is similar between two sequences, and (2) may further comprise sequences that is divergent between the two sequences, sequence comparisons between two (or more) polynucleotides or polypeptides are typically performed by comparing the sequences of the two polynucleotides or polypeptides over a “comparison window” to identify and compare local regions of sequence similarity. In some embodiments, a “reference sequence” is not intended to be limited to a wild-type sequence, and may comprise engineered or altered sequences. For example, “a reference sequence with proline at the residue corresponding to X35 based on SEQ ID NO: 2” refers to a reference sequence wherein the corresponding residue at position X35 in SEQ ID NO: 2 which is alanine, has been altered to proline.

“Sequence identity” and “homology” are used interchangeably herein to refer to comparisons between polynucleotide sequences or polypeptide sequences (“sequence identity” and “homology” are generally expressed as a percentage), and are determined by comparing two optimally aligned sequences over a comparison window, where the portion of the polynucleotide or polypeptide sequence in the comparison window may comprise additions or deletions (i.e., gaps) as compared to the reference sequence for optimal alignment of the two sequences. The percentage can be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those skilled in the art will appreciate that there are many established algorithms available to align two sequences. The optimal alignment of sequences for comparison can be conducted, for example, by the local homology algorithm of Smith and Waterman, 1981, Adv. Appl. Math. 2: 482, by the Homology alignment algorithm of Needleman and Wunsch, 1970, J. Mol. Biol. 48: 443, by the search for similarity method of Pearson and Lipman, 1988, Proc. Natl. Acad. Sci. USA 85: 2444, by computerized implementations of these algorithms (GAP, BESTFIT, FASTA, and TFASTA in the GCG Wisconsin Package) or by visual inspection (see generally, Current Protocols in Molecular Biology, F M Ausubel et al. eds., Current Protocols, a Joint Venture between Greene Publishing Associates, Inc. and John Wiley &Sons, Inc., (1995 Supplement) (Ausubel)). Examples of algorithms that are suitable for determining the percent sequence identity and percent sequence similarity are the BLAST and BLAST 2.0 algorithms, which are described in Altschul et al., 1990, J. Mol. Biol. 215: 403-410 and Altschul et al., 1977, Nucleic Acids Res. 3389-3402, respectively. Software for performing BLAST analysis is publicly available through the National Center for Biotechnology Information website. The algorithm involves first identifying high scoring sequence pairs (HSPs) by identifying short words of length W in the query sequence, which either match or satisfy some positive-valued threshold scores T when aligned with a word of the same length in the database sequence. T is referred to as, the neighborhood word score threshold (Altschul et al., Supra). These initial neighborhood word hits serve as seeds for initiating searches to find longer HSPs that contain them. The word hits are then extended in both directions along each sequence for as far as the cumulative alignment score can be increased. For nucleotide sequences, the cumulative scores are calculated using the parameters M (reward score for matched pair of residues; always>0) and N (penalty score for mismatched residues; always <0). For amino acid sequences, a scoring matrix is used to calculate the cumulative score. The extension of the word hits in each direction are halted when: the cumulative alignment score falls off by the quality X from its maximum achieved value; the cumulative score goes 0 or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, the expected value (E) of 10, M=5, N=−4, and a comparison of both strands as a default value. For amino acid sequences, the BLASTP program uses as defaults the wordlength (W) of 3, the expected value (E) of 10 and the BLOSUM62 scoring matrix (see Henikoff and Henikoff, 1989, Proc Natl Acad Sci USA 89: 10915). Exemplary determination of sequence alignments and % sequence identity can employ the BESTFIT or GAP programs in the GCG Wisconsin Software package (Accelrys, Madison WI), using the default parameters provided.

“Solvent-stable” refers to an oxynitrilase polypeptide that maintains similar activity (for example more than 50% to 80%) after exposure to varying solvent (ethanol, isopropanol, dimethylsulfoxide, tetrahydrofuran, 2-Methyltetrahydrofuran, acetone, toluene, butyl acetate, methyl tert-butyl ether, etc.) for a period of time (e.g., 0.5-24 hours).

“Stereoisomers”, and similar expressions are used interchangeably herein to refer to all isomers resulting from a difference in orientation of atoms in their space only. It includes enantiomers and compounds that have more than one stereocenter and are not mirror images of one another (i.e., diastereomers).

“Stereoselectivity” refers to the preferential formation of one stereoisomer over the other in a chemical or enzymatic reaction. Stereoselectivity can be partial, with the formation of one stereoisomer is favored over the other; or it may be complete where only one stereoisomer is formed. When the stereoisomers are enantiomers, the stereoselectivity is referred to as enantioselectivity. It is often reported as “enantiomeric excess” (ee for short). When the stereoisomers are diastereomers, the stereoselectivity is referred to as diastereoselectivity. It is often reported as “diastereomeric excess” (de for short). The fraction, typically a percentage, is generally reported in the art as the enantiomeric excess (i.e., ee) derived therefrom according to the following formula: [major enantiomer−minor enantiomer]/[major enantiomer+minor enantiomer].

“Suitable reaction conditions” refer to those conditions (e.g., catalyst loading, substrate loading, temperature, solvent, etc.) in the reaction system, under which the substrate is converted to the desired product.

“Suitable reaction conditions” in the context of the biocatalytic processes of the present disclosure refer to those conditions (e.g., enzyme loading, substrate loading, cofactor loading, temperature, pH, buffer, co-solvent, etc.) in the biocatalytic reaction system, under which the oxynitrilase polypeptide of the present disclosure can convert a substrate to a desired product compound. Exemplary “suitable reaction conditions” are provided in the present disclosure and illustrated by examples.

“Thermostable” means that an oxynitrilase polypeptide that retains similar activity (e.g., greater than 50%) after being exposed to an elevated temperature (e.g., 30-85° C.) for a period of time (e.g., 0.5-24 h).

Furthermore, the use of a term designating a monovalent radical where a divalent radical is appropriate shall be construed to designate the respective divalent radical and vice versa. Unless otherwise specified, conventional definitions of terms control and conventional stable atom valences are presumed and achieved in all formulas and groups. The articles “a” and “an” refer to one or more than one (e.g., to at least one) of the grammatical object of the article. By way of example, “an element” means one element or more than one element.

The term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

In the groups, radicals, or moieties defined below, the number of carbon atoms is often specified preceding the group, for example, C₁-C₈alkyl means an alkyl group or radical having 1 to 8 carbon atoms. In general, for groups comprising two or more subgroups, the last named group is the radical attachment point, for example, “alkylaryl” means a monovalent radical of the formula alkyl-aryl-, while “arylalkyl” means a monovalent radical of the formula aryl-alkyl-. The term “halogen” or “halo” means fluorine, chlorine, bromine or iodine. The term “nitro” shall mean the radical —NO₂.

The term “alkyl” as used herein represents a saturated, branched or straight hydrocarbon group, e.g., having from 1 to 50 carbon atoms, e.g., C₁-C₃ alkyl, C₁-C₆ alkyl, C₂-C₈-alkyl, C₃-C₈-alkyl, C₁-C₈-alkyl, C₁-C₁₀ alkyl, C₁-C₂₀ alkyl, C₁-C₃₀ alkyl, C₁-C₄₀ alkyl, C₁-C₅₀ alkyl, and the like. Representative examples are methyl, ethyl, propyl (e.g., prop-1-yl, prop-2-yl (or iso-propyl)), butyl (e.g., 2-methylprop-2-yl (or tert-butyl), but-1-yl, but-2-yl), pentyl (e.g., pent-1-yl, pent-2-yl, pent-3-yl), 2-methylbut-1-yl, 3-methylbut-1-yl, hexyl (e.g., hex-1-yl), heptyl (e.g., hept-1-yl), octyl (e.g., oct-1-yl), nonyl (e.g., non-1-yl), and the like. The term “C₁-C₆ alkyl” is to be interpreted accordingly.

The term “alkenyl” as used herein represents a branched or straight hydrocarbon group having at least one double bond, e.g., having from respectively 2 to 50 carbon atoms and at least one double bond, e.g., C₂-C₃alkenyl, C₂-C₆ alkenyl, C₂-C₇ alkenyl, C₂-C₅ alkenyl, C₃-C₅ alkenyl, C₁-C₁₀-alkenyl, C₁-C₂₀ alkenyl, C₁-C₃₀ alkenyl, C₁-C₄₀ alkenyl, C₁-C₅₀ alkenyl, and the like.

Representative examples are ethenyl (or vinyl), propenyl (e.g., prop-1-enyl, prop-2-enyl), butadienyl (e.g., buta-1,3-dienyl), butenyl (e.g., but-1-en-1-yl, but-2-en-1-yl), pentenyl (e.g., pent-1-en-1-yl, pent-2-en-2-yl), hexenyl (e.g., hex-1-en-2-yl, hex-2-en-1-yl), 1-ethylprop-2-enyl, 1,1-(dimethyl)prop-2-enyl, 1-ethylbut-3-enyl, 1,1-(dimethyl)but-2-enyl, and the like.

The term “alkynyl” as used herein represents a branched or straight hydrocarbon group having at least one triple bond, e.g., having from respectively 2 to 50 carbon atoms and at least one triple bond, e.g., C₂-C₃ alkynyl, C₂-C₆ alkynyl, C₂-C₇ alkynyl, C₂-C₅ alkynyl, C₃-C₅ alkynyl, C₁-C₁₀ alkynyl, C₁-C₂₀ alkynyl, C₁-C₃₀ alkynyl, C₁-C₄₀ alkynyl, C₁-C₅₀ alkynyl, and the like. Representative examples are ethynyl, propynyl (e.g., prop-1-ynyl, prop-2-ynyl), butynyl (e.g., but-1-ynyl, but-2-ynyl), pentynyl (e.g., pent-1-ynyl, pent-2-ynyl), hexynyl (e.g., hex-1-ynyl, hex-2-ynyl), 1-ethylprop-2-ynyl, 1,1-(dimethyl)prop-2-ynyl, 1-ethylbut-3-ynyl, 1,1-(dimethyl)but-2-ynyl, and the like.

The term “aryl” as used herein is intended to include monocyclic, bicyclic or polycyclic carbocyclic aromatic rings. Representative examples are phenyl, naphthyl (e.g., naphth-1-yl, naphth-2-yl), anthryl (e.g., anthr-1-yl, anthr-9-yl), phenanthryl (e.g., phenanthr-1-yl, phenanthr-9-yl), and the like. Aryl is also intended to include monocyclic, bicyclic or polycyclic carbocyclic aromatic rings substituted with carbocyclic aromatic rings. Representative examples are biphenyl (e.g., biphenyl-2-yl, biphenyl-3-yl, biphenyl-4-yl), phenylnaphthyl (e.g., 1-phenylnaphth-2-yl, 2-phenylnaphth-1-yl), and the like. Aryl is also intended to include partially saturated bicyclic or polycyclic carbocyclic rings with at least one unsaturated moiety (e.g., a benzo moiety). Representative examples are, indanyl (e.g., indan-1-yl, indan-5-yl), indenyl) (e.g., inden-1-yl, inden-5-yl), 1,2,3,4-tetrahydronaphthyl (e.g., 1,2,3,4-tetrahydronaphth-1-yl, 1,2,3,4-tetrahydronaphth-2-yl, 1,2,3,4-tetrahydronaphth-6-yl), 1,2-dihydronaphthyl (e.g., 1,2-dihydronaphth-1-yl, 1,2-dihydronaphth-4-yl, 1,2-dihydronaphth-6-yl), fluorenyl (e.g., fluoren-1-yl, fluoren-4-yl, fluoren-9-yl), and the like. Aryl is also intended to include partially saturated bicyclic or polycyclic carbocyclic aromatic rings containing one or two bridges. Representative examples are, benzonorbornyl (e.g., benzonorborn-3-yl, benzonorborn-6-yl), 1,4-ethano-1,2,3,4-tetrahydronapthyl (e.g., 1,4-ethano-1,2,3,4-tetrahydronapth-2-yl, 1,4-ethano-1,2,3,4-tetrahydronapth-10-yl), and the like. Aryl is also intended to include partially saturated bicyclic or polycyclic carbocyclic aromatic rings containing one or more spiro atoms. Representative examples are spiro[cyclopentane-1,1′-indane]-4-yl, spiro[cyclopentane-1,1′-indene]-4-yl, spiro[piperidine-4,1′-indane]-1-yl, spiro[piperidine-3,2′-indane]-1-yl, spiro[piperidine-4,2′-indane]-1-yl, spiro[piperidine-4,1′-indane]-3′-yl, spiro[pyrrolidine-3,2′-indane]-1-yl, spiro[pyrrolidine-3,1′-(3′,4′-dihydronaphthalene)]-1-yl, spiro[piperidine-3,1′-(3′,4′-dihydronaphthalene)]-1-yl, spiro[piperidine-4,1′-(3′,4′-dihydronaphthalene)]-1-yl, spiro[imidazolidine-4,2′-indane]-1-yl, spiro[piperidine-4,1′-indene]-1-yl, and the like.

The term C₆-C₁₄ aryl is to be interpreted accordingly.

Preferably, aryl refers to a monocyclic or bicyclic carbocyclic aromatic ring.

The term “arylalkyl” (e.g., benzyl, phenylethyl, β-phenylpropyl, 1-naphtylmethyl, 2-(1-naphtyl)ethyl and the like) represents an aryl group as defined above attached through an alkyl chain having the indicated number of carbon atoms or substituted alkyl group as defined above. The term C₇-C₂₀ arylalkyl is to be construed accordingly.

Preferred examples of aryl include, but are not limited to, phenyl and naphthyl. In an embodiment, aryl is phenyl.

As used herein, the term “cycloalkyl” means a monocyclic or polycyclic saturated or partially unsaturated carbon ring, e.g., containing 3-20 carbon atoms e.g., containing 3-18 carbon atoms, wherein there are no delocalized pi electrons (aromaticity) shared among the ring carbon. The terms “C₃-C₂₀cycloalkyl”, and “C₃-C₁₀cycloalkyl” are to be construed accordingly. The term polycyclic encompasses bridged (e.g., norbonane), fused (e.g., decalin) and spirocyclic cycloalkyl. Preferably, cycloalkyl, e.g., “C₃-C₂₀cycloalkyl” and “C₃-C₁₅cycloalkyl”, is a monocyclic or spirocyclic hydrocarbon group of 3 to 20 and 3 to 18 carbon atoms, respectively.

Representative examples are spiro[2.5]octanyl, spiro[4.5]decanyl, cyclopropenyl, cyclopropyl, cyclobutyl, cyclobutenyl, cyclopentyl, cyclohexyl, cycloheptanyl, cyclooctanyl, spiro[2.3]hexanyl, spiro[3.3]heptyl, spiro[3.4]octanyl, spiro[3.5]nonanyl, spiro[4.5]decanyl, spiro[5.5]undecanyl, spiro[4.4]nonanyl, bicyclo[2.2.2]octanyl, bicyclo[2.2.2]octenyl, bicyclo[1.1.1]pentanyl, decahydronaphthalenyl, bicyclo[3.3.0]octanyl, adamantyl, norbornanyl, norbornenyl, nortricyclyl, bicycle-[3.2.1]octanyl, tricyclo[5.2.1.0^{2,6}]decanyl, bicyclo[2.2.1]heptyl, and the like.

As used herein, the term “fluoroalkyl” refers to straight chain or branched alkyl groups, as defined above, where some or all of the hydrogen atoms of these groups are replaced by fluorine atoms. The term C₁-C₆fluoroalkyl is to be construed accordingly. Examples include, without limitation, fluoromethyl, difluoromethyl, trifluoromethyl, 1-fluoroethyl, 2-fluoroethyl, 2,2-difluoroethyl, 2,2,2-trifluoroethyl, pentafluoroethyl, 3,3,3-trifluoroprop-1-yl, 1,1,1-trifluoroprop-2-yl, heptafluoroisopropyl, 1-fluorobzutyl, 2-fluorobutyl, 3-fluorobbutyl, 4-fluorobutyl, 4,4,4-trifluorobutyl, fluoro-tert-butyl, and the like.

As used herein, the term “haloalkyl” refers to an alkyl radical, as defined above, substituted by one or more halo radicals, as defined herein. The terms C₁-C₂₀haloalkyl and C₁-C₆haloalkyl are to be construed accordingly. Examples of haloalkyl include, but are not limited to, trifluoromethyl, difluoromethyl, fluoromethyl, trichloromethyl, 1,1-difluoroethyl, 2,2-difluoroethyl, 2,2,2-trifluoroethyl, 2-fluoropropyl, 1,1,1-trifluoropropyl, 2,2-difluoropropyl, 3,3-difluoropropyl and 1-fluoromethyl-2-fluoroethyl, 1,3-dibromopropan-2-yl, 3-bromo-2-fluoropropyl, 1,1,2,2-tetrafluoropropyl, and 1,4,4-trifluorobutan-2-yl.

As used herein, the term “heteroaryl” is intended to include monocyclic heterocyclic aromatic rings containing one or more heteroatoms selected from oxygen, nitrogen, and sulfur (0, N, and S). Representative examples are pyrrolyl, furanyl, thienyl, oxazolyl, thiazolyl, imidazolyl, pyrazolyl, isothiazolyl, isooxazolyl, triazolyl, (e.g., 1,2,4-triazolyl), oxadiazolyl, (e.g., 1,2,3-oxadiazolyl, 1,2,4-oxadiazolyl, 1,2,5-oxadiazolyl, 1,3,4-oxadiazolyl), thiadiazolyl (e.g., 1,2,3-thiadiazolyl, 1,2,4-thiadiazolyl, 1,2,5-thiadiazolyl, 1,3,4-thiadiazolyl), tetrazolyl, pyranyl, pyridinyl, pyridazinyl, pyrimidinyl, pyrazinyl, 1,2,3-triazinyl, 1,2,4-triazinyl, 1,3,5-triazinyl, thiadiazinyl, azepinyl, azecinyl, and the like.

Heteroaryl is also intended to include bicyclic heterocyclic aromatic rings containing one or more heteroatoms selected from oxygen, nitrogen, and sulfur (0, N, and S). Representative examples are indolyl, isoindolyl, benzofuranyl, benzothiophenyl, indazolyl, benzopyranyl, benzimidazolyl, benzothiazolyl, benzisothiazolyl, benzoxazolyl, benzisoxazolyl, benzoxazinyl, benzotriazolyl, naphthyridinyl, phthalazinyl, pteridinyl, purinyl, quinazolinyl, cinnolinyl, quinolinyl, isoquinolinyl, quinoxalinyl, oxazolopyridinyl, isooxazolopyridinyl, pyrrolopyridinyl, furopyridinyl, thienopyridinyl, imidazopyridinyl, imidazopyrimidinyl, pyrazolopyridinyl, pyrazolopyrimidinyl, pyrazolotriazinyl, thiazolopyridinyl, thiazolopyrimidinyl, imdazothiazolyl, triazolopyridinyl, triazolopyrimidinyl, and the like.

Heteroaryl is also intended to include polycyclic heterocyclic aromatic rings containing one or more heteroatoms selected from oxygen, nitrogen, and sulfur (0, N, and S). Representative examples are carbazolyl, phenoxazinyl, phenazinyl, acridinyl, phenothiazinyl, carbolinyl, phenanthrolinyl, and the like.

Heteroaryl is also intended to include partially saturated monocyclic, bicyclic or polycyclic heterocyclyls containing one or more heteroatoms selected oxygen, nitrogen, and sulfur (0, N, and S). Representative examples are imidazolinyl, indolinyl, dihydrobenzofuranyl, dihydrobenzothienyl, dihydrobenzopyranyl, dihydropyridooxazinyl, dihydrobenzodioxinyl (e.g., 2,3-dihydrobenzo[b][1,4]dioxinyl), benzodioxolyl (e.g., benzo[d][1,3]dioxole), dihydrobenzooxazinyl (e.g., 3,4-dihydro-2H-benzo[b][1,4]oxazine), tetrahydroindazolyl, tetrahydrobenzimidazolyl, tetrahydroimidazo[4,5-c]pyridyl, tetrahydroquinolinyl, tetrahydroisoquinolinyl, tetrahydroquinoxalinyl, and the like.

The heteroaryl ring structure may be substituted by one or more substituents. The substituents can themselves be optionally substituted. The heteroaryl ring may be bonded via a carbon atom or heteroatom.

The term “5-20 membered heteroaryl” is to be construed accordingly.

The term “monocyclic heteroaryl” as used herein is intended to include monocyclic heterocyclic aromatic rings as defined above.

The term “bicyclic heteroaryl” as used herein is intended to include bicyclic heterocyclic aromatic rings as defined above.

Examples of 5-20 membered heteroaryl include, but are not limited to, indolyl, imidazopyridyl, isoquinolinyl, benzooxazolonyl, pyridinyl, pyrimidinyl, pyridinonyl, benzotriazolyl, pyridazinyl, pyrazolotriazinyl, indazolyl, benzimidazolyl, quinolinyl, triazolyl, (e.g., 1,2,4-triazolyl), pyrazolyl, thiazolyl, oxazolyl, isooxazolyl, pyrrolyl, oxadiazolyl, (e.g., 1,2,3-oxadiazolyl, 1,2,4-oxadiazolyl, 1,2,5-oxadiazolyl, 1,3,4-oxadiazolyl), imidazolyl, pyrrolopyridinyl, tetrahydroindazolyl, quinoxalinyl, thiadiazolyl (e.g., 1,2,3-thiadiazolyl, 1,2,4-thiadiazolyl, 1,2,5-thiadiazolyl, 1,3,4-thiadiazolyl), pyrazinyl, oxazolopyridinyl, pyrazolopyrimidinyl, benzoxazolyl, indolinyl, isooxazolopyridinyl, dihydropyridooxazinyl, tetrazolyl, dihydrobenzodioxinyl (e.g., 2,3-dihydrobenzo[b][1,4]dioxinyl), benzodioxolyl (e.g., benzo[d][1,3]dioxole) and dihydrobenzooxazinyl (e.g., 3,4-dihydro-2H-benzo[b][1,4]oxazine).

The term “heterocyclyl” as used herein represents a saturated or partially saturated monocyclic or polycyclic ring containing one or more heteroatoms selected from nitrogen, oxygen, sulfur, S(═O) and S(═O)₂, and wherein there are no delocalized pi electrons (aromaticity) shared among the ring carbon or heteroatoms. The heterocyclyl ring structure may be substituted by one or more substituents. The substituents can themselves be optionally substituted. The heterocyclyl may be bonded via a carbon atom or heteroatom. The term polycyclic encompasses bridged, fused and spirocyclic heterocyclyl.

Representative examples are aziridinyl (e.g., aziridin-1-yl), azetidinyl (e.g., azetidin-1-yl, azetidin-3-yl), oxetanyl, pyrrolidinyl (e.g., pyrrolidin-1-yl, pyrrolidin-2-yl, pyrrolidin-3-yl), imidazolidinyl (e.g., imidazolidin-1-yl, imidazolidin-2-yl, imidazolidin-4-yl), oxazolidinyl (e.g., oxazolidin-2-yl, oxazolidin-3-yl, oxazolidin-4-yl), thiazolidinyl (e.g., thiazolidin-2-yl, thiazolidin-3-yl, thiazolidin-4-yl), isothiazolidinyl, piperidinyl (e.g., piperidin-1-yl, piperidin-2-yl, piperidin-3-yl, piperidin-4-yl), homopiperidinyl (e.g., homopiperidin-1-yl, homopiperidin-2-yl, homopiperidin-3-yl, homopiperidin-4-yl), piperazinyl (e.g., piperazin-1-yl, piperazin-2-yl), morpholinyl (e.g., morpholin-2-yl, morpholin-3-yl, morpholin-4-yl), thiomorpholinyl (e.g., thiomorpholin-2-yl, thiomorpholin-3-yl, thiomorpholin-4-yl), 1-oxothiomorpholinyl, 1,1-dioxo-thiomorpholinyl, tetrahydrofuranyl (e.g., tetrahydrofuran-2-yl, tetrahydrofuran-3-yl), tetrahydrothienyl, tetrahydro-1,1-dioxothienyl, tetrahydropyranyl (e.g., 2-tetrahydropyranyl), tetrahydrothiopyranyl (e.g., 2-tetrahydrothiopyranyl), 1,4-dioxanyl, 1,3-dioxanyl, and the like. Heterocyclyl is also intended to represent a saturated 6 to 8 membered bicyclic ring containing one or more heteroatoms selected from nitrogen, oxygen, sulfur, S(═O) and S(═O)₂. Representative examples are octahydroindolyl (e.g., octahydroindol-1-yl, octahydroindol-2-yl, octahydroindol-3-yl, octahydroindol-5-yl), decahydroquinolinyl (e.g., decahydroquinolin-1-yl, decahydroquinolin-2-yl, decahydroquinolin-3-yl, decahydroquinolin-4-yl, decahydroquinolin-6-yl), decahydroquinoxalinyl (e.g., decahydroquinoxalin-1-yl, decahydroquinoxalin-2-yl, decahydroquinoxalin-6-yl) and the like. Heterocyclyl is also intended to represent a saturated 6 to 8 membered ring containing one or more heteroatoms selected from nitrogen, oxygen, sulfur, S(═O) and S(═O)₂ and having one or two bridges. Representative examples are 3-azabicyclo[3.2.2]nonyl, 2-azabicyclo[2.2.1]heptyl, 3-azabicyclo[3.1.0]hexyl, 2,5-diazabicyclo[2.2.1]heptyl, atropinyl, tropinyl, quinuclidinyl, 1,4-diazabicyclo[2.2.2]octanyl, and the like. Heterocyclyl is also intended to represent a 6 to 8 membered saturated ring containing one or more heteroatoms selected from nitrogen, oxygen, sulfur, S(═O) and S(═O)₂ and containing one or more spiro atoms. Representative examples are 1,4-dioxaspiro[4.5]decanyl (e.g., 1,4-dioxaspiro[4.5]decan-2-yl, 1,4-dioxaspiro[4.5]decan-7-yl), 1,4-dioxa-8-azaspiro[4.5]decanyl (e.g., 1,4-dioxa-8-azaspiro[4.5]decan-2-yl, 1,4-dioxa-8-azaspiro[4.5]decan-8-yl), 8-azaspiro[4.5]decanyl (e.g., 8-azaspiro[4.5]decan-1-yl, 8-azaspiro[4.5]decan-8-yl), 2-azaspiro[5.5]undecanyl (e.g., 2-azaspiro[5.5]undecan-2-yl), 2,8-diazaspiro[4.5]decanyl (e.g., 2,8-diazaspiro[4.5]decan-2-yl, 2,8-diazaspiro[4.5]decan-8-yl), 2,8-diazaspiro[5.5]undecanyl (e.g., 2,8-diazaspiro[5.5]undecan-2-yl), 1,3,8-triazaspiro[4.5]decanyl (e.g., 1,3,8-triazaspiro[4.5]decan-1-yl, 1,3,8-triazaspiro[4.5]decan-3-yl, 1,3,8-triazaspiro[4.5]decan-8-yl), and the like.

The term “β- to 14-membered heterocyclyl” is to be construed accordingly.

As used herein, the term “optional” or “optionally substituted” means that the described event or circumstance may or may not occur; for example, “optionally substituted aryl” refers to an aryl group that may or may not be substituted. This description includes both substituted aryl groups and unsubstituted aryl groups.

The term “pharmaceutically acceptable” is defined as being suitable for administration to humans without adverse events.

As used herein, the terms “sponge Nickel and sponge Cobalt” are intended to refer to nickel and cobalt catalysts having a skeletal structure formed from a nickel or cobalt alloy. Typically, these catalysts are formed from nickel or cobalt alloyed with aluminum and the aluminum subsequently removed. Included with the terms sponge nickel and sponge cobalt are the trademarked and well known Raney nickel and Raney cobalt catalysts.

Certain of the defined terms may occur more than once in the structural formulae, and upon such occurrence each term shall be defined independently of the other.

### Oxynitilases of the Disclosure

In another aspect, the oxynitrilase polypeptides disclosed herein can asymmetrically couple aldehyde or ketone substrates and nitroalkane substrates.

In some embodiments, engineered oxynitrilase polypeptides of the present disclosure are capable of converting 1,1-trifluoropropan-2-one and nitromethane to produce (S)-1,1,1-trifluoro-2-methyl-3-nitropropan-2-ol (i.e., (IA)) at a stereoselectivity at least equal to or greater than that of SEQ ID No: 606 and/or SEQ ID No: 2. The engineered oxynitrilase polypeptides of the present disclosure are capable of producing the β-nitro alcohol product, e.g., compound IA, in an enantiomeric excess of at least 60%, 65%, 70%, 75%, 80%, 85%, 90%, 91%, 92%, 93%, 94%, 95%, 96%, 97%, 98% or 99%.

In some embodiments, the engineered oxynitrilase polypeptides are capable of converting 1,1-trifluoropropan-2-one and nitromethane to produce (S)-1,1,1-trifluoro-2-methyl-3-nitropropan-2-ol (i.e., (IA)) at a stereoselectivity higher than that of the polypeptide of SEQ ID NO: 606 under suitable reaction conditions, e.g., those disclosed herein. The engineered oxynitrilase polypeptides comprise an amino acid sequence that is at least 80%, 85%, 86%, 87%, 88%, 89%, 90%, 91%, 92%, 93%, 94%, 95%, 96%, 97%, 98%, 99% or more identical to the sequence of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 608, 610, 612, 614, 616, 618, 620, 622, 624, 626, 628, 630, 632, 634, 636, 638, and 640. The identity between two amino acid sequences or two nucleotide sequences can be obtained by commonly used algorithms in the art and can be calculated according to default parameters by using NCBI Blastp and Blastn software, or by using the Clustal W algorithm (Nucleic Acid Research, 22 (22): 4673-4680, 1994). For example, using the Clustal W algorithm, the amino acid sequence identity of SEQ ID NO: 4 to SEQ ID NO: 606 is 92.78%.

The engineered oxynitrilase polypeptides represented by SEQ ID NO: 4 to 604, and 608 to 640 exhibit higher activity and/or stereoselectivity than that of SEQ ID NO: 606, as shown in the Examples.

In some embodiments, engineered oxynitrilase polypeptides comprise an amino acid sequence with insertions of one or more than one amino acid residues in SEQ ID NO: 2 and having oxynitrilase activity. For each and every embodiment of the engineered oxynitrilase polypeptides of the present disclosure, the insertion fragment may comprise 1 or more amino acids, 2 or more amino acids, 3 or more amino acids, 4 or more amino acids, 5 or more amino acids, 6 or more amino acids, 8 or more amino acids, 10 or more amino acids, 15 or more amino acids, or 20 or more amino acids, where the relevant functional and/or improved properties of the engineered oxynitrilase described herein are maintained. The insertion fragment can be inserted at the amino terminus or carboxy terminus, or the internal portion of the oxynitrilase polypeptide. In some embodiments, the insertion fragments may comprise 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, 1-50, 1-55, or 1-60 amino acid residues. In some embodiments, the number of insertion occurrence can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60 or more. In some embodiments, the insertion fragments may comprise 1, 2, 3, 4, 5, 6, 7, 8, 9, 10, 11, 12, 13, 14, 15, 16, 17, 18, 19, 20, 21, 22, 23, 24, 25 or more amino acid residues.

In some embodiments, the engineered oxynitrilase polypeptides comprise an amino acid sequence that differs from the sequence of SEQ ID NO: 2 in one or more residue positions selected from: X2, X12, X28, X29, X32, X39, X50, X55, X64, X105, X111, X147, X152, X154, X160, X185, X196, X203, X208, X209, X232, X233, and X250. In some embodiments, the engineered oxynitrilase polypeptides comprise an amino acid sequence comprising at least one of the following features (these features are substitutions of amino acid residues to the reference sequence of SEQ ID NO: 2: VX12I; SX28G; AX29W; NX32T; WX39F; WX39V; QX50E; QX50D; RX55G; LX64A; AX105G; DX111S; EX147K; TX152L; NX160M; TX185R; SX196G; YX203C; QX208R; QX208S; IX209V; SX232G; AX233G; or QX250G.

In a particular embodiment, the engineered oxynitrilase polypeptides comprise an amino acid sequence that differs from the sequence of SEQ ID NO: 2 in one or more residue positions selected from: X39, X105, and X154. In a particular embodiment, the engineered oxynitrilase polypeptides comprise an amino acid sequence comprising at least one of the following features (these features are substitutions of amino acid residues to the reference sequence of SEQ ID NO: 2): WX39F; WX39V; or AX105G.

In some embodiments, the engineered oxynitrilase polypeptides comprise an amino acid sequence that differs from the sequence of SEQ ID NO: 606 in one or more residue positions selected from: X2, X11, X12, X28, X29, X32, X33, X39, X43, X44, X46, X50, X55, X64, X80, X103, X105, X111, X118, X121, X147, X152, X154, X160, X172, X180, X185, X196, X203, X208, X209, X232, X233, X238, X241, X250, and X263. In some embodiments, the engineered oxynitrilase polypeptides comprise an amino acid sequence comprising at least one of the following features (these features are substitutions of amino acid residues to the reference sequence of SEQ ID NO: 606): TX11S; IX12V; SX28G; AX29W; NX32T; AX33V; VX39F, IX43S; DX44N; RX46H; QX50E; QX50D; EX55R; EX55G; LX64A; SX80A; HX103V; AX105G; DX111S; YX118V; FX121Y; EX147K; TX152L; NX160M; LX172R; EX180L; TX185R; SX196G; YX203C; QX208R; QX208S; IX209V; SX232G; AX233G; QX238M; KX241R; QX250G; or AX263S.

In a particular embodiment, the engineered oxynitrilase polypeptides comprise an amino acid sequence that differs from the sequence of SEQ ID NO: 606 in one or more residue positions selected from: X2, X105, X111, X154, X160, X185, X209, X232, and X250. In a particular embodiment, the engineered oxynitrilase polypeptides comprise an amino acid sequence comprising at least one of the following features (these features are substitutions of amino acid residues to the reference sequence of SEQ ID NO: 606): AX105G; DX111S; NX160M; TX185R; IX209V; SX232G; or QX250G.

In another aspect, this disclosure provides polynucleotides comprising sequences encoding engineered oxynitrilase polypeptides, expression vectors and host cells capable of expressing engineered oxynitrilase polypeptides. In some embodiments, the host cell can be a bacterial host cell, such as E. coli. The host cell can be used to express and isolate the engineered oxynitrilase described herein, or alternatively be directly used in the reaction for conversion of substrates to products.

In some embodiments, the engineered oxynitrilase in the form of whole cell, crude extract, isolated enzyme, or purified enzyme can be used alone or in an immobilized form, such as immobilization on a resin.

Polynucleotides, Control Sequences, Expression Vectors and Host Cells that can be Used to Produce Engineered Oxynitrilase Polypeptides

In another aspect, this disclosure provides polynucleotides encoding engineered polypeptides having oxynitrilase activity described herein. The polynucleotides can be linked to one or more heterologous regulatory sequences that control gene expression to produce recombinant polynucleotides that are capable of expressing the engineered polypeptides. Expression constructs comprising a heterologous polynucleotide encoding an engineered oxynitrilase may be introduced into a suitable host cell to express the corresponding engineered oxynitrilase polypeptide.

As apparent to one skilled in the art, the availability of protein sequences and knowledge of codons corresponding to a variety of amino acids provide an illustration of all possible polynucleotides that encode the protein sequence of interest. The degeneracy of the genetic code, in which the same amino acid is encoded by selectable or synonymous codons, allows for the production of an extremely large number of polynucleotides, all of which encode the engineered oxynitrilase polypeptides disclosed herein. Thus, upon determination of a particular amino acid sequence, one skilled in the art can generate any number of different polynucleotides by modifying one or more codons in a manner that does not alter the amino acid sequence of the protein. In this regard, this disclosure specifically contemplates each and every possible alteration of a polynucleotide that can be made by selecting a combination based on possible codon selections, for any of the polypeptides disclosed herein, comprising those amino acid sequences of exemplary engineered polypeptides listed in Examples 6 to 12 and 19, and any of the polypeptides disclosed as even sequence identifiers of SEQ ID NOS: 4 to 604, and 608 to 640 in the Sequence Listing incorporated by reference.

In various embodiments, the codons are preferably selected to accommodate the host cell in which the recombinant protein is produced. For example, codons preferred for bacteria are used to express genes in bacteria; codons preferred for yeast are used to express genes in yeast; and codons preferred for mammals are used for gene expression in mammalian cells.

In some embodiments, the polynucleotides encode polypeptides comprising amino acid sequences that are at least about 80%, 85%, 86%, 87%, 88%, 89%, 90%, 91%, 92%, 93%, 94%, 95%, 96%, 97%, 98%, 99%, or more identical to a reference sequence that is an even sequence identifier of SEQ ID NO: 4 to 604, and 608 to 640. Wherein the polypeptides have oxynitrilase activity and one or more of the improved properties described herein, for example, the ability to convert 1,1,1-trifluoropropan-2-one to compound (IA) with increased stereoselectivity compared to the polypeptide of SEQ ID NO: 2 and/or 606.

In some embodiments, the polynucleotides encode engineered oxynitrilase polypeptides comprising amino acid sequences having a percentage of identity described above and having one or more amino acid residue differences as compared to SEQ ID NO: 606.

In some embodiments, the present disclosure provides engineered polypeptides having oxynitrilase activity, wherein the engineered polypeptide has at least 80% sequence identity to the reference sequence of SEQ ID NO: 2 and comprises a combination of residue difference that is selected from the following positions: X2, X12, X28, X29, X32, X39, X50, X55, X64, X105, X111, X147, X152, X154, X160, X185, X196, X203, X208, X209, X232, X233, X250.

In some embodiments, the polynucleotides encoding the engineered oxynitrilase polypeptides comprise sequences having odd sequence identifier of SEQ ID NO: 3 to 603, and 607 to 639.

In some embodiments, the polynucleotides encode polypeptides as described herein; but at the nucleotide level, the polynucleotides have about 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more sequence identity to reference polynucleotides encoding engineered oxynitrilase polypeptides as described herein. In some embodiments, the reference polynucleotides are selected from the sequences having the odd sequence identifiers of SEQ ID NOs: 3 to 603, and 607 to 639.

The isolated polynucleotides encoding engineered oxynitrilase polypeptides can be manipulated to enable the expression of the engineered polypeptides in a variety of ways, which comprises further modification of the sequences by codon optimization to improve expression, insertion into suitable expression elements with or without additional control sequences, and transformation into a host cell suitable for expression and production of the engineered polypeptides.

Depending on the expression vector, manipulation of the isolated polynucleotide prior to insertion of the isolated polynucleotide into the vector may be desirable or necessary. Techniques for modifying polynucleotides and nucleic acid sequences using recombinant DNA methods are well known in the art. Guidance is provided below: Sambrook et al., 2001, Molecular Cloning: A Laboratory Manual, Third Edition, Cold Spring Harbor Laboratory Press; and Current Protocols in Molecular Biology, Ausubel. F. Eds., Greene Pub. Associates, 1998, 2010 Year update.

In another aspect, this disclosure also relates to recombinant expression vectors, depending on the type of host they are to be introduced into, including a polynucleotide encoding an engineered oxynitrilase polypeptide or variant thereof, and one or more expression regulatory regions, such as promoters and terminators, origin of replication and the like. Alternatively, the nucleic acid sequence of the present disclosure can be expressed by inserting the nucleic acid sequence or the nucleic acid construct comprising the sequence into an appropriate expression vector. In generating the expression vector, the coding sequence is located in the vector such that the coding sequence is linked to a suitable control sequence for expression. The recombinant expression vector can be any vector (e.g., a plasmid or virus) that can be conveniently used in recombinant DNA procedures and can result in the expression of a polynucleotide sequence. The choice of vector will generally depend on the compatibility of the vector with the host cell to be introduced into. The vector can be linear or closed circular plasmid. The expression vector may be an autonomously replicating vector, i.e., a vector that exists as an extrachromosomal entity whose replication is independent of chromosomal replication such as plasmids, extrachromosomal elements, minichromosomes, or artificial chromosomes. The vector may contain any tools for ensuring self-copying. Alternatively, the vector may be a vector that, when introduced into a host cell, integrates into the genome and replicates with the chromosome into which it is integrated. Moreover, a single vector or plasmid or two or more vectors or plasmids that together comprise the total DNA to be introduced into the genome of the host cell may be used.

Many expression vectors useful to the embodiments of the present disclosure are commercially available. An exemplary expression vector can be prepared by inserting a polynucleotide encoding an engineered oxynitrilase polypeptide to plasmid pACYC-Duet-1 (Novagen), pBR322 Vector (New England Biolabs), pUC19 Vector(New England Biolabs) or pET T7 Expression Vectors (Novagen).

In another aspect, this disclosure provides host cells comprising polynucleotides encoding engineered oxynitrilase polypeptides of the present disclosure. The polynucleotide is linked to one or more control sequences for expression of oxynitrilase polypeptides in a host cell. Host cells for expression of polypeptides encoded by the expression vectors of the present disclosure are well known in the art, including, but not limited to, bacterial cells such as E. coli, Streptomyces, and Salmonella typhimurium; fungals (e.g., Saccharomyces cerevisiae or Pichia pastoris); insect cells such as Drosophila S2 and Spodoptera Sf9; animal cells such as CHO, COS, BHK, 293 and Bowes melanoma cells; and plant cells. An exemplary host cell is E. coli BL21 (DE3). The above host cells may be wild-type or may be engineered cells through genomic edition, such as knockout of the wild-type oxynitrilase gene carried in the host cell's genome. Suitable media and growth conditions for the above host cells are well known in the art.

Polynucleotides used to express engineered oxynitrilases can be introduced into cells by a variety of methods known in the art. Techniques comprise, among others, electroporation, bio-particle bombardment, liposome-mediated transfection, calcium chloride transfection, and protoplast fusion. Different methods of introducing polynucleotides into cells are known to those skilled in the art.

### Process of Producing an Engineered Oxynitrilase Polypeptide

Engineered oxynitrilase can be obtained by subjecting a polynucleotide encoding an oxynitrilase to mutagenesis and/or directed evolution. An exemplary directional evolution technique can be found in “Biocatalysis for the Pharmaceutical Industry: Discovery, Development, and Manufacturing” (2009 John Wiley &Sons Asia (Pte) Ltd. ISBN: 978-0-470-82314-9).

When the sequence of an engineered polypeptide is known, the encoding polynucleotide may be prepared by standard solid-phase methods according to known synthetic methods. In some embodiments, fragments of up to about 100 bases can be synthesized separately and then ligated (e.g., by enzymatic or chemical ligation methods or polymerase-mediated methods) to form any desired contiguous sequence. For example, the polynucleotides and oligonucleotides of the present disclosure can be prepared by chemical synthesis using, for example, the classic phosphoramidite methods described by Beaucage et al., 1981, Tet Lett 22: 1859-69, or Matthes et al. People, 1984, EMBO J. 3: 801-05, as typically practiced in automated synthesis methods. According to the phosphoramidite method, oligonucleotides are synthesized, purified, annealed, ligated, and cloned into a suitable vector, for example, in an automated DNA synthesizer. In addition, essentially any nucleic acid is available from any of a variety of commercial sources.

In some embodiments, the present disclosure also provides a process for preparing or producing an engineered oxynitrilase polypeptide that is capable of converting 1,1,1-trifluoropropan-2-one to Compound (IA) under suitable reaction conditions, wherein the process comprises culturing a host cell capable of expressing a polynucleotide encoding an engineered polypeptide under culture conditions suitable for the expression of the polypeptide. In some embodiments, the process of preparing a polypeptide further comprises isolating the polypeptide. Engineered polypeptides may be expressed in suitable cells and isolated (or recovered) from the host cell and/or culture medium using any one or more of the well-known techniques for protein purification, the techniques for protein purification include, among others, lysozyme treatment, sonication, filtration, salting out, ultracentrifugation and chromatography.

### Methods of Using an Engineered Oxynitrilase and Compounds Prepared Therewith

The present disclosure also provides a process of preparing a wide range of compounds or structural analogs thereof using an engineered oxynitrilase polypeptide disclosed herein.

Thus, the present disclosure provides a process for the asymmetric synthesis of a β-nitro alcohol using an engineered oxynitrilase polypeptide disclosed herein, the process comprising the step of contacting a nitroalkane and an aldehyde or ketone substrate with the oxynitrilase polypeptide disclosed herein, to obtain a β-nitro alcohol product.

The choice of applicable electrophiles ranges from aromatic to heteroaromatic and aliphatic aldehydes and ketones. Depending on the substrate and reaction systems yields up to at least 90% or enantiomeric excess >99% could be obtained by the process of the disclosure.

In an embodiment, the aldehyde or ketone substrate comprises an electron withdrawing substituent.

In an embodiment, the present disclosure also provides a process for the asymmetric synthesis of a β-nitro alcohol using the herein disclosed engineered oxynitrilase polypeptides, the resulting β-nitro alcohol having the structure shown in formula (I):

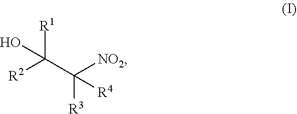

wherein:
R¹ and R² are each independently selected from H, alkyl, e.g., C₁-C₂₀alkyl, alkenyl, e.g., C₂-C₂₀alkenyl, alkynyl, e.g., C₂-C₂₀alkynyl, cycloalkyl, e.g., C₃-C₁₀cycloalkyl, aryl, e.g., C₆-C₁₄aryl, arylalkyl, e.g., C₇-C₂₀arylalkyl, heterocyclyl, e.g., 3-14 membered heterocyclyl, and heteroaryl, e.g., 5-20 membered heteroaryl,
wherein the alkyl, alkenyl, and alkynyl are each optionally substituted by one or more R^{a} e.g., one to six R^{a},
wherein the cycloalkyl, aryl, arylalkyl, heterocyclyl, and heteroaryl are each optionally substituted by one or more R^{b}, e.g., one to six R^{b};
R³ and R⁴ are each independently selected from H and alkyl, e.g., C₁-C₂₀alkyl, wherein the alkyl, e.g., C₁-C₂₀alkyl, is optionally substituted by one or more R^{b}, e.g., one to six R^{b};
each R^{a} is at each occurrence independently selected from cycloalkyl, e.g., C₃-C₁₀cycloalkyl, aryl, e.g., C₆-C₁₄aryl, arylalkyl, e.g., C₇-C₂₀arylalkyl, heterocyclyl, e.g., 3-14 membered heterocyclyl, and heteroaryl, e.g., 5-20 membered heteroaryl, halogen, e.g., F, haloalkyl, e.g., C₁-C₂₀haloalkyl, e.g., —CF₃, —CN, —OR^{c}, —NR^{c}R^{c}, —(CH₂)ₙCOOR^{c}, —(CH₂)ₙ—C(═O)R^{c}, —(CH₂)ₙ—C(═O)NR^{c}R^{c};
each R^{b} is at each occurrence independently selected from halogen, e.g., F, haloalkyl, e.g., C₁-C₂₀haloalkyl, e.g., —CF₃, —CN, —NO₂, —OR^{c}, —NR^{c}R^{c}, —(CH₂)ₙCOOR^{c}, —(CH₂)ₙ—C(═O)R^{c}, —(CH₂)ₙ—C(═O)NR^{c}R^{c}, C₁-C₂₀alkyl, C₂-C₂₀alkenyl, or C₂-C₂₀alkynyl;
each R^{c} is at each occurrence independently selected from H, C₁-C₂₀alkyl, C₂-C₂₀alkenyl, and C₂-C₂₀alkynyl, each optionally substituted by one or more R^{b}, e.g., one to six R^{b}; and
n is 0, 1, 2, 3, 4, 5 or 6, e.g., 0, 1, 2 or 3;
the process comprising the step of contacting a nitroalkane of formula (II), which is R³R⁴CHNO₂, and an aldehyde or ketone substrate, with an oxynitrilase polypeptide as disclosed herein, to obtain a β-nitro alcohol product of formula (I), the aldehyde or ketone substrate having the formula (III),

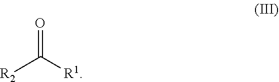

In an embodiment, the engineered oxynitrilase polypeptides can be used in a process of preparing a β-nitro alcohol compound, e.g., a compound of structural formula (I) as defined above.

The addition of a nitroalkane other than nitromethane to the aldehyde or ketone substrate produces two new stereocenters simultaneously. The addition of nitromethane to the aldehyde or ketone substrate produces one new stereocenter.

Thus, in an embodiment, the β-nitro alcohol product, e.g., a compound of structural formula (I) is in diastereomeric excess over the other diastereomers. In an embodiment, the (β-nitro alcohol product is present in an diastereomeric excess of at least 60%, 70%, 80%, 85%, 90%, 91%, 92%, 93%, 94%, 95%, 96%, 97%, 98%, 99%, or more.

In an embodiment, the β-nitro alcohol product, e.g., a compound of structural formula (I) is in enantiomeric excess over the other enantiomers. In an embodiment, the β-nitro alcohol product is present in an enantiomeric excess of at least 60%, 70%, 80%, 85%, 90%, 91%, 92%, 93%, 94%, 95%, 96%, 97%, 98%, 99%, or more.

In an embodiment, the oxynitrilase polypeptides have at least 80%, 85%, 86%, 87%, 88%, 89%, 90%, 91%, 92%, 93%, 94%, 95%, 96%, 97%, 98%, 99% or more of sequence identity with SEQ ID NO: 2, and are capable of coupling the aldehyde or ketone substrate, e.g., of formula (III) and the nitroalkane substrate, e.g., of formula (II), e.g., nitromethane, to form a β-nitro alcohol product, e.g., of formula (I) with a higher conversion and/or higher stereoselectivity than SEQ ID NO: 606.

As disclosed herein, the oxynitrilase polypeptides useful in the process of the present disclosure may be characterized according to the ability to condense 1,1,1-trifluoropropan-2-one and nitromethane to form (S)-1,1,1-trifluoro-2-methyl-3-nitropropan-2-ol (IA).

Thus, in any of the embodiments of the process disclosed herein, the process may be carried out, wherein the oxynitrilase polypeptides are capable of coupling 1,1,1-trifluoropropan-2-one and nitromethane to form (S)-1,1,1-trifluoro-2-methyl-3-nitropropan-2-ol (IA) with a higher conversion and/or higher stereoselectivity than SEQ ID NO: 606, and have at least 80%, 85%, 86%, 87%, 88%, 89%, 90%, 91%, 92%, 93%, 94%, 95%, 96%, 97%, 98%, 99% or more of sequence identity with SEQ ID NO: 2.

In an embodiment, the resulting β-nitro alcohol has the structure shown in formula (I-i):

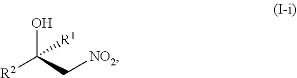

wherein:
R¹ and R² are each independently selected from H, alkyl, e.g., C₁-C₂₀alkyl, alkenyl, e.g., C₂-C₂₀alkenyl, alkynyl, e.g., C₂-C₂₀alkynyl, cycloalkyl, e.g., C₃-C₁₀cycloalkyl, aryl, e.g., C₆-C₁₄aryl, arylalkyl, e.g., C₇-C₂₀arylalkyl, heterocycloalkyl, e.g., 3-14 membered heterocycloalkyl, and heteroaryl, e.g., 5-20 membered heteroaryl,
wherein the alkyl, alkenyl, and alkynyl are each optionally substituted by one or more R^{a} e.g., one to six R^{a},
wherein the cycloalkyl, aryl, arylalkyl, heterocycloalkyl, and heteroaryl are each optionally substituted by one or more R^{b}, e.g., one to six R^{b};
each R^{a} is at each occurrence independently selected from cycloalkyl, e.g., C₃-C₁₀cycloalkyl, aryl, e.g., C₆-C₁₄aryl, arylalkyl, e.g., C₇-C₂₀arylalkyl, heterocycloalkyl, e.g., 3-14 membered heterocycloalkyl, and heteroaryl, e.g., 5-20 membered heteroaryl, halogen, e.g., F, haloalkyl, e.g., C₁-C₂₀haloalkyl, e.g., —CF₃, —CN, —OR^{c}, —NR^{c}R^{c}, —(CH₂)ₙCOOR^{c}, —(CH₂)ₙ—C(═O)R^{c}, —(CH₂)ₙ—C(═O)NR^{c}R^{c};
each R^{b} is at each occurrence independently selected from halogen, e.g., F, haloalkyl, e.g., C₁-C₂₀haloalkyl, e.g., —CF₃, —CN, —NO₂, —OR^{c}, —NR^{c}R^{c}, —(CH₂)ₙCOOR^{c}, —(CH₂)ₙ—C(═O)R^{c}, —(CH₂)ₙ—C(═O)NR^{c}R^{c}, C₁-C₂₀alkyl, C₂-C₂₀alkenyl, or C₂-C₂₀alkynyl;
each R^{c} is at each occurrence independently selected from H, C₁-C₂₀alkyl, C₂-C₂₀alkenyl, and C₂-C₂₀alkynyl, each optionally substituted by one or more R^{b}, e.g., one to six R^{b}; and
n is 0, 1, 2, 3, 4, 5 or 6, e.g., 0, 1, 2 or 3;
the process comprising the step of contacting nitromethane and an aldehyde or ketone substrate with the oxynitrilase polypeptide according to the disclosure, to obtain a j-nitro alcohol product of formula (I-i), the aldehyde or ketone substrate having the formula (III),

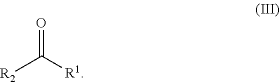

In any of the embodiments of the process disclosed herein, R¹ and R² are each independently selected from H, C₁-C₂₀alkyl, C₂-C₂₀alkenyl, C₂-C₂₀alkynyl, C₃-C₁₀cycloalkyl, C₆-C₁₄aryl, C₇-C₂₀arylalkyl, 3-14 membered heterocycloalkyl, and 5-20 membered heteroaryl,
wherein the C₁-C₂₀alkyl, C₂-C₂₀alkenyl, and C₂-C₂₀alkynyl are each optionally substituted by one to six R^{a},
wherein the C₃-C₁₀cycloalkyl, C₆-C₁₄aryl, C₇-C₂₀arylalkyl, 3-14 membered heterocycloalkyl, and 5-20 membered heteroaryl are each optionally substituted by one to six R^{b};
each R^{a} is at each occurrence independently selected from C₃-C₁₀cycloalkyl, C₆-C₁₄aryl, 3-14 membered heterocycloalkyl, 5-20 membered heteroaryl, halogen, e.g., F, haloalkyl, e.g., C₁-C₂₀haloalkyl, e.g., —CF₃, —CN, —OR, —NR^{c}R^{c}, —(CH₂)ₙCOOR^{c}, —(CH₂)ₙ—C(═O)R^{c}, —(CH₂)ₙ—C(═O)NR^{c}R^{c};
each R^{b} is at each occurrence independently selected from halogen, e.g., F, haloalkyl, e.g., C₁-C₂₀haloalkyl, e.g., —CF₃, —CN, —NO₂, —OR^{c}, —NR^{c}R^{c}, —(CH₂)ₙCOOR^{c}, —(CH₂)ₙ—C(═O)R^{c}, —(CH₂)ₙ—C(═O)NR^{c}R^{c}, C₁-C₂₀alkyl, C₂-C₂₀alkenyl, or C₂-C₂₀alkynyl;
each R^{c} is at each occurrence independently selected from H, C₁-C₂₀alkyl, C₂-C₂₀alkenyl, and C₂-C₂₀alkynyl; and
n is 0, 1, 2, 3, 4, 5 or 6, e.g., 0, 1, 2 or 3.

In any of the embodiments of the process disclosed herein, R¹ and R² are each independently selected from H, C₁-C₂₀alkyl, C₂-C₂₀alkenyl, C₂-C₂₀alkynyl, C₃-C₁₀cycloalkyl, C₆-C₁₄aryl, C₇-C₂₀arylalkyl, 3-14 membered heterocycloalkyl, and 5-20 membered heteroaryl,
wherein the C₁-C₂₀alkyl, C₂-C₂₀alkenyl, and C₂-C₂₀alkynyl are each optionally substituted by one to six R^{a},
wherein the C₃-C₁₀cycloalkyl, C₆-C₁₄aryl, C₇-C₂₀arylalkyl, 3-14 membered heterocycloalkyl, and 5-20 membered heteroaryl are each optionally substituted by one to six R^{b}.
each R^{a} is at each occurrence independently selected from C₃-C₁₀cycloalkyl, C₆-C₁₄aryl, 3-14 membered heterocyclyl, 5-20 membered heteroaryl, halogen, e.g., F, haloalkyl, e.g., C₁-C₂₀haloalkyl, e.g., —CF₃, —CN, —OR^{c}, and —NR^{c}R^{c};
each R^{b} is at each occurrence independently selected from halogen, e.g., F, haloalkyl, e.g., C₁-C₂₀haloalkyl, e.g., —CF₃, —CN, —NO₂, —OR^{c}, —NR^{c}R^{c}, C₁-C₂₀alkyl, C₂-C₂₀alkenyl, and C₂-C₂₀alkynyl;
each R^{c} is at each occurrence independently selected from H, C₁-C₂₀alkyl, C₂-C₂₀alkenyl, and C₂-C₂₀alkynyl.

In any of the embodiments of the process disclosed herein, R¹ and R² are each independently selected from H, C₁-C₂₀alkyl, C₃-C₁₀cycloalkyl, C₆-C₁₄aryl, C₇-C₂₀arylalkyl, 3-14 membered heterocyclyl, and 5-20 membered heteroaryl,
wherein the C₁-C₂₀alkyl is optionally substituted by one to six R^{a},
wherein the C₃-C₁₀cycloalkyl, C₆-C₁₄aryl, C₇-C₂₀arylalkyl, 3-14 membered heterocyclyl, and 5-20 membered heteroaryl are each optionally substituted by one to six R^{b},
each R^{a} is at each occurrence independently selected from halogen, e.g., F, C₁-C₂₀haloalkyl, e.g., —CF₃, —CN, —OR^{c}, and —NR^{c}R^{c};
each R^{b} is at each occurrence independently selected from halogen, e.g., F, haloalkyl, e.g., C₁-C₂₀haloalkyl, e.g., —CF₃, —CN, —NO₂, —OR^{c}, and —NR^{c}R^{c};
wherein each R^{c} is at each occurrence independently selected from H, and C₁-C₂₀alkyl.

In any of the embodiments of the process disclosed herein, R¹ is selected from hydrogen, and C₁-C₂₀alkyl, wherein the C₁-C₂₀alkyl is optionally substituted by one to six R^{a}, and R² is C₁-C₂₀alkyl, wherein the C₁-C₂₀alkyl is optionally substituted by one to six R^{a},
wherein each R^{a} is at each occurrence independently selected from halogen, e.g., F, and C₁-C₂₀haloalkyl, e.g., C₁-C₂₀fluoroalkyl, e.g., —CF₃.

In any of the embodiments of the process disclosed herein, at least one of R¹ and R² is C₁-C₂₀fluoroalkyl, e.g., C₁-C₆fluoroalkyl.

In any of the embodiments of the process disclosed herein, R¹ is selected from hydrogen, and C₁-C₆alkyl, wherein the C₁-C₆alkyl is optionally substituted by one to six F, and R² is C₁-C₆alkyl or phenyl.

In any of the embodiments of the process disclosed herein, R¹ is selected from hydrogen, and trifluoromethyl, and R² is methyl or phenyl.

In any of the embodiments of the process disclosed herein, R³ and R⁴ are each independently selected from H and C₁-C₂₀alkyl, wherein the C₁-C₂₀alkyl is optionally substituted by one to six R^{a},
wherein each R^{a} is at each occurrence independently selected from C₃-C₁₀cycloalkyl, C₆-C₁₄aryl, 3-14 membered heterocyclyl, 5-20 membered heteroaryl, halogen, e.g., F, haloalkyl, e.g., C₁-C₂₀haloalkyl, e.g., —CF₃, —OR^{c}, and —NR^{c}R^{c};
wherein each R^{c} is at each occurrence independently selected from H, C₁-C₂₀alkyl, C₂-C₂₀alkenyl, and C₂-C₂₀alkynyl.

In any of the embodiments of the process disclosed herein, R³ and R⁴ are each independently selected from H and C₁-C₂₀alkyl, wherein the C₁-C₂₀alkyl is optionally substituted by one to six R^{a},
wherein each R^{a} is at each occurrence independently selected from halogen, e.g., F, C₁-C₂₀haloalkyl, e.g., —CF₃, —OR^{c}, and —NR^{c}R^{c};
wherein each R^{c} is at each occurrence independently selected from H, C₁-C₂₀alkyl, C₂-C₂₀alkenyl, and C₂-C₂₀alkynyl.

In any of the embodiments of the process disclosed herein, R³ and R⁴ are each independently selected from H and C₁-C₂₀alkyl, wherein the C₁-C₂₀alkyl is optionally substituted by one to six R^{a},
wherein each R^{a} is at each occurrence independently selected from halogen, e.g., F, C₁-C₂₀haloalkyl, e.g., —CF₃, —OR^{c}, and —NR^{c}R^{c};
wherein each R^{c} is at each occurrence independently selected from H, and C₁-C₂₀alkyl.

In any of the embodiments of the process disclosed herein, the substrate is a ketone.

In any of the embodiments of the process disclosed herein, the nitroalkane substrate is nitromethane or nitroethane.

In any of the embodiments of the process disclosed herein, the ketone substrate is

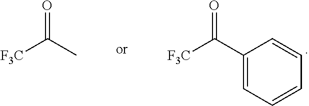

The present disclosure also provides a process for the asymmetric synthesis of (S)-1,1,1-trifluoro-2-methyl-3-nitropropan-2-ol (IA):

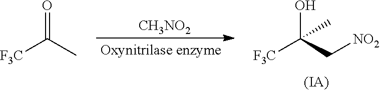

the process comprising the step of contacting nitromethane and 1,1,1-trifluoropropan-2-one with the oxynitrilase polypeptide according to the present disclosure, to obtain (S)-1,1,1-trifluoro-2-methyl-3-nitropropan-2-ol of formula (IA).

As disclosed herein and exemplified in the examples, the present disclosure contemplates a range of suitable reaction conditions that may be used in the process herein, including but not limited to pH, temperature, buffers, solvent systems, substrate loadings, mixtures of product stereoisomers, e.g., enantiomers, polypeptide loading, cofactor loading, pressure, and reaction time. Additional suitable reaction conditions for performing a method of enzymatically converting substrate compounds to a product compound using engineered oxynitrilase polypeptides described herein can be readily optimized by routine experimentation, which including but not limited to that the engineered oxynitrilase polypeptide is contacted with substrate compounds under experimental reaction conditions of varying concentration, pH, temperature, solvent conditions, and the product compound is detected, for example, using the methods described in the Examples provided herein.

As described above, engineered polypeptides having oxynitrilase activity for use in the process of the present disclosure generally comprises amino acid sequences that have at least 80%, 85%, 86%, 87%, 88%, 89%, 90%, 91%, 92%, 93%, 94%, 95%, 96%, 97%, 98% or 99% sequence identity to the reference amino acid sequence selected from any one of the even numbered sequences of SEQ ID NO: 4 to 604, and 608 to 640.

The substrate compounds in the reaction mixture can be varied, taking into consideration of, for example, the amount of the desired product compound, the effect of the substrate concentration on the enzyme activity, the stability of the enzyme under the reaction conditions, and the percent conversion of substrate to product. In any of the embodiments of the process disclosed herein, the suitable reaction conditions include at least about 0.5 to about 200 g/L, about 1 to about 200 g/L, about 5 to about 150 g/L, about 10 to about 150 g/L, or about 50 to about 150 g/L of loading of aldehyde or ketone substrate, e.g., formula (III). In an embodiment, suitable reaction conditions include at least about 0.5 g/L, at least about 1 g/L, at least about 5 g/L, at least about 10 g/L, at least about 15 g/L, at least about 20 g/L, at least about 30 g/L, at least about 50 g/L, at least about 75 g/L, at least about 100 g/L or even more of loading of aldehyde or ketone substrate, e.g., formula (III). The values for the substrate loading provided herein are based on the molecular weight of the aldehyde or ketone substrate, e.g., formula (III), however it is also contemplated that the equivalent molar amounts of various hydrates and salts of the aldehyde or ketone substrate, e.g., formula (III), may also be used in the process.

In any of the embodiments of the process disclosed herein, the engineered oxynitrilase polypeptides use aldehyde or ketone substrate and a nitroalkane compound to form a β-nitro alcohol product compound. In an embodiment, suitable reaction conditions include nitroalkane present in a loading of at least about 1 times of the molar loading of the aldehyde or ketone substrate, e.g., formula (III). In an embodiment, the nitroalkane is present at a loading of 2, 3, 4, 5, 6, 7, 8, 9 or 10 times of the molar loading of the aldehyde or ketone substrate, e.g., formula (III).

In any of the embodiments of the process disclosed herein, suitable reaction conditions include nitroalkane present in a loading of 0.5 to about 200 g/L, about 1 to about 200 g/L, about to about 150 g/L, about 10 to about 150 g/L, or about 50 to about 150 g/L of loading of nitroalkane substrate. In an embodiment, suitable reaction conditions include at least about 0.5 g/L, at least about 1 g/L, at least about 5 g/L, at least about 10 g/L, at least about 15 g/L, at least about 20 g/L, at least about 30 g/L, at least about 50 g/L, at least about 75 g/L, at least about 100 g/L or even more of loading of nitroalkane substrate.

In any of the embodiments of the process disclosed herein, the reaction conditions may include a suitable pH. As noted above, the desired pH or desired pH range can be maintained by using an acid or base, a suitable buffer, or a combination of buffer and added acid or base. The pH of the reaction mixture can be controlled before and/or during the reaction. In some embodiments, suitable reaction conditions include a solution pH of about 4 to about 8, a pH of about 5 to about 7, a pH of about 6 to about 7. In some embodiments, the reaction conditions include a solution pH of about 4, 4.5, 5, 5.5, 6, 6.5, 7, 7.5 or 8.

In any of the embodiments of the process disclosed herein, suitable temperatures can be used for the reaction conditions, taking into consideration of, for example, the increase in reaction rate at higher temperatures, the activity of the enzyme for sufficient duration of the reaction. Accordingly, in some embodiments, suitable reaction conditions include a temperature of about 10° C. to about 60° C., about 25° C. to about 50° C., about 25° C. to about 40° C., about 25° C. to about 30° C., or about 10° C. to about 30° C. In some embodiments, suitable reaction temperatures include a temperature of about 10° C., 15° C., 20° C., 25° C., 30° C., 35° C., 40° C., 45° C., 50° C., 55° C., or 60° C. In some embodiments, the temperature during the enzymatic reaction can be maintained at a certain temperature throughout the reaction. In some embodiments, the temperature during the enzymatic reaction may be adjusted over a temperature profile during the course of the reaction.

The processes of using the engineered oxynitrilases are generally carried out in a solvent. However, the processes may also be carried out in the absence of a solvent.

Suitable solvents include water, aqueous buffer solutions, organic solvents, and/or co-solvent systems, which generally include aqueous solvents and organic solvents. The organic solvent may be any organic solvent. Preferably, it shall not disturb or inhibit the enzymatic reaction. In certain embodiments, the organic solvent is water-miscible or partly water-miscible. The organic solvent especially is an aprotic organic solvent.

The aqueous solutions (water or aqueous co-solvent systems) can be pH-buffered or unbuffered. In some embodiments, the processes of using an engineered oxynitrilase polypeptide are generally carried out in solvent system comprising an organic solvent. In some embodiments, the processes of using an engineered oxynitrilase polypeptide are generally carried out in an aqueous co-solvent system comprising an organic solvent.

In any of the embodiments of the process disclosed herein, the organic solvent is selected from methanol, ethanol, propanol, isopropanol, dimethyl sulfoxide (DMSO), dimethylformamide (DMF), isopropyl acetate, ethyl acetate, butyl acetate, 1-octanol, hexane, heptane, octane, methyl tert-butyl ether (MTBE), toluene, benzene, glycerol, polyethylene glycol, and an ionic liquid, e.g., 1-ethyl 4-methylimidazolium tetrafluoroborate, 1-butyl-3-methylimidazolium tetrafluoroborate, 1-butyl-3-methylimidazolium hexafluorophosphate.

The organic solvent component of the aqueous co-solvent system may be miscible with the aqueous component, providing a single liquid phase, or may be partially miscible or immiscible with the aqueous component, providing two liquid phases. Exemplary aqueous co-solvent system comprises water and one or more organic solvents as defined herein. In general, the organic solvent component of the aqueous co-solvent system is selected such that it does not completely inactivate the oxynitrilase. Suitable co-solvent systems can be readily identified by measuring the enzymatic activity of a particular engineered oxynitrilase with a defined substrate of interest in the candidate solvent system, utilizing enzymatic activity assays, such as those described herein. In any of the embodiments of the process disclosed herein, suitable reaction conditions include an aqueous co-solvent system comprising isopropyl acetate at a concentration of about 1% to about 60% (v/v), about 1% to about 50% (v/v), about 1% to about 40% (v/v), about 2% to about 40% (v/v), about 5% to about 40% (v/v), from about 10% to about 40% (v/v), from about 10% to about 30% (v/v), or about 10% to about 20% (v/v). In some embodiments of the process, suitable reaction conditions include an aqueous co-solvent system comprising isopropyl acetate at a concentration of at least about 1%, 5%, 10%, 15%, 20%, 25%, 30%, 35%, 40%, 45%, 50%, 55%, or 60% (v/v).

Suitable reaction conditions can include a combination of reaction parameters that provide for the biocatalytic conversion of the substrate compounds to its corresponding product compound. Accordingly, in some embodiments of the process, the combination of reaction parameters comprises: (a) substrate loading, e.g., 1,1,1-trifluoropropan-2-one loading of about 5 g/L to about 150 g/L; (b) nitroalkane loading, e.g., nitromethane loading is about 2 times the molar amount of the substrate, e.g., 1,1,1-trifluoropropan-2-one; (c) engineered polypeptide concentration of about at least 3 g/L; (d) aqueous isopropyl acetate concentration of about 1% (v/v) to about 60% (v/v); (e) pH of about 4.0 to 8.0; and (f) temperature of about 10° C. to 30° C.

Exemplary reaction conditions include the assay conditions provided in the Examples section.

In carrying out the reaction described herein, the engineered oxynitrilase polypeptide biocatalyst may be added to the reaction mixture in different formulation forms, as frozen or lyophilized whole cells (FWC or LWC) transformed with the gene encoding the engineered oxynitrilase polypeptide and/or as cell lysate or lyophilized cell lysate of such cells, so called shake flask powder (SFP), where the cell debris was removed and/or further purified as fermentation powder (FP). Whole cells transformed with the gene encoding the engineered oxynitrilase or cell extracts, lysates thereof, and isolated enzymes can be used in a wide variety of different forms, including solids (e.g., lyophilized, spray dried, or the like) or semisolid (e.g., a crude paste). The cell extract or cell lysate may be partially purified by precipitation (e.g., ammonium sulfate, polyethyleneimine, heat treatment or the like), followed by desalting procedures (e.g., ultrafiltration, dialysis, and the like) prior to lyophilization. Any of the enzyme preparations can be stabilized by crosslinking using known crosslinking agents, such as glutaraldehyde, or immobilization to a solid phase material (such as a resin).

In any of the embodiments of the process disclosed herein, the reaction is performed under suitable reaction conditions described herein, wherein the engineered oxynitrilase polypeptide is immobilized to a solid support, such as a membrane, resin, solid carrier, or other solid phase material. A solid support can be composed of organic polymers such as polystyrene, polyethylene, polypropylene, polyfluoroethylene, polyethyleneoxy, polymethacrylate, and polyacrylamide, as well as co-polymers and grafts thereof. A solid support can also be inorganic, such as glass, silica, controlled pore glass (CPG), reverse phase silica or metal, such as gold or platinum. The configuration of a solid support can be in the form of beads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression, or other container, vessel, feature, or location. Solid supports useful for immobilizing the engineered oxynitrilase enzyme for carrying out the reaction include but are not limited to beads or resins such as polymethacrylate, e.g., polymethacrylates with epoxy functional groups, polymethacrylates with amino epoxy functional groups, polymethacrylates, styrene/DVB copolymer or polymethacrylates with octadecyl functional groups.

In a particular embodiment, the solid support is a bead or resin comprising polymethacrylate.

Exemplary solid supports include, but are not limited to, chitosan beads, Eupergit C, IB-150, IB-350, IB-C435, IB-A369, IB-A161, IB-A171, IBS500, IB-S861, SEPABEADS (Mitsubishi), e.g., Sepabeads EC-EP, Sepabeads EC-HFA, Sepabeads EC-HG, Sepabeads EC-BU, Sepabeads EC-OD, Sepabeads EC-CM, Sepabeads EC-IDA, Sepabeads EC-EA, Sepabeads EC-HA, Sepabeads EC-QA, Sepabeads EXE, Sepabeads EXA, Dilbeads-TA, Amberzyme Oxirane, Amberlite XAD-7HP, Amberlite FPA98Cl, Amberlite IRA958Cl, Amberlite IRA67, Amberlite FPA90Cl, Amberlite FPA40Cl, Amberlite XAD18, Accurel EP100, ECR8206F/5730, ECR8206/5803, ECR8206M/5749, ReliZyme EP403, ReliZymeEP113, Lewatit VP OC 1600, Diaion WA20, Diaion WA21J, Diaion WA30, Dowex 66, Diaion HPA-25L, Lewatit VP OC 1064 MD PH, Lewatit VP OC 1163, Lifetech ECR8304F. Lifetech ECR8309F, Lifetech ECR8315F, Lifetech ECR8204F, Lifetech ECR8285, Lifetech ECR1090M, Lifetech ECR1030M, Lifetech ECR8806M, Chromalite (MAM2/F) D6591, Chromalite MIDA/M, Chromalite MIDA/M/Fe, Chromalite MIDA/M/Co, Chromalite MIDA/M/Ni, Chromalite MIDA/M/Cu and Chromalite MIDA/M/Zn.

In any of the embodiments of the process disclosed herein, wherein an engineered polypeptide is expressed in the form of a secreted polypeptide, a culture medium containing the secreted polypeptide can be used in the process herein.

In any of the embodiments of the process disclosed herein, the solid reactants (e.g., enzymes, salts, etc.) can be provided to the reaction in a variety of different forms, including powders (e.g., lyophilized, spray dried, etc.), solutions, emulsions, suspensions, and the like. The reactants can be readily lyophilized or spray-dried using methods and instrumentation known to one skilled in the art. For example, the protein solution can be frozen at −80° C. in small aliquots, and then added to the pre-chilled lyophilization chamber, followed by the application of a vacuum.

In any of the embodiments of the process disclosed herein, the order of addition of reactants is not critical. The reactants may be added together to the solvent at the same time (e.g., monophasic solvent, a biphasic aqueous co-solvent system, etc.), or alternatively, some reactants may be added separately, and some may be added together at different time points. For example, the oxynitrilase and substrates may be added first to the solvent. For improved mixing efficiency when using aqueous co-solvent systems, oxynitrilase may be added and mixed into the aqueous phase first. The organic phase, e.g., isopropyl acetate, can then be added and mixed in, followed by addition of the substrates. Alternatively, the substrates can be premixed in the organic phase prior to addition to the aqueous phase.

The methods of performing an enzymatic reaction may comprise the further step of isolating the product of the enzymatic reaction. In particular, this step is performed after completion of the enzymatic reaction. The product is in particular separated from one or more, in particular essentially all of the other components of the reaction mixture. For example, the product is separated from the remaining substrate, side products, the enzyme, and/or organic solvents. Isolation of the product may be achieved by means and techniques known in the art, including for example evaporation of solvents, aggregation or crystallization and filtration, phase separation, chromatographic separation and others.

An engineered oxynitrilase polypeptide that can be used in any of the embodiments of the process disclosed herein can comprise one or more sequences selected from the amino acid sequences corresponding to SEQ ID NOs: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 608, 610, 612, 614, 616, 618, 620, 622, 624, 626, 628, 630, 632, 634, 636, 638, or 640.

In any of the embodiments of the processes disclosed herein, the β-nitro alcohol product, e.g., formula (IA), is obtained with at least 55% ee, e.g., at least 65% ee, e.g., at least 75%, e.g., at least 80%, e.g., at least 90% ee, e.g., at least 95% ee, e.g., at least 99%, enantiomeric excess (e.e.).

In any of the embodiments of the process disclosed herein, the β-nitro alcohol compound, e.g., formula (IA), is obtained with a conversion rate of at least 10%, e.g., at least 20%, e.g., at least 30% e.g., at least 40%, e.g., at least 50%, more preferably with at least 75%, at least 85%, at least 90%, e.g., 95%.

The present disclosure also provides a process for synthesizing (S)-3-amino-1,1,1-trifluoro-2-methylpropan-2-ol of formula (IB):

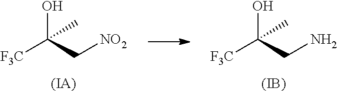

the process comprising the step of contacting (IA) with hydrogen under suitable hydrogenation conditions, to obtain (S)-3-amino-1,1,1-trifluoro-2-methylpropan-2-ol (IB), wherein (IA) is synthesized by the process according to according to the present disclosure.

The source of hydrogen can be selected from gaseous hydrogen (H₂), hydrogen donors (transfer hydrogenation, e.g., formic acid or salts thereof), hydride reagent (LiAlH₄) or the like.

Suitable hydrogenation reaction conditions include the presence of hydrogen gas, a transition metal catalyst, and a suitable solvent. In an embodiment, the transition metal catalyst is selected from sponge metal, e.g., sponge nickel, sponge cobalt, Pd/C, Pt/C, PtO₂, Rh/Al₂O₃ and Pd/BaSO₄. In particular, the catalyst is sponge nickel. In an embodiment, the transition metal catalyst is present at a loading of at least 2 wt. %, e.g., at least 5 wt. %, at least 10 wt. %. The sponge metal or Raney metal catalysts which may be employed in the reaction contain from 0.1 to 10%, for example, about 0.5 to 3%, by weight molybdenum based upon the weight of the catalyst, as a promoter. Sponge nickel promoted with molybdenum is the preferred catalyst, although sponge cobalt or Raney cobalt is well suited. These catalysts may be employed in the reaction medium at a loading of at least 2 wt. %, e.g., at least 5 wt. %, at least 10 wt. %. In an embodiment, the transition metal catalyst is metal promoted sponge nickel, e.g., molybdenum promoted sponge nickel. In an embodiment, the transition metal catalyst is unpromoted sponge nickel. In an embodiment, the solvent is selected from water, ethanol, methanol, n-propanol, isopropanol, ethyl acetate, isopopyl acetate, butyl acetate, tert-butyl methyl ether, and tetrahydrofuran. In an embodiment, the reaction is carried out at a temperature of about 20-60° C., e.g., 20° C., 25° C., 30° C., 35° C., 40° C., 45° C., 50° C., 55° C., 60° C. In an embodiment, the reaction is carried out at a pressure of about 0.1-40 bar. In an embodiment, the compound of formula (IA) is present at a concentration of at least 2 wt. %, e.g., 2, 4, 8, 10 wt. %. In an embodiment, the hydrogenation reaction is carried out carried out in batch or flow.

In a further embodiment, the process described above for producing (IB) further comprises the step of converting the compound of formula (IB) to an acid salt. In a further embodiment, the process comprises the additional step of crystallizing the acid salt. Preferably, the acid salt is a HCl salt. The crystallization step increases the enantiomeric purity of compound (IB). Advantageously, the compound (IB) having an ee of about 92% before isolation increased to around 98% after isolation of the crystallized compound (IB) HCl salt.

The present disclosure also provides a process for synthesizing (S)-3-amino-6-methoxy-N-(3,3,3-trifluoro-2-hydroxy-2-methylpropyl)-5-(trifluoromethyl)picolinamide of formula (IC)

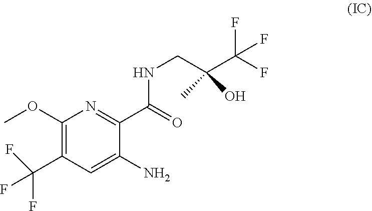 [SMILES: COc1nc(C(=O)NC[C@](C)(O)C(F)(F)F)c(N)cc1C(F)(F)F]

the process comprising the step of contacting nitromethane and 1,1,1-trifluoropropan-2-one with the oxynitrilase polypeptide according to the present disclosure, to obtain (S)-1,1,1-trifluoro-2-methyl-3-nitropropan-2-ol of formula (IA).

In a particular embodiment, the process further comprises the step of contacting the compound of (IA) with hydrogen under suitable hydrogenation conditions, to obtain (S)-3-amino-1,1,1-trifluoro-2-methylpropan-2-ol (IB). In a further embodiment, the process further comprises the step of converting the compound of formula (IB) to an acid salt. Preferably, the acid salt is a HCl salt (compound (IB). HCl).

In a further embodiment, the process comprises the additional step of crystallizing the acid salt.

The further steps required to synthesize the compound of formula (IC) from (IB) or (IB). HCl may be employed according to the disclosure of EP3555048 B1, e.g., according to Scheme and [0028] and the Examples therein.

Alternatively, the process may further comprise the step of coupling the compound of formula (IB). HCl or crystallized (IB). HCl with a compound of formula (E6) in the presence of a coupling reagent under suitable reactions conditions,

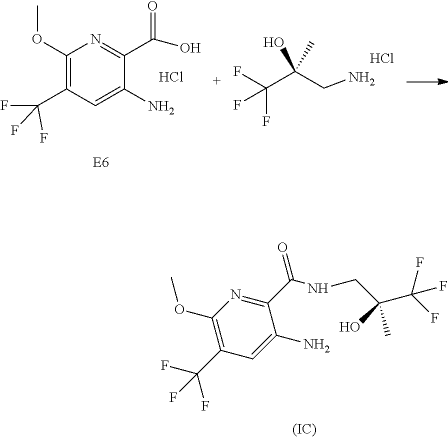

to produce a compound of formula (IC).

Suitable reaction conditions can be those generally employed in the amidation of carboxylic acids, as known in the art. These may include a suitable coupling reagent such as tetramethyl orthosilicate (TMOS), N-(3-dimethylaminopropyl)-N′-ethylcarbodiimide hydrochloride (EDC), dicyclohexylcarbodiimide (DCC), 1,1′-carbonyldiimidazole (CDI), e.g., in the presence of a suitable base, such as Et₃N, DIEA, DMAP or pyridine, in a suitable solvent, such as THF, DCM or toluene, at a suitable temperature, such as between 0° C. and 120° C. In a particular embodiment, the coupling reagent is tetramethyl orthosilicate. In a particular embodiment, suitable reaction conditions include Et₃N, toluene and heating the reaction to about 110° C.

The full procedure for the synthesis of compound (IC) is described in Scheme 2 vide infra.

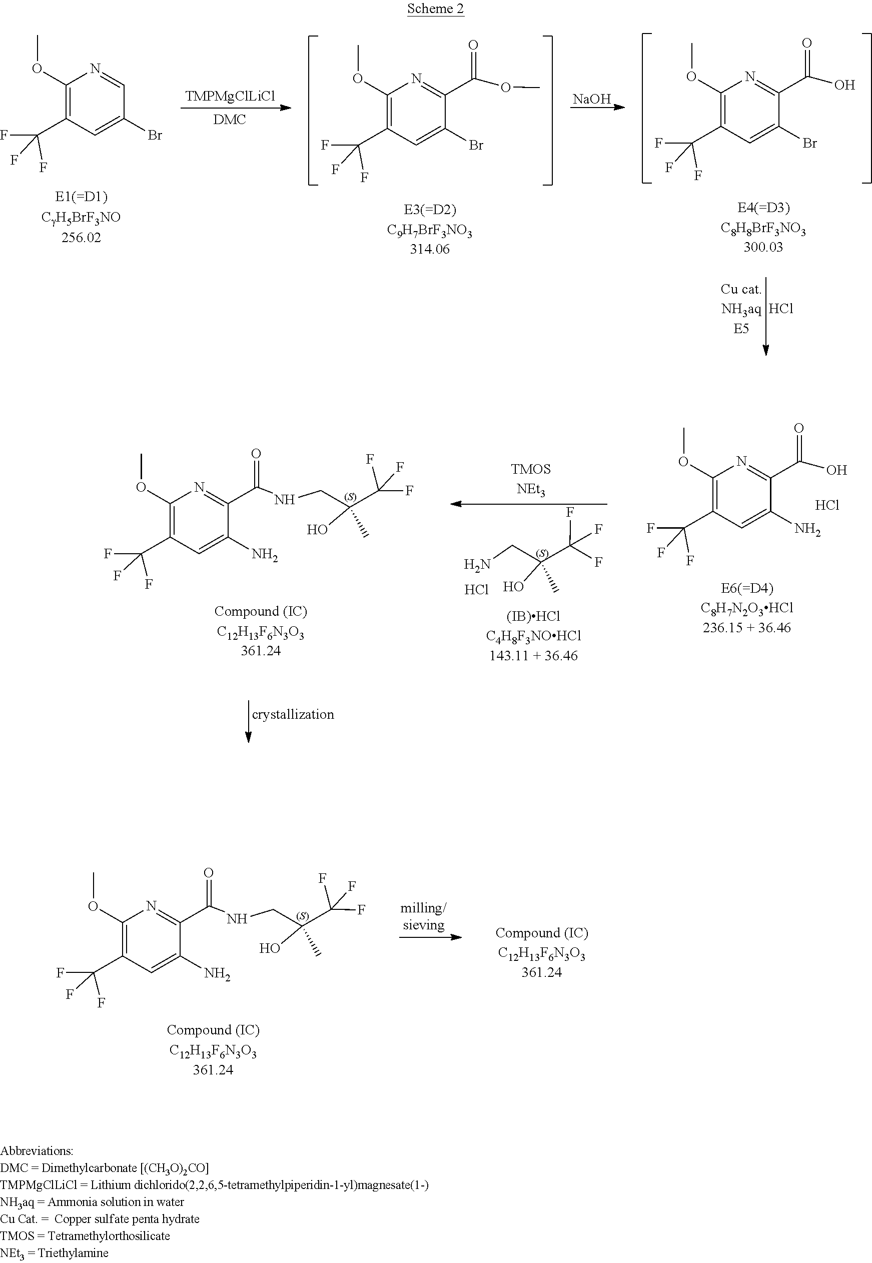

In a further embodiment, there is provided a process for the preparation of a compound of formula (IC)

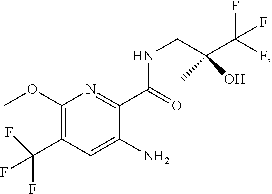 [SMILES: COc1nc(C(=O)NC[C@](C)(O)C(F)(F)F)c(N)cc1C(F)(F)F]

in free form or in pharmaceutically acceptable salt form according to Scheme 2. In a further embodiment of Scheme 2, compound (IB).HCl is synthesised according to the present disclosure.

Different features and embodiments of the present disclosure are exemplified in the following representative examples, which are intended to be illustrative and not restrictive.

### EXAMPLES

The following Examples, including experiments and results achieved, are provided for illustrative purposes only and are not to be construed as limiting the present invention.

In the Examples below, the following abbreviations apply: ppm (parts per million); M (molar); mM (millimolar), uM and μM (micromolar); nM (nanomolar); mol (moles); gm and g (gram); mg (milligrams); ug and μg (micrograms); L and 1 (liter); ml and mL (milliliter); cm (centimeters); mm (millimeters); um and μm (micrometers); sec. (seconds); min(s) (minute(s)); h(s) and hr(s) (hour(s)); U (units); MW (molecular weight); rpm (rotations per minute); psi and PSI (pounds per square inch); ° C. (degrees Centigrade); RT and rt (room temperature); OD600 (Optical density at 600 nm), CAM and cam (chloramphenicol); IPAc (isopropylacetate), DMSO (dimethylsulfoxide); FP (Fermentation powder); FWC (Frozen whole cells), LWC (Lyophilized whole cells), PMBS (polymyxin B sulfate); IPTG (isopropyl β-D-1-thiogalactopyranoside); LB (Lysogeny broth); NM (Nitromethane), MeOH (Methanol), MTBE (Methyl-tert-butylether), TB (Terrific Broth; 12 g/L bacto-tryptone, 24 g/L yeast extract, 4 mL/L glycerol, 65 mM potassium phosphate, pH 7.0, 1 mM MgSO4); PLP (pyridoxal 5′-phosphate), TEoA (triethanolamine buffer), HEPES (HEPES zwitterionic buffer; 4-(2-hydroxyethyl)-piperazineethanesulfonic acid); SFP (shake flask powder); CDS (coding sequence); DNA (deoxyribonucleic acid); RNA (ribonucleic acid); E. coli W3110 (commonly used laboratory E. coli strain, available from the Coli Genetic Stock Center [CGSC], New Haven, CT); HTP (high throughput); HPLC (high pressure liquid chromatography); GC (gas chromatography), MS (mass spectrometer), RF (Rapid Fire), FIOP (fold improvements over positive control); Microfluidics (Microfluidics, Corp., Westwood, MA); Sigma-Aldrich (Sigma-Aldrich, St. Louis, MO; Difco (Difco Laboratories, BD Diagnostic Systems, Detroit, MI); Agilent (Agilent Technologies, Inc., Santa Clara, CA); Corning (Corning, Inc., Palo Alto, CA); Dow Corning (Dow Corning, Corp., Midland, MI); and Gene Oracle (Gene Oracle, Inc., Mountain View, CA).

Throughout the Examples, “T100% conv” corresponds to the calculated conversion for each single sample expressed in percentage with regard to the theoretical 100% conversion value. The amount of produced nitroaldol product in each single sample is quantified by HPLC using an external standard of nitroaldol product with a known concentration. The conversion of each single sample is obtained by dividing the calculated amount of the produced nitroaldol product by the theoretical amount of nitroaldol product when 100% conversion is achieved, then the obtained value is multiplied by 100, expressing the conversion result in percentage.

### Example 1

Production of Engineered Polypeptides in pCK110900

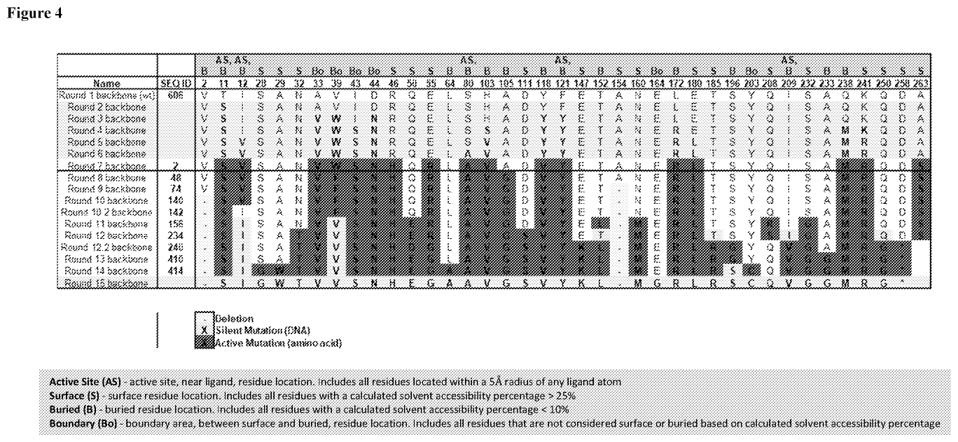

The polynucleotide (SEQ ID NO: 1) encoding the polypeptide having Oxynitrilase activity (SEQ ID NO: 2), was cloned into the pCK110900 vector system (See e.g., US Pat. App. No. 2006/0195947A1 FIG. 3 which is hereby incorporated by reference in its entirety) and subsequently expressed in E. coli W3110fhuA under the control of the lac promoter. The expression vector also contained the P15a origin of replication and the chloramphenicol (CAM) resistance gene. This polynucleotide, and associated polypeptide, was a product of six rounds of directed evolution starting from a (S)-hydroxynitrile lyase found in Baliospermum montanum (UniProt D1MX73).

### Example 2

### Preparation of Cell Pellets

E. coli W3110fhuA cells were transformed with the pCK110900 plasmid containing the oxynitrilase-encoding genes. Transformed cells were plated out on Lysogeny broth (LB) agar plates containing 1% glucose and 30 μg/mL CAM, and grown overnight at 37° C. Subsequently single colonies were picked in a 96-well format and grown in 190 μL LB media containing 1% glucose and 30 μg/mL CAM, at 30° C., 200 rpm, and 85% humidity. Following overnight growth, 20 μL of the grown cultures were transferred into a deep well plate containing 380 μL of Terrific Broth (TB) media with 30 μg/mL CAM. The cultures were grown at 30° C., 250 rpm, with 85% humidity for approximately 2.5 hours. When the optical density (OD600) of the cultures reached 0.4-0.8, expression of the oxynitrilase gene was induced by the addition of isopropyl-β-D-thiogalactoside (IPTG) to a final concentration of 1 mM. Following induction, growth continued for 18-20 hours at 30° C., 250 rpm with 85% humidity. Cells were harvested by centrifugation at 4,000 rpm and 4° C. for 10 minutes; the supernatant was then discarded. The cell pellets were stored at −80° C. until ready for use.

### Example 3

### Lysis and Preparation of Clarified Lysate

Prior to performing the assay, the cell pellets were thawed and resuspended in 300 μL of lysis buffer (containing 1 g/L lysozyme, 0.5 g/L PMBS and 0.1 μL/mL or 0.2 U/ml of commercial DNAse (New England BioLabs, M0303L) in 0.1M Citric buffer at pH 6.0. The plates were agitated with medium-speed shaking for 2.5 hours on a microtiter plate shaker at room temperature. The plates were then centrifuged at 4,000 rpm for 10 minutes at 4° C., and the clarified supernatants were used in the HTP assay reaction for activity determination as described in the following examples.

### Example 4

### Preparation of Frozen Whole Cells (FWC), Lyophilized Whole Cells (LWC), Shake Flask Powder (SFP) and Fermentation Powder (FP)

Shake-flask procedures can be used to generate engineered oxynitrilase polypeptide shake-flask powders (SFP), which are useful for secondary screening assays and/or use in the biocatalytic processes described herein. Shake flask powder preparation of enzymes provides a more concentrated preparation of the engineered enzyme, as compared to the cell lysate used in HTP assays. To start the culture, either a single colony from a plate or a glycerol stock of E. coli containing a plasmid encoding an engineered polypeptide of interest, was inoculated into 25 mL of LB supplemented with 30 μg/mL CAM and 1% glucose in a 250 ml baffled shake flask. The culture was grown overnight (16-20 hours and OD₆₀₀>3.8) in an incubator at 37° C., with shaking at 250 rpm. A 1 μL shake flask containing 250 mL of TB media with 30 μg/mL CAM, was inoculated with 5 mL of the grown overnight culture. The 250 mL culture was incubated at 30° C., 250 rpm, for 3-3.5 hours until OD₆₀₀ reached 0.6-0.8. Expression of the oxynitrilase gene was induced by the addition of IPTG to a final concentration of 1 mM, and growth was continued for an additional 18-20 hours. Cells were harvested by transferring the culture into a centrifuge bottle, which was then centrifuged at 7,000 rpm for 5 minutes at 4° C. The supernatant was discarded, and the remaining cell pellet was either lysed or in some embodiments, stored at −80° C. as frozen whole cells (FWC) until ready to use. For lysis, the cell pellet was resuspended in 30 mL of 50 mM Citrate buffer at pH 6.0 and lysed using a LM20 MICROFLUIDIZER© processor system (Microfluidics). Cell debris was removed by centrifugation at 14,000 rpm for 30 minutes at 4° C. The clarified lysate was collected, frozen at −80° C., and then lyophilized, using standard methods known in the art. Lyophilization of frozen clarified lysate provides a dry shake-flask powder (SFP) and lyophilization of FWCs provides dry lyophilized whole cells (LWC), both comprising crude engineered oxynitrilase polypeptide. To access larger quantities of enzymes, which are necessary for upscaled reactions like in example 12, cells were fermented in a bioreactor using standard methods known in the art. Compared to SFP this fermentation powder (FP) had a higher concentration of the engineered oxynitrilase polypeptide due to a lower salt content.

### Example 5

### Analytical Method for Activity and Selectivity Evaluation

Activity improvements of the engineered oxynitrilases were analyzed either by High Pressure Liquid Chromatography (HIPLC) using the method described in Table 5.1 or RapidFire® Mass Spectrometry (RF-MS) using the method described in Table 5.2. HIPLC method with UV-detection was developed to analyze the conversion of compound (1) and (2) to compounds (IA). Only the nitromethane substrate (2) and the nitro alcohol product (IA) could be detected without any differentiation between (IA) vs. (R) enantiomer of (IA). In Examples 9, 10 and 11, RF-MS was used for rapid quantification of the product (IA). The corresponding method parameters are described in Table 5.2.

TABLE 5.1
HPLC method used for activity determination.

| Instrument | Agilent Technologies 1290 Infinity II with | | |
|---|---|---|---|
| | UV detection | | |
| Column | Kinetex ®2.6 μm EVO C18 100 Å | | |
| | (PN: 00B-4725-AN) Length 50 mm, | | |
| | internal diameter 2.1 mm or equivalent | | |
| | column, particle size: 2.6 μm. | | |
| Mobile Phase | A: 5 mM Potassium Phosphate Buffer, | | |
| | pH 6.3. | | |
| | B: Methanol | | |
| | | | Flowrate |
| | Time (min.) | Phase B (%) | (mL/min) |
| Gradient | 0 | 2 | 0.6 |
| | 0.2 | 2 | 0.6 |
| | 0.8 | 80 | 1.2 |
| | 1.0 | 80 | 0.6 |
| | 1.10 | 2 | 0.6 |
| | 1.70 | 2 | 0.6 |

| | |
|---|---|
| Run Time | 1.70 minutes |
| Compound elution | Nitromethane substrate (2): 0.27 min. |
| | Nitroaldol product (IA): 0.73 min |
| Column Temperature | 40° C. |
| Autosampler Temperature | 15° C. |
| Injection volume | 1 μL |
| Detection | UV 220 nm |

TABLE 5.2
RF-MS method used for activity determination

| | |
|---|---|
| Instrument | Agilent RapidFire 365 high throughput with a 6470 |
| | triple quadrupole mass spectrometer (MS-TQ) |
| Cartridge | RapidFire Cartridge, graphitic carbon (type D) |
| | G9206A (Agilent) |
| | Size: 4 μL |
| Mobile Phase | A: Water/Acetonitrile/Formic Acid (FA) |
| | (98.5:1.5:0.10/%) |
| | B: Water/Acetonitrile/FA (5:95:0.10/%) |
| | C: Water/Acetonitrile/FA (5:95:0.10/%) |
| MS source | Gas Temperature: 230° C. |
| parameters | Gas flow: 5 L/min |
| | Nebulizer: 40 psi |
| | Sheath Gas Temperature: 250° C. |
| | Sheath Gas Flow: 12 L/min |
| | Capillary: 5000 V (NEG) |
| | Nozzle Voltage: 1500 V (NEG) |
| MS detection | MRM mod |
| parameters | Polarity: Negative |
| | Precursor Ion: 172 |
| | Product ion one: 102 |
| | Product ion two: 69 |
| | For both: Dwell time 100 ms, Fragmentor 120 and |
| | Collision energy 20 |

The Selectivity towards either the desired (S′)-(IA) or the undesired (R)-(IA) of selected samples were analyzed by gas chromatography (GC) using the method described in Table 5.3.

TABLE 5.3
GC method used for selectivity determination.

| Instrument | Agilent 8890 GC with FID detection | | |
|---|---|---|---|
| Column | Chiraldex ® G-TA. | | |
| | Length 30 mm, internal diameter 0.250 mm or | | |
| | equivalent column, film thickness: 0.12 μm. | | |
| Detector parameters | Temperature: 250° C. | | |
| | Flow H2: 30 mL/min | | |
| | Air flow: 400 mL/min | | |
| | Makeup (N2): 25 mL/min | | |
| Injector parameters | Temperature: 250° C. | | |
| | Split ratio: 50 | | |
| Carrier gas | Helium (constant 5 ml/min) | | |
| | Time (min) | Temperature (° C.) | Rate (° C./min) |
| Gradient | 0 | 100 | 0 |
| | 0.5 | 100 | 2.5 |
| | 4.5 | 110 | 0 |
| | 5.0 | 110 | Next injection |

| | |
|---|---|
| Run Time | 5.00 minutes |
| Compound elution | product R-enantiomer of (IA): 3.7 min |
| | product (IA): 4.1 min |
| Injection volume | 1 μL |

In Examples 20 the LC-MS was used for the detection of the products 1,1,1-trifluoro-2-methyl-3-nitrobutan-2-ol (4) and 1,1,1-trifluoro-3-nitro-2-phenylpropan-2-ol (8). The method parameters are described in Table 5.4.

TABLE 5.4
LC-MS method used for product detection.

| Instrument | Agilent Technologies 1290 Infinity II with MS | |
|---|---|---|
| | detection (Ultivo) | |
| Column | Waters Acquity BEH C18 Length 50 mm, | |
| | internal diameter 2.1 mm or equivalent column, | |
| | particle size: 1.7 μm. | |
| Mobile Phase | A: Water/Acetonitrile (90:10 v/v) + 0.1% formic acid | |
| | B: Water/Acetonitrile (10:90 v/v) + 0.1% formic acid | |
| | Time (min.) | Phase B (%) |
| Gradient | 0 | 10 |
| | 1.75 | 95 |
| | 2.00 | 95 |
| | 2.10 | 10 |
| | 3.00 | 10 |

| | |
|---|---|
| Flow | 0.750 ml/min. |
| Run Time | 3.00 minutes |
| Column | 40° C. |
| Temperature | |
| Autosampler | 15° C. |
| Temperature | |
| Injection volume | 1 μL |
| Detection | UV 220 nm |
| MS detection | Ion source: AJS ESI |
| | Detection mode: Scan (50 ms), Negative |
| | Fragmentor: 110 V |
| | Gas temperature: 250° C. |
| | Gas flow: 11.0 L/min |
| | Nebulizer: 30 psi |
| | Sheath gas temperature: 250° C. |
| | Sheath gas flow: 11.0 l/min |
| | Capillary voltage: 4500 V |
| | Nozzle voltage: 1500 V |

In Examples 20 the GC-MS/FD was used for the detection of the product 1-nitro-2-phenylpropan-2-ol (6). The method parameters are described in Table 5.5.

TABLE 5.5
GC-MS/FID method used for product detection.

| Instrument | Agilent 8890 GC with FID detection and MS | | |
|---|---|---|---|
| | (SQ) detection | | |
| Column | Agilent DB-XLB | | |
| | Length 30 mm, internal diameter 0.250 mm or | | |
| | equivalent column, film thickness: 1.0 μm. | | |
| Detector | Temperature: 300° C. | | |
| parameters | Flow H2: 30 mL/min | | |
| | Air flow: 400 mL/min | | |
| | Makeup (N2): 25 mL/min | | |
| Injector | Temperature: 300° C. | | |
| parameters | Split ratio: 200 | | |
| Carrier gas | Helium (constant 1.2 ml/min) | | |
| | Time (min) | Temperature (° C.) | Rate (° C./min) |
| Gradient | 0 | 50 | 0 |
| | 0.5 | 50 | 20 |
| | 13.0 | 300 | 0 |
| | 13.5 | 300 | Next injection |

| | |
|---|---|
| Run Time | 13.5 minutes |
| Injection volume | 1 μL |
| MS Parameters | EI-SCAN |
| Temperature | MS Source 230° C. |
| | MS Quand 150° C. |
| Detector | Low mass 30 |
| | High Mass 600 |
| | Threshold 4 |

The methods provided herein find use in analyzing the variants produced using the present invention. However, it is not intended that present invention be limited to the methods described herein, as there are other suitable methods known in the art that are applicable to the analysis of the variants provided herein and/or produced using the methods provided herein.

### Example 6

Round 7 Evolution and Screening of Engineered Polypeptides Derived from SEQ ID NO: 2 for Improved Production of Compound (IA)

The engineered polynucleotide (SEQ TD NO: 1) encoding the polypeptide with oxynitrilase activity of SEQ TD NO: 2 was used to generate the engineered polypeptides of Table 6-1. These polypeptides displayed improved oxynitrilase activity under the desired conditions e.g., the improvement in the formation of the nitro alcohol compound (IA), that was produced in situ from the substrates trifluoroacetone and nitromethane, compounds (1) and (2), respectively, as compared to the starting polypeptide. Some polypeptides displayed improved product formation of nitro alcohol product (IA) compared to the starting polypeptide are noted in Table 6-1. The engineered polypeptides, having the amino acid sequences of even-numbered sequence identifiers were generated from the “backbone” amino acid sequence of SEQ ID NO: 2, as described below together with the analytical method described in Table 5-1.

Directed evolution began with the polynucleotide set forth in SEQ ID NO: 1. Libraries of engineered polypeptides were generated using various well-known techniques (e.g., saturation mutagenesis, recombination of previously identified beneficial amino acid differences) and screened using HTP assay and analysis methods, described below, that measured the polypeptides' ability to produce compound (IA).

The enzyme assays were carried out in 96-well deep-well (1.1 mL total volume) plates, in 100 μL total reaction volume per well. The reactions contained 91 v/v % of undiluted oxynitrilase lysate, prepared as described in Example 3, 0.6 M trifluoroacetone, compound (1), 0.6 M nitromethane, compound (2), 0.1 M citrate buffer at pH 5.5. The reaction plates were heat-sealed and shaken at 150 rpm and 22° C.

After overnight incubation (˜22 hours), 300 μL/well of MTBE was added to the reaction plates. The quenched reaction plates were sealed, mixed well, and centrifuged at 4,000 rpm for 5 min. Subsequently a 150 μL aliquot of the top organic phase was removed from each well and added to a shallow well 96-well plate. For Selectivity determination the plates were sealed and analyzed by chiral GC using the analytical method described in Table 5-3. For the activity analysis by HPLC, 20 μL/well of the top organic phase of the quenched reaction plate was removed and added to a shallow well 96-well plate containing 180 μL of MeOH. The plates were sealed and mixed well. These samples were then analyzed by HPLC to determine the activity of the enzyme variants using the analytical method described in Table 5-1. Selected oxynitrilase variants showing a faster product formation of (IA), relative to SEQ ID NO:2 are shown in Table 6.1.

TABLE 6-1
Oxynitrilase Activity for the Production
of Compound (IA) Relative to SEQ ID NO: 2

| SEQ | | FIOP percent conversion Mean |
|---|---|---|
| ID NO: | Amino Acid Differences (Relative to | (T100% conv) (Relative to SEQ |
| (nt/aa) | SEQ ID NO: 2) | ID NO: 2)¹ |
| 3/4 | W39T/A105G/A261G | ++ |
| 5/6 | A105G/V146I/A261G | +++ |
| 7/8 | A105G | +++ |
| 9/10 | V143I | + |
| 11/12 | V12I/A105G/S113N | ++ |
| 13/14 | W39G/A105G/V202I/L260P | +++ |
| 15/16 | W39T | +++ |
| 17/18 | V12I/W39F/A105G/I110L/V202I/A261G | ++ |
| 19/20 | V12I/W39T/A261G | ++ |
| 21/22 | V12I/W39T/A105G/V202I/A261G | + |
| 23/24 | A99T | + |
| 25/26 | S98L | + |
| 27/28 | V12I/A105G/A261E | + |
| 29/30 | V12I/W39F/A261G | + |
| 31/32 | A105G/V202I | + |
| 33/34 | S98L/V202I/A261G | + |
| 35/36 | V12I/W39V/A105G | + |
| 37/38 | W39G/A105G/V146I | + |
| 39/40 | W39F/V97M | ++ |
| 41/42 | A261G | ++ |
| 43/44 | W39T/A261G | + |
| 45/46 | V12I/W39T/A105G | + |
| 47/48 | W39F/A105G | ++ |
| 49/50 | L260P/A261G | ++ |
| 51/52 | S211G | + |

¹Levels of increased activity were determined relative to the reference polypeptide of SEQ ID NO: 2 and defined as follows:
“+” 1.10 to 1.15 (first 50%),
“++” > 1.15 (next 30%),
“+++” > 1.22 (top 20%)

### Example 7

Round 8 Evolution and Screening of Engineered Polypeptides Derived from SEQ ID NO: 48 for Improved Production of Compound (IA)

The polynucleotide from example 6 SEQ TD NO: 47 encoding the most active polypeptide with oxynitrilase activity of SEQ TD NO: 48 was used to generate the engineered polypeptides of Table 7-1. These polypeptides displayed improved oxynitrilase activity under the desired conditions e.g., the improvement in the formation of the nitro alcohol compound (IA), that was produced in situ from the substrates trifluoroacetone and nitromethane, compounds (1) and (2), respectively, as compared to the starting polypeptide. Some polypeptides displayed improved product formation of nitro alcohol product (IA) compared to the starting polypeptide are noted in Table 7-1. The engineered polypeptides, having the amino acid sequences of even-numbered sequence identifiers were generated from the “backbone” amino acid sequence of SEQ ID NO: 48, as described below together with the analytical method described in Table 5-1.

Directed evolution began with the polynucleotide set forth in SEQ ID NO: 47. Libraries of engineered polypeptides were generated using various well-known techniques (e.g., saturation mutagenesis, recombination of previously identified beneficial amino acid differences) and screened using HTP assay and analysis methods, described below, that measured the polypeptides' ability to produce compound (IA).

The enzyme assays were carried out in 96-well deep-well (1.1 mL total volume) plates, in 100 μL total reaction volume per well. The reactions contained 20 v/v % of undiluted oxynitrilase lysate, prepared as described in Example 3, 0.62 M trifluoroacetone, compound (1), 0.63 M nitromethane, compound (2), citrate buffer at pH 5.5. The reaction plates were heat-sealed and shaken at 150 rpm and 22° C.

After overnight incubation (˜22 hours), 600 μL/well of MeOH was added to the reaction plates. The quenched reaction plates were sealed, mixed well, and centrifuged at 4,000 rpm for 15 min. Subsequently 20 μL/well of the quenched reaction plate was removed and added to a shallow well 96-well plate containing 180 μL of MeOH. The plates were sealed and mixed well. These samples were then analyzed by HPLC to determine the activity of the enzyme variants using the analytical method described in Table 5-1. Selected oxynitrilase variants showing a faster product formation of (IA), relative to SEQ ID NO:48 are shown in Table 7.1. The selectivity towards the (IA) product of the most active polypeptide SEQ ID NO: 74 was confirmed by chiral GC analysis using the analytical method described in Table 5-3 before continuing evolution as described in example 8. All variants showed an S-Selectivity >75% (>50% ee) under the given reaction conditions.

TABLE 7-1
Oxynitrilase Activity for the Production of
Compound (IA) Relative to SEQ ID NO: 48

| | | FIOP percent |
|---|---|---|
| | | conversion |
| | | Mean |
| | | (T100% conv) |
| SEQ | | (Relative to |
| ID NO: | Amino Acid Differences | SEQ ID NO: |
| (nt/aa) | (Relative to SEQ ID NO: 48) | 48)¹ |
| 53/54 | S122G | + |
| 55/56 | I9V/C13G/A261G | +++ |
| 57/58 | S122R/I132V/S211G/A261G | + |
| 59/60 | A99T/K175R/S211G/A261G | +++ |
| 61/62 | V146M/S211G/A261G | ++ |
| 63/64 | S122T | + |
| 65/66 | S122G/K175R/A261G | + |
| 67/68 | S211G/L260P | +++ |
| 69/70 | Q208M | + |
| 71/72 | N135Y | ++ |
| 73/74 | A154— | +++ |
| 75/76 | N243Q | + |
| 77/78 | N220T | + |
| 79/80 | R55C | ++ |
| 81/82 | H171A | ++ |
| 83/84 | E167M | + |
| 85/86 | D111G | + |
| 87/88 | L260Q | ++ |
| 89/90 | N160A | ++ |
| 91/92 | Q50E | + |
| 93/94 | Q208R | ++ |
| 95/96 | R55T | + |
| 97/98 | Q47S | +++ |
| 99/100 | R198M | ++ |
| 101/102 | L259R | + |
| 103/104 | Q247F | + |

¹Levels of increased activity were determined relative to the reference polypeptide of SEQ ID NO: 48 and defined as follows:
“+” 1.10 to 1.13 (first 50%),
“++” > 1.13 (next 30%),
“+++” > 1.17 (top 20%)

### Example 8

Round 9 Evolution and Screening of Engineered Polypeptides Derived from SEQ ID NO: 74 for Improved Production of Compound (IA) The polynucleotide from example 7 SEQ ID NO: 73 encoding the most active polypeptide with oxynitrilase activity of SEQ ID NO: 74 was used to generate the engineered polypeptides of Table 8-1. These polypeptides displayed improved oxynitrilase activity under the desired conditions e.g., the improvement in the formation of the nitro alcohol compound (IA), that was produced in situ from the substrates trifluoroacetone and nitromethane, compounds (1) and (2), respectively, as compared to the starting polypeptide. Some polypeptides displayed improved product formation of nitro alcohol product (IA) compared to the starting polypeptide are noted in Table 8-1. The engineered polypeptides, having the amino acid sequences of even-numbered sequence identifiers were generated from the “backbone” amino acid sequence of SEQ ID NO: 74, as described below together with the analytical method described in Table 8-1.

Directed evolution began with the polynucleotide set forth in SEQ ID NO: 73. Libraries of engineered polypeptides were generated using various well-known techniques (e.g., saturation mutagenesis, recombination of previously identified beneficial amino acid differences) and screened using HTP assay and analysis methods, described below, that measured the polypeptides' ability to produce compound (IA).

The enzyme assays were carried out in 96-well deep-well (1.1 mL total volume) plates, in 100 μL total reaction volume per well. The reactions contained 15 v/v % of undiluted oxynitrilase lysate, prepared as described in Example 3, 0.62 M trifluoroacetone, compound (1), 0.63 M nitromethane, compound (2), citrate buffer at pH 6.0. The reaction plates were heat-sealed and shaken at 150 rpm and 22° C.

After overnight incubation (˜22 hours), 300 μL/well of MeOH was added to the reaction plates. The quenched reaction plates were sealed, mixed well, and centrifuged at 4,000 rpm for 15 min. Subsequently 20 μL/well of the quenched reaction plate was removed and added to a shallow well 96-well plate containing 180 μL of MeOH. The plates were sealed and mixed well. These samples were then analyzed by HPLC to determine the activity of the enzyme variants using the analytical method described in Table 5-1. Selected oxynitrilase variants showing a faster product formation of (IA), relative to SEQ ID NO: 74 are shown in Table 8.1. Please note that oxynitrilase polypeptide with the SEQ ID NO: 142 was constructed rationally. It combines the beneficial mutations of polypeptide SEQ ID NO: 124 and SEQ ID NO: 140. The high selectivity towards the (IA) product of the most active polypeptides SEQ ID NO: 124, 140 and 142 was confirmed by chiral GC analysis using the analytical method described in Table 5-3 before continuing evolution as described in example 9. All variants showed an S-Selectivity >8000 (>6000 ee) under the given reaction conditions.

TABLE 8-1
Oxynitrilase Activity for the Production of
Compound (IA) Relative to SEQ ID NO: 74

| | | FIOP percent |
|---|---|---|
| | | conversion |
| | | Mean |
| SEQ | | (T100% conv) |
| ID NO: | Amino Acid Differences | (Relative to SEQ |
| (nt/aa) | (Relative to SEQ ID NO: 74) | ID NO: 74)¹ |
| 105/106 | V12I/R240K | ++ |
| 107/108 | V12I/L179E | + |
| 109/110 | V12I/R55E | ++ |
| 111/112 | V12I/V33A/R171L | + |
| 113/114 | V12I/S43I/N44D | + |
| 115/116 | V12I/R55E/R171L | + |
| 117/118 | V12I/V33A | ++ |
| 119/120 | V12I/R171L/L179E | + |
| 121/122 | V12I/R171L/R240K | ++ |
| 123/124 | V12I | +++ |
| 125/126 | V12I/R55E/S262A | ++ |
| 127/128 | Q207R/S231G | + |
| 129/130 | Q50E/R55C/S67R/S122G/Q207R | + |
| 131/132 | Q50E/T63S/S122G/H170A/N219T/S231G | + |
| 133/134 | S122G/Q207R/S231G | + |
| 135/136 | K129G | + |
| 137/138 | G81A | + |
| 139/140 | V2— | +++ |
| ²141/142 | V2—/V12I | n.d. |

¹Levels of increased activity were determined relative to the reference polypeptide of SEQ ID NO: 74 and defined as follows:
“+” 1.11 to 1.17 (first 50%),
“++” > 1.17 (next 30%),
“+++” > 1.28 (top 20%)
²SEQ ID 141/142 have been constructed rationally combining the best two mutations and haven't been part of the initial screening.
n.d.: not determined

### Example 9

Round 10 Evolution and Screening of Engineered Polypeptides Derived from SEQ ID NO: 142 for Improved Production of Compound (IA)

The polynucleotide from example 8 SEQ ID NO: 141 encoding the most active polypeptide with oxynitrilase activity of SEQ ID NO: 142 was used to generate the engineered polypeptides of Table 9-1. These polypeptides displayed improved oxynitrilase activity under the desired conditions e.g., the improvement in the formation of the nitro alcohol compound (IA), that was produced in situ from the substrates trifluoroacetone and nitromethane, compounds (1) and (2), respectively, as compared to the starting polypeptide. Some polypeptides displayed improved product formation of nitro alcohol product (IA) compared to the starting polypeptide are noted in Table 8-1. The engineered polypeptides, having the amino acid sequences of even-numbered sequence identifiers were generated from the “backbone” amino acid sequence of SEQ ID NO: 74, as described below together with the analytical method described in Table 8-1.

Directed evolution began with the polynucleotide set forth in SEQ ID NO:141. Libraries of engineered polypeptides were generated using various well-known techniques (e.g., saturation mutagenesis, recombination of previously identified beneficial amino acid differences) and screened using HTP assay and analysis methods, described below, that measured the polypeptides' ability to produce compound (IA).

The enzyme assays were carried out in 96-well deep-well (1.1 mL total volume) plates, in 100 μL total reaction volume per well. The reactions contained 10 v/v % of undiluted oxynitrilase lysate, prepared as described in Example 3, 0.6 M trifluoroacetone, compound (1), 0.9 M nitromethane, compound (2), citrate buffer at pH 6.0. The reaction plates were heat-sealed and shaken at 150 rpm and 22° C.

After overnight incubation (˜22 hours), 700 μL/well of MeOH was added to the reaction plates. The quenched reaction plates were sealed, mixed well, and centrifuged at 4,000 rpm for 15 min. Subsequently 20 μL/well of the quenched reaction plate was removed and added to a shallow well 96-well plate containing 180 μL of MeOH. The plates were sealed and mixed well. These samples were then diluted 1:400 with MeOH and analyzed by RF-MS to determine the activity of the enzyme variants using the analytical method described in Table 5-2. Selected oxynitrilase variants showing a faster product formation of (IA), relative to SEQ ID NO: 142 are shown in Table 9.1. The high selectivity towards the (IA) product of the most active polypeptides SEQ ID NO: 156 was confirmed by chiral GC analysis using the analytical method described in Table 5-3 before continuing evolution as described in example 10. All variants showed S-Selectivity >9000 (>80% ee) under the given reaction conditions.

TABLE 9-1
Oxynitrilase Activity for the Production of
Compound (IA) Relative to SEQ ID NO: 142

| | | FIOP |
|---|---|---|
| | | Product |
| | | Results Area |
| | | Mean |
| | | Relative |
| SEQ | | (Relative to |
| ID NO: | Amino Acid Differences | SEQ ID NO: |
| (nt/aa) | (Relative to SEQ ID NO: 142) | 142)¹ |
| 143/144 | I11V/Q49E/V123—/K128G/L257— | ++ |
| 145/146 | F38V/Q49E/I207V/R239K/L257— | ++ |
| 147/148 | I11V/F38V/Q49E/G80A/T151L/N154A/ | ++ |
| | N158M/I207V/S230G/R239K/L257— | |
| 149/150 | Q49E/L257— | + |
| 151/152 | Q49E/N154L/I207V/R239K/L257— | + |
| 153/154 | I11V/Q49E/T151L/N154A/N158C/Q206R/I207V/R239K | + |
| 155/156 | F38V/Q49E/T151L/N158M/Q206R/S230G | + |
| 157/158 | I207V/S230G | + |
| 159/160 | Q49E/T151L/N154A/N158C/I207V/R239K | + |
| 161/162 | Q49E/T151L/N154A/N158C/Q206R/I207V | + |
| 163/164 | D149Q/T151L/N158M/Q206R/S230G | + |
| 165/166 | F38V/S230G | + |
| 167/168 | E69N/G70K/K71G/L257— | + |
| 169/170 | L257— | + |
| 171/172 | L258* | +++ |
| 173/174 | S253—/A254—/S255—/D256—/L257—/L258—/A259—/V260— | ++ |
| 175/176 | D256* | +++ |
| 177/178 | L257N/A259R/V260* | ++ |
| 179/180 | N31T | + |
| 181/182 | L258A | + |
| 183/184 | A259T | ++ |
| 185/186 | Q206S | + |
| 187/188 | D110S | + |
| 189/190 | E146K | + |
| 191/192 | V260—/S261— | + |
| 193/194 | T143G | ++ |
| 195/196 | V260A | +++ |
| 197/198 | S189T | ++ |
| 199/200 | E190G | ++ |
| 201/202 | L257G | ++ |
| 203/204 | E190R | + |
| 205/206 | D126E | +++ |
| 207/208 | S255P | ++ |
| 209/210 | D256G | +++ |
| 211/212 | L257N | +++ |
| 213/214 | R196K | +++ |

¹Levels of increased activity were determined relative to the reference polypeptide of SEQ ID NO: 142 and defined as follows:
“+” 1.23 to 1.46 (first 50%),
“++” > 1.46 (next 30%),
“+++” > 1.69 (top 20%)

### Example 10

Round 11 Evolution and Screening of Engineered Polypeptides Derived from SEQ ID NO: 156 for Improved Stability in Isopropyl Acetate and Production of Compound (IA)

The polynucleotide from example 9 SEQ ID NO: 155 encoding the most active polypeptide with oxynitrilase activity of SEQ ID NO: 156 was used to generate the engineered polypeptides of Table 10-1. These polypeptides displayed improved oxynitrilase activity under the desired conditions e.g., the improvement in the formation of the nitro alcohol compound (IA), that was produced in situ from the substrates trifluoroacetone and nitromethane, compounds (1) and (2), respectively, as compared to the starting polypeptide. Some polypeptides displayed improved product formation of nitro alcohol product (IA) compared to the starting polypeptide are noted in Table 10-1. The engineered polypeptides, having the amino acid sequences of even-numbered sequence identifiers were generated from the “backbone” amino acid sequence of SEQ ID NO: 156, as described below together with the analytical method described in Table 8-3.

Directed evolution began with the polynucleotide set forth in SEQ ID NO: 155. Libraries of engineered polypeptides were generated using various well-known techniques (e.g., saturation mutagenesis, recombination of previously identified beneficial amino acid differences) and screened using HTP assay and analysis methods, described below, that measured the polypeptides' ability to produce compound (IA).

The enzyme assays were carried out in 96-well deep-well (1.1 mL total volume) plates, in 100 μL total reaction volume per well. The reactions contained 5 v/v % of undiluted oxynitrilase lysate, prepared as described in Example 3, 0.6 M trifluoroacetone, compound (1), 0.9 M nitromethane, compound (2), 30% isopropylacetate in citrate buffer at pH 6.0. The reaction plates were heat-sealed and shaken at 150 rpm and 22° C.

After overnight incubation (˜22 hours), 700 μL/well of MeOH was added to the reaction plates. The quenched reaction plates were sealed, mixed well, and centrifuged at 4,000 rpm for 15 min.

Subsequently 20 μL/well of the quenched reaction plate was removed and added to a shallow well 96-well plate containing 180 μL of MeOH. The plates were sealed and mixed well. These samples were then diluted 1:400 with MeOH and analyzed by RF-MS to determine the activity of the enzyme variants using the analytical method described in Table 5-2. Selected oxynitrilase variants showing a faster product formation of (IA), relative to SEQ ID NO: 156 are shown in Table 10.1. The high selectivity towards the (IA) product of the most active polypeptides SEQ ID NO: 234 was confirmed by chiral GC analysis using the analytical method described in Table 5-3 before continuing evolution as described in example 11. All variants showed a S-Selectivity >92% (>84% ee) under the given reaction conditions.

TABLE 10-1
Oxynitrilase Activity for the Production of
Compound (IA) Relative to SEQ ID NO: 156

| | | FIOP |
|---|---|---|
| | | Product |
| | | Results Area |
| | | Mean |
| SEQ | | (Relative to |
| ID NO: | Amino Acid Differences | SEQ ID |
| (nt/aa) | (Relative to SEQ ID NO: 156) | NO: 156)¹ |
| 215/216 | N31T/E49D/R54G/I131G/E146K/I207V/Q248G | + |
| 217/218 | N31T/E49D/R54G/E146K/S194G/R206Q/I207V/L258* | + |
| 219/220 | N31T/R54G/E146K/M158N/T183R/E190R/R206Q/I207V/ | + |
| | Q248G/L258* | |
| 221/222 | N31T/T183R | ++ |
| 223/224 | E49D/R54G/E146K/T183R/E190R/S194G/R206S/I207V/Q248G | ++ |
| 225/226 | N31T/L151T/I207V/Q248G | ++ |
| 227/228 | N31T | ++ |
| 229/230 | E146K/L151T/M158N/R206Q/I207V | ++ |
| 231/232 | R54G/L151T/R206Q/I207V/D256E | ++ |
| 233/234 | N31T/D110S/L151T/R206S | +++ |
| 235/236 | E146K/L151T/R206S/I207V/Q248G | ++ |
| 237/238 | N31T/E49D/R54G/I131G/E146K/L151T/T183R | + |
| 239/240 | N31T/E49D/R54G/D110S/E146K/T183R/S194G/R206Q/I207V/ | +++ |
| | Q248G/D256* | |
| 241/242 | N31T/S194G/R206Q | ++ |
| 243/244 | E49D/R54G/L151T/S194G/I207V | +++ |
| 245/246 | N31T/R206Q/I207V | ++ |
| 247/248 | D110S/L151T/D182Y/T183R/R206S/I207V | + |
| 249/250 | N31T/E49D/T183R/I207V | + |
| 251/252 | R54G/I131G/L151T/S194G/Q248G | ++ |
| 253/254 | N31T/R54G/E146K/E190R/R206S/I207V | + |
| 255/256 | R54G/I207V/Q248G | + |
| 257/258 | N31T/R54G/I131G/E146K/L151T/R206Q/Q248G | + |
| 259/260 | E49D/R54G/R206Q/I207V | + |
| 261/262 | N31T/R54G/D110S/E146K/L151T/M158N/T183R/R206S/I207V | ++ |
| 263/264 | N31T/E146K | ++ |
| 265/266 | S194G/R206S/I207V | + |
| 267/268 | N31T/E146K/E190R/S194G/R206S/I207V | +++ |
| 269/270 | N31T/I131G/L151T/I207V | ++ |
| 271/272 | N31T/E49D/R54G/E146K/L151T/T183R/E190R | + |
| 273/274 | E49D/R54G/L151T/R206Q/I207V | +++ |
| 275/276 | N31T/R206S | +++ |
| 277/278 | N31T/R54G/E146K/L151T/S194G/R206Q | +++ |
| 279/280 | E49D/R54G/E146K/L151T/R206Q/S255P/L257— | + |
| 281/282 | N31T/E49D/T183R/R206Q/I207V | + |
| 283/284 | N31T/R54G/E146K/S194G/R206Q/I207V | + |
| 285/286 | N31T/L151T/E190R/R206S/Q248G | + |
| 287/288 | E49D/T183R/E190R/R206Q/I207V/Q248G | + |
| 289/290 | E146K/L151T/R206Q/I207V/Q248G | + |
| 291/292 | E49D/R54G/E146K/R206S/I207V/Q248G | + |
| 293/294 | N31T/E49D/E146K/L151T/I207V/S255* | ++ |
| 295/296 | N31T/R54G/E190R/S194G/R206S/Q248G/D256* | + |
| 297/298 | N31T/M158N/R206Q/Q248G | + |
| 299/300 | C12G | +++ |
| 301/302 | V116Q | + |
| 303/304 | L7F | + |
| 305/306 | A114G | ++ |
| 307/308 | L151A | + |
| 309/310 | A244S | +++ |
| 311/312 | D108S | + |

¹Levels of increased activity were determined relative to the reference polypeptide of SEQ ID NO: 156 and defined as follows:
“+” 1.29 to 1.46 (first 50%),
“++” > 1.46 (next 30%),
“+++” > 1.55 (top 20%)

### Example 11

Round 12 Evolution and Screening of Engineered Polypeptides Derived from SEQ ID NO: 234 for Improved Production of Compound (IA)

The polynucleotide from example 10 SEQ ID NO: 233 encoding the most active polypeptide with oxynitrilase activity of SEQ ID NO: 234 was used to generate the engineered polypeptides of Table 11-1.

These polypeptides displayed improved oxynitrilase activity under the desired conditions e.g., the improvement in the formation of the nitro alcohol compound (IA), that was produced in situ from the substrates trifluoroacetone and nitromethane, compounds (1) and (2), respectively, as compared to the starting polypeptide. Some polypeptides displayed improved product formation of nitro alcohol product (IA) compared to the starting polypeptide are noted in Table 11-1. The engineered polypeptides, having the amino acid sequences of even-numbered sequence identifiers were generated from the “backbone” amino acid sequence of SEQ ID NO: 234, as described below together with the analytical method described in Table 8-3.

Directed evolution began with the polynucleotide set forth in SEQ ID NO: 233. Libraries of engineered polypeptides were generated using various well-known techniques (e.g., saturation mutagenesis, recombination of previously identified beneficial amino acid differences) and screened using HTP assay and analysis methods, described below, that measured the polypeptides' ability to produce compound (IA).

The enzyme assays were carried out in 96-well deep-well (1.1 mL total volume) plates, in 100 μL total reaction volume per well. The reactions contained 2.5 v/v % of undiluted oxynitrilase lysate, prepared as described in Example 3, 0.75 M trifluoroacetone, compound (1), 0.9 M nitromethane, compound (2), 30% isopropyl acetate in citrate buffer at pH 6.0. The reaction plates were heat-sealed and shaken at 150 rpm and 22° C.

After overnight incubation (˜22 hours), 650 μL/well of MeOH was added to the reaction plates. The quenched reaction plates were sealed, mixed well, and centrifuged at 4,000 rpm for 15 min.

Subsequently 15 μL/well of the quenched reaction plate was removed and added to a shallow well 96-well plate containing 185 μL of MeOH. The plates were sealed and mixed well. These samples were then diluted 1:400 with MeOH and analyzed by RF-MS to determine the activity of the enzyme variants using the analytical method described in Table 5-2. Selected oxynitrilase variants showing a faster product formation of (IA), relative to SEQ ID NO: 234 are shown in Table 11.1. The high selectivity towards the (IA) product of the most active polypeptides SEQ ID NO: 410 was confirmed by chiral GC analysis using the analytical method described in Table 5-3 before continuing evolution as described in example 12. All variants showed a S-Selectivity >9200 (>8400 ee) under the given reaction conditions.

TABLE 11-1
Oxynitrilase Activity for the Production of
Compound (IA) Relative to SEQ ID NO: 234

| | | FIOP |
|---|---|---|
| | | Product |
| | | Results |
| | | Area |
| SEQ | | (Relative |
| ID NO: | Amino Acid Differences | to SEQ ID |
| (nt/aa) | (Relative to SEQ ID NO: 234) | NO: 234)¹ |
| 313/314 | S40C/V200I/S206Q/A231G/I235P | + |
| 315/316 | A98G/S206Q/I207V | + |
| 317/318 | S206Q/I207V | + |
| 319/320 | T31N/S110D/T151L/S206R | ++ |
| 321/322 | T151S | + |
| 323/324 | L47M/S206R/I207V | ++ |
| 325/326 | T151S/S206Q/I207T | +++ |
| 327/328 | S110D | ++ |
| 329/330 | T151L | ++ |
| 331/332 | I22G | +++ |
| 333/334 | I22G/S206Q/I207C/A244S | +++ |
| 335/336 | I22G/S206Q/I207C | + |
| 337/338 | L17I | ++ |
| 339/340 | S206Q/I207T | ++ |
| 341/342 | I207V | + |
| 343/344 | L184M/I217V | +++ |
| 345/346 | L63A | ++ |
| 347/348 | S27G | ++ |
| 349/350 | Y201C | ++ |
| 351/352 | W127A | +++ |
| 353/354 | L63E | + |
| 355/356 | F187L | + |
| 357/358 | A254G | ++ |
| 359/360 | V38Y | + |
| 361/362 | A28W | +++ |
| 363/364 | F124T | + |
| 365/366 | Y226L | ++ |
| 367/368 | W127S | ++ |
| 369/370 | S112G | +++ |
| 371/372 | I195S | ++ |
| 373/374 | V38C | + |
| 375/376 | Y201L | + |
| 377/378 | L184Q | ++ |
| 379/380 | Y201E | + |
| 381/382 | L152I | + |
| 383/384 | H169R | +++ |
| 385/386 | V164L | +++ |
| 387/388 | L63Q | +++ |
| 389/390 | F124D | + |
| 391/392 | Y201S | ++ |
| 393/394 | E49D/R54G/E146K/T151L/T183R/S194G/S206Q/I207V/Q248G | + |
| 395/396 | S40C/E49D/R54G/E146K/T151L/T183R/S194G/S206Q/I207V/ | + |
| | Q248G/D256* | |
| 397/398 | E49D/R54G/A98G/E146K/T151L/T183R/S194G/S206Q/I207V/ | + |
| | Q248G | |
| 399/400 | R54G/E146K/T151L/T183R/S194G/S206Q/I207V/Q248G/D256* | + |
| 401/402 | E49D/R54G/V116Q/E146K/T151L/T183R/S194G/S206Q/I207V/ | + |
| | A231G/Q248G/D256* | |
| 403/404 | F5V/R54G/E146K/T151L/T183R/S194G/S206Q/I207V/Q248G/ | + |
| | D256* | |
| 405/406 | E49D/R54G/T151L/T183R/S194G/Y199G/S206Q/I207V/A231G/ | + |
| | Q248G/D256* | |
| 407/408 | E49D/R54G/E146K/T151L/T183R/S194G/S206Q/I207V/A231G/ | + |
| | Q248G/D256* | |
| 409/410 | R54G/E146K/T151L/T183R/S194G/S206Q/I207V/A231G/Q248G/ | + |
| | D256* | |
| 411/412 | F5V/E49D/R54G/E146K/T151L/T183R/S194G/S206Q/I207V/ | + |
| | Q248G/D256* | |

¹Levels of increased activity were determined relative to the reference polypeptide of SEQ ID NO: 234 and defined as follows:
“+” 1.18 to 1.29 (first 50%),
“++” > 1.29 (next 30%),
“+++” > 1.41 (top 20%)

### Example 12

Round 13 Evolution and Screening of Engineered Polypeptides Derived from SEQ ID NO: 410 for Improved Production of Compound (IA)

The polynucleotide from example 11 SEQ ID NO: 409 encoding the most active polypeptide with oxynitrilase activity of SEQ ID NO: 410 was used to generate the engineered polypeptides of Table 12-1. These polypeptides displayed improved oxynitrilase activity under the desired conditions e.g., the improvement in the formation of the nitro alcohol compound (IA), that was produced in situ from the substrates trifluoroacetone and nitromethane, compounds (1) and (2), respectively, as compared to the starting polypeptide. Some polypeptides displayed improved product formation of nitro alcohol product (IA) compared to the starting polypeptide are noted in Table 12-1. The engineered polypeptides, having the amino acid sequences of even-numbered sequence identifiers were generated from the “backbone” amino acid sequence of SEQ ID NO: 410, as described below together with the analytical method described in Table 8-3.

Directed evolution began with the polynucleotide set forth in SEQ ID NO: 409. Libraries of engineered polypeptides were generated using various well-known techniques (e.g., saturation mutagenesis, recombination of previously identified beneficial amino acid differences) and screened using HTP assay and analysis methods, described below, that measured the polypeptides' ability to produce compound (IA).

The enzyme assays were carried out in 96-well deep-well (1.1 mL total volume) plates, in 100 μL total reaction volume per well. The reactions contained 5 v/v % of undiluted oxynitrilase lysate, prepared as described in Example 3, 0.75 M trifluoroacetone, compound (1), 0.9 M nitromethane, compound (2), 30% isopropyl acetate in citrate buffer at pH 6.0. The reaction plates were heat-sealed and shaken at 150 rpm and 22° C.

After overnight incubation (˜22 hours), 650 μL/well of MeOH was added to the reaction plates. The quenched reaction plates were sealed, mixed well, and centrifuged at 4,000 rpm for 15 min.

Subsequently 15 μL/well of the quenched reaction plate was removed and added to a shallow well 96-well plate containing 185 μL of MeOH. The plates were sealed and mixed well. These samples were then analyzed by HPLC to determine the activity of the enzyme variants using the analytical method described in Table 5-1. Selected oxynitrilase variants showing a faster product formation of (IA), relative to SEQ ID NO: 410 are shown in Table 12.1. The high S-selectivity of >95% (>90% ee) towards the (IA) product of the most active polypeptides SEQ ID NO: 414 was confirmed by chiral GC analysis using the analytical method described in Table 5-3 before continuing evolution as described in example 13. All other variants also showed a high S-selectivity >94% (>88% ee) under the given reaction conditions.

TABLE 12-1
Oxynitrilase Activity for the Production of
Compound (IA) Relative to SEQ ID NO: 410

| | | FIOP percent |
|---|---|---|
| | | conversion |
| SEQ | | Mean (Relative |
| ID NO: | Amino Acid Differences | to SEQ ID NO: |
| (nt/aa) | (Relative to SEQ ID NO: 410) | 410)¹ |
| 413/414 | S27G/A28W/L63A/G194S/Y201C | +++ |
| 415/416 | L17I/L63A/S112G/Y226L | +++ |
| 417/418 | L17I/I22G/L63Q/S112G/K146E/G248Q | ++ |
| 419/420 | I22R/S27G/A28W/H169R/G194S/I195S | ++ |
| 421/422 | I22G/H169R/G194S/I195S | + |
| 423/424 | L17I/L63A/S112G/Y201C/Y226L | +++ |
| 425/426 | L17I/L63Q/S112G | ++ |
| 427/428 | L17I/S27G/L63Q/K146E/H169R/Y201C | +++ |
| 429/430 | L17I/S27G/A28W/L63A/S112G/I195S/G248Q | ++ |
| 431/432 | I22G/S112G/L151T/M158N/H169R/V207T | +++ |
| 433/434 | V38F/S110D/W127A/K146E/L151T/V207T | ++ |
| 435/436 | D182Q | + |
| 437/438 | S255M | ++ |
| 439/440 | K119G | + |
| 441/442 | T143V | + |
| 443/444 | N154G | + |
| 445/446 | N134F | ++ |
| 447/448 | D149T | + |
| 449/450 | V123T | +++ |
| 451/452 | D182W | ++ |
| 453/454 | N154S | +++ |
| 455/456 | D149V | +++ |
| 457/458 | K128S | ++ |
| 459/460 | S255N | ++ |
| 461/462 | N252K | +++ |
| 463/464 | A3M | ++ |
| 465/466 | D149H | ++ |
| 467/468 | N154V | +++ |
| 469/470 | P107V | ++ |
| 471/472 | S255K | ++ |
| 473/474 | I8V | + |
| 475/476 | D149S | +++ |
| 477/478 | L17V | ++ |
| 479/480 | N134W | ++ |
| 481/482 | D126M | ++ |
| 483/484 | K118N/P229S | +++ |
| 485/486 | A254L | +++ |
| 487/488 | N134C | +++ |
| 489/490 | I8C | + |
| 491/492 | D182L | +++ |
| 493/494 | Q213F | +++ |
| 495/496 | M106Q | ++ |
| 497/498 | D180S | ++ |
| 499/500 | A254V | + |
| 501/502 | V102L | + |
| 503/504 | K146A | + |
| 505/506 | V117A | ++ |
| 507/508 | S253V | +++ |
| 509/510 | K119S | + |
| 511/512 | D182A | + |
| 513/514 | Q216E | + |
| 515/516 | E122A | + |
| 517/518 | S253I | + |
| 519/520 | K119V | + |
| 521/522 | E122S | + |
| 523/524 | S209L | + |
| 525/526 | I83V | + |
| 527/528 | A79G | + |
| 529/530 | I8L | + |
| 531/532 | S133C | + |
| 533/534 | S255R | + |
| 535/536 | T141V | + |
| 537/538 | S2L | + |
| 539/540 | S2T | ++ |
| 541/542 | Q179S | + |
| 543/544 | E122G | + |
| 545/546 | K173C | + |
| 547/548 | D182V | + |
| 549/550 | Q206L | + |
| 551/552 | Q213V | + |
| 553/554 | L151V | + |
| 555/556 | E122M | + |
| 557/558 | K173W | + |
| 559/560 | S255W | + |
| 561/562 | N154A | + |
| 563/564 | M106L | ++ |
| 565/566 | I83M | + |
| 567/568 | T141Q | + |
| 569/570 | E122T | + |
| 571/572 | I131G | + |
| 573/574 | S255V | ++ |
| 575/576 | D182G | + |
| 577/578 | K173L | +++ |
| 579/580 | Q213M | +++ |
| 581/582 | N134V | ++ |
| 583/584 | S255Q | + |
| 585/586 | L178I | + |
| 587/588 | F101L | + |
| 589/590 | K119R | ++ |
| 591/592 | S253C | ++ |
| 593/594 | S2G | ++ |
| 595/596 | K118R | ++ |
| 597/598 | K128M | + |
| 599/600 | D149G | ++ |
| 601/602 | S121V | + |
| 603/604 | K128R | + |

¹Levels of increased activity were determined relative to the reference polypeptide of SEQ ID NO: 410 and defined as follows:
“+” 1.23 to 1.74 (first 50%),
“++” > 1.74 (next 30%),
“+++” > 2.12 (top 20%)

### Example 13

### Comparison of the Catalytic Activity and Selectivity of the Wildtype Polypeptide SEQ ID NO: 606 and the Engineered Polypeptides SEQ ID NO: 2

Both, the polynucleotides SEQ ID NO: 605 encoding for the wildtype (S)-hydroxynitrile lyase from Baliospermum montanum, Uniprot ID: D1MX73 with the SEQ ID NO: 606 and the engineered polynucleotide SEQ ID NO: 1 encoding for the starting enzyme from example 6 with the SEQ ID NO: 2, have been used for SFP production as described in example 4.

The catalytic activity and selectivity to convert the substrates trifluoroacetone (1) and nitromethane (2) to the desired nitro alcohol compound (IA) was conducted in a 96-well deep well plate (1.1 mL total volume) with 100 μL total reaction volume per well. The reactions contained 0 g/L, 0.049 g/L, 0.098 g/L, 0.195 g/L, 0.391 g/L, 0.781 g/L, 1.563 g/L, 3.125 g/L, 6.25 g/L, 12.5 g/L, 25 g/L, 50 g/L of SFP, prepared as described in Example 3, 0.623 M (70 g/L) trifluoroacetone (1), 0.631 M (39 g/L) nitromethane (2), in 100 mM citrate buffer at pH 5.5. Each reaction was conducted in duplicate. Two reaction plates were set-up in parallel, one was used for chiral GC analysis, the other one for achiral LC analysis. Both reaction plates were heat-sealed and shaken at 150 rpm and 22° C. After overnight incubation (˜22 hours), 450 μL/well of MTBE was added to the GC reaction plate and 650 μL/well of MeOH was added to the LC reaction plate. The quenched reaction plates were sealed, mixed well, and centrifuged at 4,000 rpm for 15 min.

Subsequently 15 μL/well of the quenched LC reaction plate was removed and added to a shallow well 96-well plate containing 185 μL of MeOH. The plate was sealed and mixed well. These samples were then analyzed by HPLC to determine the activity of the enzyme variants using the analytical method described in Table 5-1.

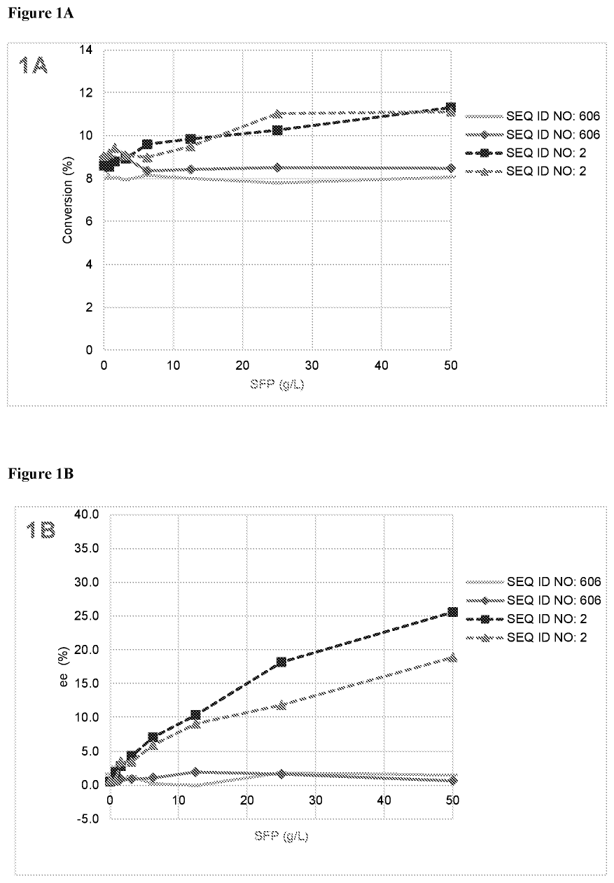

For the selectivity and enantiomeric excess (ee) analysis 150 μL/well of the top organic phase of the quenched GC reaction plate was removed and added to a shallow well 96-well plate. This plate was then analyzed by chiral GC utilizing the analytical method described in Table 5-3. FIG. 1 clearly shows the higher selectivity and activity of SEQ ID 2 compared to the wildtype polypeptide SEQ ID 606.

Comparative data in FIGS. 1A and 1B show the percentage conversion and enantiomeric excess, respectively, of trifluoroacetone (1) and nitromethane (2) to the nitro alcohol compound (IA) with the wildtype (S)-hydroxynitrile lyase from Baliospermum montanum, Uniprot ID: D1MX73 having SEQ ID NO: 606 versus the engineered polypeptide SEQ ID NO: 2. The experiment was conducted in duplicate. The reaction mixture contained 0.623 M (70 g/L) trifluoroacetone (1), 0.631 M (39 g/L) nitromethane (2), in 100 mM citrate buffer at pH 5.5 and varying SFP catalyst concentrations from 0-50 g/L. The engineered polypeptides with the SEQ ID NO: 2 showed a higher selectivity and activity than the wildtype polypeptide SEQ ID NO: 606. As the selective enzymatic reaction is always in competition with the unselective chemical reaction in parallel, the selectivity of the SEQ ID NO: 606 oxynitrilase (>90%) only resulted in an actual ee of max 25%. Therefore the evolution campaign described in this disclosure was set out to further improve the activity of the enzyme while maintaining the high selectivity.

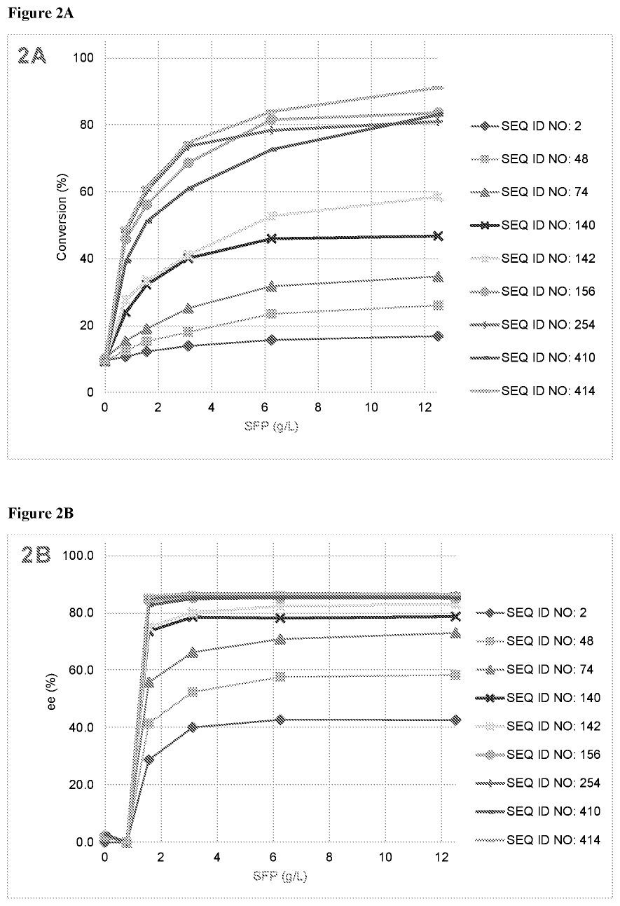

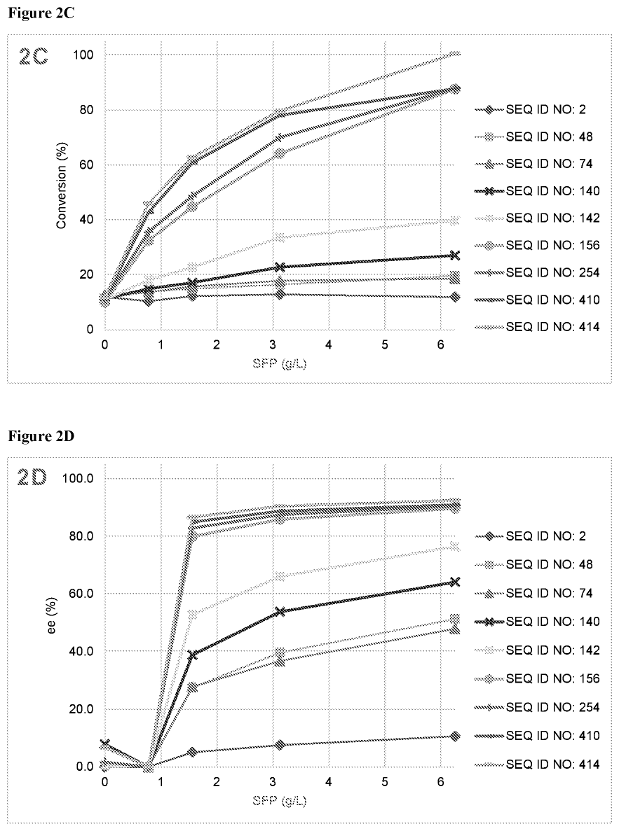

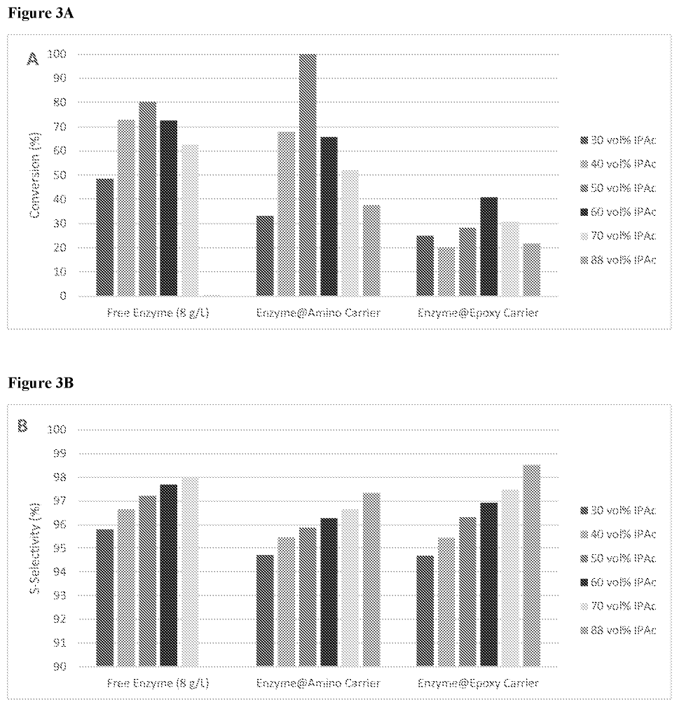

In a follow up experiment the best enzyme of each evolution round has been used for the transformation of the substrates trifluoroacetone (1) and nitromethane (2) to the desired nitro alcohol compound (IA). If not stated otherwise the experimental procedure is the same as in FIGS. 1A and 1B showing the comparison to the wildtype sequence. The enzyme SFPs have been prepared as described in example 3 and used in the following concentrations 0 g/L, 0.049 g/L, 0.098 g/L, 0.195 g/L, 0.391 g/L, 0.781 g/L, 1.563 g/L, 3.125 g/L, 6.25 g/L, 12.5 g/L SFP. Two different conditions have been investigated, (1^{st}): Initial screening conditions of example 6 and 7 with 0.623 M (70 g/L) trifluoroacetone (1), 0.631 M (39 g/L) nitromethane (2), in 100 mM citrate buffer at pH 5.5 (see FIG. 2A, 2B) and (2^{nd}): Final screening conditions of example 11, 12 and 13 with 0.75 M (84 g/L) trifluoroacetone (1), 0.9 M (55 g/L) nitromethane (2), 30% isopropylacetate in 100 mM citrate buffer at pH 6.0 (see FIG. 2C, 2D). FIGS. 2A and 2C show the percentage conversion whereas FIGS. 2B and 2D show the enantiomeric excess of the most active polypeptides from each round for the conversion of trifluoroacetone (1) and nitromethane (2) to the nitro alcohol compound (IA). As shown in FIG. 2 the ranking of the different enzyme variants is matching well under both conditions to the progress made from round to round. The oxynitrilase with the SEQ ID NO: 414 shows the highest selectivity and conversion under both conditions. However while under 1^{st} condition SEQ ID NO: 414 with 12.5 g/L SFP loading reached 91% conversion, and 87% ee (see FIGS. 2A, 2B), it reached with an loading of only 6.25 g/L under the improved 2^{nd} reaction conditions 100% conversion and 93% ee (see FIGS. 2C, 2D). The enzyme having SEQ ID NO: 414 was then used in a scale-up reaction, see example 14.

### Example 14

Production of Nitro Alcohol Compound (IA), from the Substrates Trifluoroacetone (1) and Nitromethane (2) with Engineered Polypeptide SEQ ID NO: 414 as Catalyst

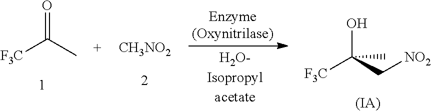

The most active polypeptide with oxynitrilase activity from example 12, SEQ ID NO: 414 was produced as fermentation powder and used to convert the substrates trifluoroacetone (1) and nitromethane (2) to the nitro alcohol compound (IA). 0.504 g of polypeptide SEQ ID NO: 414 was dissolved in sodium citrate buffer (4.0 mL, 0.1 M, pH 6.0±0.5) and loaded into a reactor pre-cooled to 10° C. A 10° C. solution of trifluoroacetone (12.13 g, 108.3 mmol) in sodium citrate buffer (27.8 mL, 0.1 M, pH 6.0), and additional sodium citrate buffer (78.6 mL, 0.1 M, pH 6.0) were charged to the reactor. The internal temperature and stirring was set to 22° C. and 350 rpm before nitromethane (13.2 g, 11.7 mL, 216.5 mmol), followed by isopropyl acetate (10.2 g, 11.7 mL), was added. Additional isopropyl acetate (21.1 g, 24.3 mL) was added and stirred at 22° C. for 22 h. CellFlock 40 (6.0 g) and isopropyl acetate (5.0 g, 5.7 mL) were added to the emulsion and stirred at 22° C. for 2 h. The resulting suspension was filtered to afford a clear biphasic mixture. The aqueous layer was separated and extracted with isopropyl acetate. The combined organic layers were concentrated at 50° C. jacket temperature and 180-200 mbar. Some isopropyl acetate was added to the obtained distillation residue, that was again concentrated. This sequence was repeated one more time to obtain the nitro alcohol at the desired concentration in isopropyl acetate (91.7 g solution containing 16.84 g nitro alcohol, 90.8% yield, e.r.=96:4). ¹H NMR (DMSO-d6): 6.96 (s, OH), 4.92 (d, 1H), 4.82 (d, 1H), 1.48 (s, 3H)

### Example 15

Production of Nitro Alcohol Compound (IA), from the Substrates Trifluoroacetone (1) and Nitromethane (2) with Whole Cells Contained the Engineered Polypeptide SEQ ID NO: 254

The polynucleotide from Example 10 having SEQ ID NO: 233 encoding the most active polypeptide from Example 10 with oxynitrilase activity having SEQ ID NO: 234 was produced in the three different enzyme formulation forms as described in Example 4, e.g., as SFP, FWC or LWC. All the three formulations allowed successful formation of the nitro alcohol compound (IA), that was produced in situ from the substrates trifluoroacetone and nitromethane, compounds (1) and (2), respectively.

The enzyme assays were carried out in 96-well deep-well (1.1 mL total volume) plates, in 100 μL total reaction volume per well. The reactions contained one of the three oxynitrilase formulations (SFP, LWC or FWC) and 0.62 M trifluoroacetone, compound (1), 0.98 M nitromethane, compound (2), 30% isopropyl acetate in citrate buffer at pH 6.0. The reaction plates were heat-sealed and shaken at 150 rpm and 22° C.
After overnight incubation (˜22 hours), 300 μL/well of MTBE was added to the reaction plates. The quenched reaction plates were sealed, mixed well, and centrifuged at 4,000 rpm for 5 min. Subsequently a 150 μL aliquot of the top organic phase was removed from each well and added to a shallow well 96-well plate. For Selectivity determination the plates were sealed and analyzed by chiral GC using the analytical method described in Table 5-3. For the activity analysis by HPLC, 20 μL/well of the top organic phase of the quenched reaction plate was removed and added to a shallow well 96-well plate containing 180 μL of MeOH. The plates were sealed and mixed well. These samples were then analyzed by HPLC to determine the activity of the enzyme variants using the analytical method described in Table 5-1.

Using the catalyst in the three formulations: SFP, FWC or LWC originating from the same amounts of cells normalized to a loading of 20 μg/l SFP, 50% conversion to (IA) was observed utilizing the SFP formulation, 55% conversion for the LWC formulation and 65% conversion using FWCs after 20 hours reaction time. All samples showed an S-Selectivity of 93%.

### Example 16

### Immobilization of Engineered Polypeptide SEQ ID NO: 410

Fermentation powder of the polypeptide SEQ ID NO: 410 with oxynitrilase activity was immobilized on solid methoxy methacrylate carriers. This allows faster filtration and recycling of the polypeptide thus enable more flexible process options for nitroaldol reaction from substrates (1) and (2) to product (IA). As a proof on concept polypeptide SEQ ID NO: 410 was crosslinked on epoxy (ECR8204F) or amino-functionalized methacrylate resins (ECR8304F) from Purolite Inc. For this, 6 g of each resin was used and all washing steps were performed in the ratio of 1:4 (resin:washing solution). The resins were initially washed with immobilization buffer, 1 M (for ECR8204F) and 50 mM (for ECR8304F)) sodium citrate buffer pH 6.0. For the crosslinking on amino-functionalized methacrylate resins (ECR8304F) incubation with 1% glutardialdehyde solution for 2 hours was conducted and the activated resin was washed three times with immobilization buffer. Afterwards both resins were incubated with at 15 mg/mL solution of lyophilized polypeptide SEQ ID: 410 in immobilization buffer overnight. Both types of resin (ECR8304F & ECR8204F) were washed once with 20 mM sodium citrate buffer pH 6.0 and twice with 0.5 M brine. Afterwards both resins were washed with 50 mM sodium citrate buffer pH 6.0 three times.

These resins were then used under similar reaction conditions as in example 12, 170 mg immobilized enzyme was incubated for 22 h, 22° C. in 1 ml 0.1 M citrate buffer pH 6 containing 0.85 M (1), 1.02 M (2) and isopropyl acetate (IPAc) concentrations between 20% to 88%. After overnight incubation, 650 μL/well of MeOH was added to the reaction plates. The quenched reaction plates were sealed, mixed well, and centrifuged at 4,000 rpm for 15 min. Subsequently μL/well of the quenched reaction plate was removed and added to a shallow well 96-well plate containing 185 μL of MeOH. The plates were sealed and mixed well. These samples were then analyzed by HPLC to determine the activity of the enzyme variants using the analytical method described in Table 5-1. FIG. 3 shows the Activity (See FIG. 3A) and Selectivity (See FIG. 3B) of the free enzyme compared to the immobilized enzymes on either amino or epoxy carrier. As shown in FIG. 3 the amino resin outperformed the epoxy resin in terms of conversion (See FIG. 3A) and immobilization yield, while selectivity was similar in all cases (See FIG. 3B). High isopropyl acetate (IPAc) concentrations (ranging between 20% and 88%) were applied in the screening. Please Note that the substrate solution itself had a volume of 12% and for samples with 88 vol % IPAc the IPAc was water-saturated prior to the reaction.

The conditions for the amino resin had been further optimized and the best conditions have been used for an upscaled immobilization reaction. For this, 30 g of ECR8304F from Purolite Inc. was used and all washing steps were performed in the ratio of 1:4 (resin:washing solution). The resins were initially washed with 50 mM sodium citrate pH 6.0 immobilization buffer. For the crosslinking incubation with 1% glutardialdehyde solution for 2 hours was conducted and the activated resin was washed three times with immobilization buffer. Afterwards both resins were incubated with a 20 mg/mL solution of FP of the polypeptide SEQ ID: 410 in immobilization buffer overnight. Both resins were washed once with 20 mM sodium citrate buffer pH 6.0, twice with 0.5 M brine and twice with buffer. This procedure resulted in a immobilization yield of 70-80% and an overall recovered enzyme activity of ˜30%. The so generated 30 g of immobilized polypeptide SEQ ID NO: 410 was incubated for 22 h, 22° C. in 180 ml 0.1 M citrate buffer pH 6 containing 0.61 M (1), 1.22 M (2) and 30% IPAc resulting in 80% yield and 92% ee (96% S-Selectivity). This immobilized enzyme was recycled two times by filtering the reaction mixture and no significant loss in reaction yield and selectivity was observed. The second recycling was done after 25 days of total storage time at 4° C.

### Example 17

### Hydrogenation of Compound of Formula (IA)

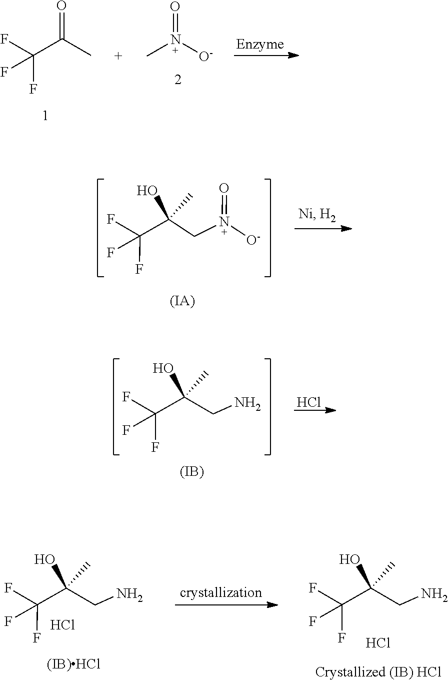

### Batch Procedure

(IA) in isopropyl acetate (70.0 g, 13.7 wt.-%, 9.6 g pure (IA)) was placed in a pressure vessel. Water wet Nickel sponge catalyst (Raney Ni) (0.96 g, 10 wt.-% to pure (IA)) was added, followed by methanol (20 g, 15.8 mL). The vessel was made inert with Nitrogen and the atmosphere was then changed to hydrogen. The resulting suspension was stirred for 6 h at 40° C. under a positive pressure of hydrogen (20 bar). The reaction mixture was then cooled to room temperature, filtered in a manner to keep the filter residue wet to avoid ignition on contact with air. The conversion to (IB) was measured using GC (99% conversion).

HCl (1.25 M in MeOH, 2.0 eq) was added and stirred at room temperature for 6 h. The resulting solution was filtered before being concentrated at 40° C. jacket temperature and 60 mbar. Some isopropyl acetate was added to the obtained distillation residue, that was again concentrated. This sequence was repeated two more times to obtain the HCl salt of the amino alcohol free of methanol. Acetonitrile was added and the obtained solution was heated to 35° C. jacket temperature before n-heptane followed by a suspension of product seed material in n-heptane (one part (IB). HCl and 33 parts n-heptane) was added slowly. Additional n-heptane was added and the resulting mixture was slowly cooled to 5° C. jacket temperature. Stirring was continued for 18 h. The suspension was filtered, and the filter cake was washed with some n-heptane. The filter cake was dried under vacuum before being dissolved in acetonitrile and heated to 35° C. jacket temperature. n-Heptane was added followed by a suspension of product seed material, which consists of one part (IB). HCl and 33 parts n-heptane. Additional n-heptane was added and the resulting suspension was cooled to −1° C. jacket temperature. Stirring was continued for 18 h. Afterwards, the resulting suspension was filtered and the filter cake was washed with n-heptane. The filter cake was dried under vacuum at room temperature to afford the desired product, i.e. crystallized (IB).

HCl (6.1 g, 61% yield, e.r. 99:1). ¹H NMR (DMSO-d₆): 8.32 (br s, NH₃⁺), 6.84 (br s, OH), 2.99 (s, 2H), 1.39 (s, 3H). ¹³C NMR (DMSO-d₆): 125.8 (CF₃), 70.4 (C—CF₃), 42.2 (CH₂), 18.6 (CH₃).

### Flow Procedure

A stainless steel cartridge having an internal diameter of 9.5 mm and a length of 90 mm, was packed with a mixture of water-damp sponge-nickel (14.5 g) and inert silicon carbide (9.58 g) which had been previously thoroughly mixed. The cartridge is fitted with liquid and gas inlets, and contains an 5-μm frit preventing solid materials from leaving the cartridge at the outlet. At start-up the catalyst-loaded cartridge was placed in a water-ice bath and flushed with a 1:4 v/v mixture of methanol and isopropyl acetate for not less than 12 column volumes. The liquid stream was then switched to a 0.25 M solution of (IA) in a 1:4 v/v mixture of methanol and isopropyl acetate. The feed was also pre-cooled in an ice bath to 0 to 5 degrees Celsius. The feed was supplied to the cartridge at a flow rate of 3 mL/min by a gear-pump. Hydrogen gas was generated by electrolysis of water, and supplied at a flow rate of 99.0 mL/min by a mass-flow controller, and the entire setup was pressurized to 10 bar using a back-pressure regulator. Accounting for the presence of hydrogen gas a superficial residence time of 31 seconds was calculated. Each pass through the cartridge provides a conversion to product of between 50 and 60 A %, upon which the solution containing product was added back into the feed bottle and passed again through the cartridge until a total of 3 passes had taken place.

For an experiment with a 180 minute duration, a total of 17.92 g of starting material was employed in a volume of 500 mL solvent (4.0 m %). Conversion to product after 3 passes was 98 A %, and upon work-up of liquid-fractions to the hydrochloric acid salt of the product (according to the standard crystallization method employing HCl/methanol as described previously), an isolated yield of 75% was observed with a quality of 99 A % HPLC.

### Example 18

### Coupling Process to Synthesise Compound (IC)

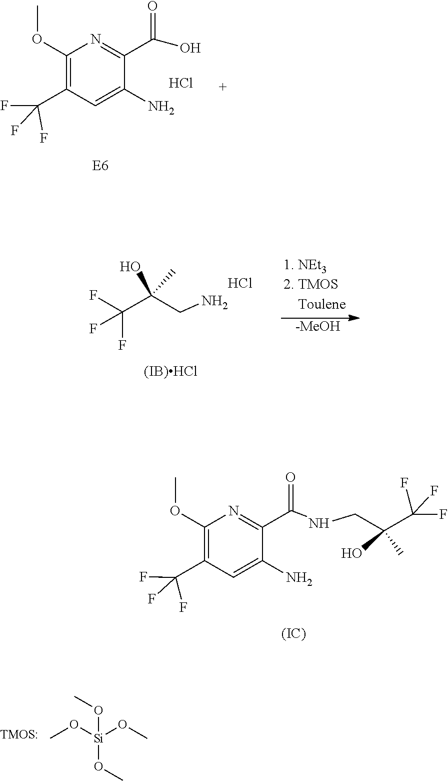

E6 (41.4 g, 152 mMol) and crystallized (IB). HCl (30 g, 167 mMol) were charged to a reactor followed by 410 ml of toluene. Under stirring, triethylamine (38 g, 380 mMol) followed by tetramethyl orthosilicate (TMOS) (46 g, 304 mMol) were added. The reaction mixture was heated up to 110° C. and stirred for 3 to 16 h while methanol, formed by the reaction, is distilled off slowly. Completion of the reaction was monitored by HPLC. An aqueous NaOH solution (330 ml, 2 molar) was added and the mixture was stirred for 2 h at 50° C. The aqueous phase was separated and discarded. At 50° C. aqueous HCl (330 ml, 10%) was added and the aqueous phase discarded. After a water wash, the organic solution was dried and concentrated by distillation. (IC) was crystallized by addition of 240 ml of n-heptane and cooling to 0° C. After drying at 40° C. under vacuum the compound (IC) was obtained in 80% yield (44 g). ¹H-NMR (DMSO-d₆): δ ppm 8.29 (m, 1H), 7.67 (s, 1H), 6.68 (br, 2H), 6.28 (s, 1H), 3.92 (s, 3H), 3.69-3.43 (m, 2H), 1.25 (s, 3H). ¹³C-NMR (DMSO-d₆): δ ppm 18.95, 42.2, 53.5, 72.6-71.9 (m), 116.5-115.6 (m), 124.9-121.0 (2C, m), 126.3, 128.3 (m), 140.9, 148.5, 166.3.

### Example 19

Round 14 Evolution and Screening of Engineered Polypeptides Derived from SEQ ID NO: 606 for Improved Production of Compound (IA)

The polynucleotide from example 12 SEQ ID NO: 413 encoding the most active polypeptide with oxynitrilase activity of SEQ ID NO: 414 was used to generate the engineered polypeptides of Table 19-1. These polypeptides displayed improved oxynitrilase activity under the desired conditions e.g., higher thermostability and the improvement in the formation of the nitro alcohol compound (IA), that was produced in situ from the substrates trifluoroacetone and nitromethane, compounds (1) and (2), respectively, as compared to the starting polypeptide. Some polypeptides displayed improved product formation of nitro alcohol product (IA) compared to the starting polypeptide are noted in Table 19-1. The engineered polypeptides, having the amino acid sequences of even-numbered sequence identifiers were generated from the “backbone” amino acid sequence of SEQ ID NO: 414, as described below together with the analytical method described herein.

Directed evolution began with the polynucleotide set forth in SEQ ID NO: 413. Libraries of engineered polypeptides were generated using various well-known techniques (e.g., saturation mutagenesis, recombination of previously identified beneficial amino acid differences) and screened using HTP assay and analysis methods, described below, that measured the polypeptides' ability to produce compound (IA).

The enzyme assays were carried out in 96-well deep-well (1.1 mL total volume) plates, in 100 μL total reaction volume per well. The reactions contained 5 v/v % of undiluted oxynitrilase lysate, prepared as described in Example 3 and had then been either preincubated for 1 h at 4° C. (noted with “no thermoincubation” in Tab. 19-1) or 52° C. (noted with “thermoincubation” in Tab. 19-1). All the other conditions did not vary to the previous round of evolution: 0.75 M trifluoroacetone, compound (1), 0.9 M nitromethane, compound (2), 30% isopropyl acetate in citrate buffer at pH 6.0. The reaction plates were heat-sealed and shaken at 150 rpm and 22° C.

After overnight incubation (˜22 hours), 650 L/well of MeOH was added to the reaction plates. The quenched reaction plates were sealed, mixed well, and centrifuged at 4,000 rpm for 15 mnt.

Subsequently 15 μL/well of the quenched reaction plate was removed and added to a shallow well 96-well plate containing 185 μL of MeOH. The plates were sealed and mixed well. These samples were then analyzed by TIPLC to determine the activity of the enzyme variants using the analytical method described in Table 5-1. Selected oxynitrilase variants showing a faster product formation of (IA), relative to SEQ ID NO: 414 are shown in Table 19-1. The high S-selectivity of >9500 (>9000 ee) towards the (IA) product of the most active polypeptides SEQ ID NO: 610 and 620 was confirmed by chiral GC analysis using the analytical method described in Table 5-3.

TABLE 19-1
Oxynitrilase Activity for the Production of
Compound (IA) Relative to SEQ ID NO: 414

| | | FIOP percent | |
|---|---|---|---|
| | | conversion | FIOP percent |
| | | Mean - no | conversion |
| | | thermo- | Mean - thermo- |
| SEQ | | incubation | incubation |
| ID NO: | Amino Acid Differences | (Relative to SEQ | (Relative to SEQ |
| (nt/aa) | (Relative to SEQ ID NO: 414) | ID NO: 414)¹ | ID NO: 414)² |
| 607/608 | K221I | +++ | − |
| 609/610 | A63Q/V123T | +++ | − |
| 611/612 | K221D | ++ | − |
| 613/614 | R170S | ++ | ++ |
| 615/616 | E249K | ++ | − |
| 617/618 | V123T | + | + |
| 619/620 | E190A | +++ | ++ |
| 621/622 | S133F | + | − |
| 623/624 | G27S/D182K | ++ | − |
| 625/626 | R170L | + | + |
| 627/628 | L17V/S253C/S255V | + | + |
| 629/630 | K221G | ++ | − |
| 631/632 | S194G | + | − |
| 633/634 | K221V | + | − |
| 635/636 | S66D | + | ++ |
| 637/638 | R196P | + | + |
| 639/640 | R210K | + | + |

¹Levels of increased activity were determined relative to the reference polypeptide of SEQ ID NO: 414 and defined as follows:
“+” .99 to 1.13 (first 50%),
“++” > 1.13 (next 30%),
“+++” > 1.16 (top 20%)
²Levels of increased activity were determined relative to the reference polypeptide of SEQ ID NO: 414 and defined as follows:
“−” .45 to .98 (first 50%),
“+” > .98 (next 30%),
“++” > 1.16 (top 20%)

### Example 20

### Substrate Scope of Evolved Oxynitrilases

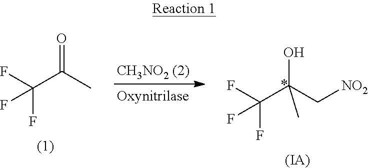

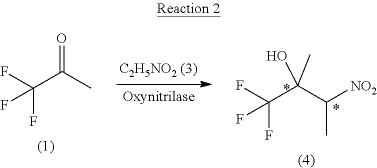

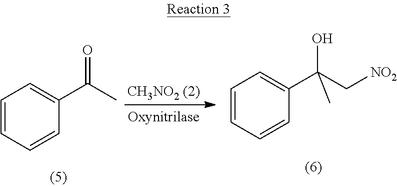

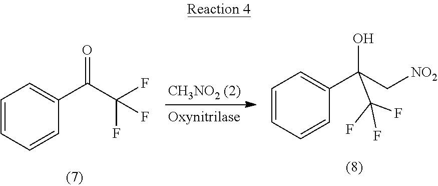 [SMILES: O=C(c1ccccc1)C(F)(F)F ; O=[N+]([O-])CC(O)(c1ccccc1)C(F)(F)F]

From different rounds of evolution the most active polypeptide with oxynitrilase activity, SEQ ID NO: 2, 156, 414 and 606 were produced as shake flask powder (SFP) and tested for their oxynitrilase activity on different substrate mixtures. Reaction 1 was the reference reaction with trifluoroacetone (1) and nitromethane (2) as substrates to form the nitro alcohol compound (IA). In reaction 2 the oxynitrilases were incubated with trifluoroacetone (1) and nitroethane (3) to investigate formation of 1,1,1-trifluoro-2-methyl-3-nitrobutan-2-ol (4). In reaction 3 acetophenone (5) and nitromethane (2) were tested as substrates to investigate formation of the product 1-nitro-2-phenylpropan-2-ol (6) and in reaction 4 2,2,2-trifluoro-1-phenylethan-1-one (7) and nitromethane (2) were tested as substrates to investigate formation of 1,1,1-trifluoro-3-nitro-2-phenylpropan-2-ol (8).

In a first experimental setup the oxynitrilase variants were investigated for their activity in regards of reaction 1, 2 and 3. The enzyme assays were carried out in 96-well deep-well plates (2 mL total volume), in 400 μL total reaction volume per well. The reaction mixture contained 0.1 M sodium citrate buffer pH 6.0, 10vol % iPrOAc and in reaction 1:1 g/L (9 mM) trifluoroacetone (1) and 11 g/L (180 mM) nitromethane (2); in reaction 2:1 g/L (9 mM) trifluoroacetone (1) and 13.5 g/L (180 mM) nitroethane (3); and in reaction 3:1.1 g/L (9 mM) acetophenone (5) and 11 g/L (180 mM) nitromethane (2). The reaction plates were heat-sealed and shaken at 200 rpm and 22° C.

After overnight incubation (˜22 hours), 1.2 ml/well of MeOH was added to the reaction plates. The quenched reaction plates were sealed, mixed well, and centrifuged at 4,000 rpm for 10 min. Subsequently 200 μL/well of the quenched reaction was transferred to a shallow well 96-well plate for HPLC-MS analytics to determine the activity of the enzyme variants using the analytical method described in Table 5.4. The relative product peak areas observed for the reaction setup 1,2 and 3 are shown in Table 20-1. The masses of the products (IA) and (4) could be confirmed via LC-MS. In the reaction 3 no product formation of (6) was observed in LC-MS using the analytical method described in Table 5.4 and GC-MS/FID using the analytical method described in Table 5.5 indicating no enzyme activity when acetophenone was used as substrate. The results of reaction 2 show the increased substrate acceptance of the evolved oxynitrilases for nitroethane (3), whereas no activity had been detected for the wildtype enzyme SEQ ID 606. After initial activity detected for SEQ ID 2, a >25-fold increase in the enzymatic productivity was achieved over the subsequent rounds of evolution, as can be seen when comparing 100 g/L of the less-evolved SEQ ID 2 resulted in lower product formation than 4 g/L of SEQ ID 414.

The enzyme assays to obtain the results shown in Table 20-1-Reaction 4 were carried out in 96-well deep-well plates (1.1 mL total volume) in 200 μL total reaction volume per well. The reaction mixture contained 0.1 M sodium citrate buffer pH 6.0, 20 vol % iPrOAc and 5.2 g/L (30 mM) 2,2,2-trifluoro-1-phenylethan-1-one (7) and 36.6 g/L (600 mM) nitromethane (2). The reaction plates were heat-sealed and shaken at 200 rpm and 22° C.

After overnight incubation (˜22 hours), 0.6 mi/well of MeOH was added to the reaction plates. The quenched reaction plates were sealed, mixed well, and centrifuged at 4,000 rpm for 10 min. Subsequently 100 μL/well of the quenched reaction was transferred to a shallow well 96-well plate containing 100 μL MeOH/well. Sample were then analyzed by LC-MS analytics to determine the activity of the enzyme variants using the analytical method described in Table 5.4. The results of reaction 4 show the increased conversion to the product (8) for the evolved oxynitrilases. The most evolved oxynitrilase with the SEQ ID 414 showed highest product formation across all tested substrate combinations.

TABLE 20-1
Peak areas of the products (IA), (4) and (6) depending
on the applied enzyme SEQ ID 2, 156, 414 or 606.

| | Reaction 1 | | | | Reaction 2 | | | |
|---|---|---|---|---|---|---|---|---|
| | (1) + (2) → (IA) | | | | (1) + (3) → (4) | | | |
| | SEQ | SEQ | SEQ | SEQ | SEQ | SEQ | SEQ | SEQ |
| | ID: | ID: | ID: | ID: | ID: | ID: | ID: | ID: |
| | 606 | 2 | 156 | 414 | 606 | 2 | 156 | 414 |
| 0 g/L enzyme | 0.087 | 0.087 | 0.083 | 0.080 | — | — | — | — |
| 0.4 g/L enzyme | 0.088 | 0.121 | 0.206 | 0.231 | — | — | — | — |
| 1.2 g/L enzyme | 0.097 | 0.201 | 0.401 | 0.486 | — | — | — | — |
| 4 g/L enzyme | 0.116 | 0.404 | 0.765 | 0.908 | — | — | — | 0.100 |
| 11 g/L enzyme | 0.136 | 0.604 | 0.948 | 1.333 | — | — | 0.099 | 0.153 |
| 33 g/L enzyme | 0.197 | 0.890 | 1.282 | 1.637 | — | — | 0.154 | 0.175 |
| 100 g/L enzyme | 0.316 | 1.103 | 1.603 | 1.764 | — | 0.075 | 0.184 | 0.215 |
| | Reaction 3 | | | | Reaction 4 | | | |
| | (5) + (2) → (6) | | | | (7) + (2) → (8) | | | |
| | SEQ | SEQ | SEQ | SEQ | SEQ | SEQ | SEQ | SEQ |
| | ID: | ID: | ID: | ID: | ID: | ID: | ID: | ID: |
| | 606 | 2 | 156 | 414 | 606 | 2 | 156 | 414 |
| 0 g/L enzyme | — | — | — | — | 0.541 | 0.525 | 0.527 | 0.525 |
| 0.4 g/L enzyme | — | — | — | — | 0.678 | 0.935 | 2.158 | 5.398 |
| 1.2 g/L enzyme | — | — | — | — | 0.756 | 1.109 | 2.770 | 5.836 |
| 4 g/L enzyme | — | — | — | — | 0.888 | 1.310 | 3.297 | 6.053 |
| 11 g/L enzyme | — | — | — | — | 1.034 | 1.458 | 3.676 | 6.283 |
| 33 g/L enzyme | — | — | — | — | 1.137 | 1.562 | 4.011 | 6.685 |
| 100 g/L enzyme | — | — | — | — | 1.319 | 1.519 | 4.193 | 6.855 |

1. An engineered oxynitrilase polypeptide, which is a polypeptide of (a) or (b) below:
(a) a polypeptide comprising an amino acid sequence selected from the group consisting of SEQ ID NOs: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 608, 610, 612, 614, 616, 618, 620, 622, 624, 626, 628, 630, 632, 634, 636, 638, and 640;
or
(b) a polypeptide having oxynitrilase activity, which comprises an amino acid sequence having (i) at least 80% sequence identity to one of the polypeptides recited in (a), and (ii) a substitution, deletion, addition or insertion of one or more amino acid residues relative to said one amino acid sequence recited in (a).

2. An engineered oxynitrilase polypeptide, which is capable of coupling 1,1,1-trifluoropropan-2-one with nitromethane to produce (S)-1,1,1-trifluoro-2-methyl-3-nitropropan-2-ol under suitable reaction conditions at greater stereoselectivity and/or activity than that of SEQ ID NO: 606.

3. An engineered oxynitrilase polypeptide comprising an amino acid sequence having at least 80% sequence identity to SEQ ID NO: 606, which is, under suitable reaction conditions, capable of coupling 1,1,1-trifluoropropan-2-one with nitromethane to produce (S)-1,1,1-trifluoro-2-methyl-3-nitropropan-2-ol in an enantiomeric excess of at least 60%, 70%, 80%, 85%, 90%, 91%, 92%, 93%, 94%, 95%, 96%, 97%, 98%, 99% or more.

4. The oxynitrilase polypeptide of claim 3, wherein the suitable reaction conditions include about 5 g/L to about 150 g/L 1,1,1-trifluoropropan-2-one, nitromethane loading about 2 times the molar amount of 1,1,1-trifluoropropan-2-one, at least 3 g/L oxynitrilase polypeptide, isopropyl acetate concentration of about 20% (v/v) to about 60% (v/v), pH of about 4.0 to 8.0, and temperature of about 10° C. to 30° C.

5. The oxynitrilase polypeptide of claim 3 or 4, wherein the amino acid sequence of the oxynitrilase comprises an amino acid sequence that differs from the sequence of SEQ ID NO: 2 in one or more amino acid residues selected from: 2, 12, 28, 29, 32, 39, 50, 55, 64, 105, 111, 147, 152, 154, 160, 185, 196, 203, 208, 209, 232, 233, and 250, wherein the numbering refers to SEQ ID NO: 2, and wherein the polypeptide has oxynitrilase activity.

6. The oxynitrilase polypeptide of claim 5, wherein the amino acid sequence of the oxynitrilase comprises one or more of the following amino acid residues:
X2 is absent; X12 is I; X28 is G; X29 is W; X32 is T; X39 is F or V; X50 is E or D; X55 is G;
X64 is A; X105 is G; X111 is S; X147 is K; X152 is L; X154 is absent; X160 is M; X185 is R;
X196 is G; X203 is C; X208 is R or S; X209 is V; X232 is G; X233 is G; or X250 is G; wherein the numbering refers to SEQ ID NO: 2.

7. The oxynitrilase polypeptide of claim 3 to 6, wherein the amino acid sequence of the oxynitrilase comprises an amino acid sequence that differs from the sequence of SEQ ID NO: 2 in one or more amino acid residues selected from: 39, 105, and 154, wherein the numbering refers to SEQ ID NO: 2, and wherein the polypeptide has oxynitrilase activity.

8. The oxynitrilase polypeptide of claim 7, wherein the amino acid sequence of the oxynitrilase comprises one or more of the following amino acid residues:
X39 is F or V; X105 is G; or X154 is absent; wherein the numbering refers to SEQ ID NO: 2.

9. The oxynitrilase polypeptide according to any one of claims 3 and 4, wherein the amino acid sequence of the oxynitrilase comprises an amino acid sequence that differs from the sequence of SEQ ID NO: 606 in one or more amino acid residues selected from: 2, 11, 12, 28, 29, 32, 33, 39, 43, 44, 46, 50, 55, 64, 80, 103, 105, 111, 118, 121, 147, 152, 154, 160, 172, 180, 185, 196, 203, 208, 209, 232, 233, 238, 241, 250, and 263, wherein the numbering refers to SEQ ID NO: 606, and wherein the polypeptide has oxynitrilase activity.

10. The oxynitrilase polypeptide of claim 9, wherein the amino acid sequence of the oxynitrilase comprises one or more of the following amino acid residues:
X2 is absent; X1I is S; X12 is V; X28 is G; X29 is W; X32 is T; X33 is V; X39 is F, X43 is S;
X44 is N; X46 is H; X50 is E or D; X55 is R or G; X64 is A; X80 is A; X103 is V; X105 is G;
X111 is S; X118 is V; X121 is Y; X147 is K; X152 is L; X154 is absent; X160 is M; X172 is R;
X180 is L; X185 is R; X196 is G; X203 is C; X208 is R or S; X209 is V; X232 is G; X233 is G;
X238 is M; X241 is R; X250 is G; or X263 is S; wherein the numbering refers to SEQ ID NO: 606.

11. The oxynitrilase polypeptide according to any one of claims 3, 4 and 9, wherein the amino acid sequence of the oxynitrilase comprises an amino acid sequence that differs from the sequence of SEQ ID NO: 606 in one or more amino acid residues selected from: 2, 105, 111, 154, 160, 185, 209, 232, and 250, wherein the numbering refers to SEQ ID NO: 606, and
wherein the polypeptide has oxynitrilase activity.

12. The oxynitrilase polypeptide of claim 11, wherein the amino acid sequence of the oxynitrilase comprises one or more of the following amino acid residues:
X2 is absent; X105 is G; X111 is S; X154 is absent; X160 is M; X185 is R; X209 is V; X232 is G; or
X250 is G; wherein the numbering refers to SEQ ID NO: 606.

13. A polypeptide immobilized on a solid material by chemical bond or a physical adsorption method, wherein the polypeptide is selected from the oxynitrilase polypeptides according to any one of claims 1 to 12.

14. A polynucleotide encoding the polypeptide of any one of claims 1 to 13.

15. The polynucleotide of claim 14, wherein the polynucleotide sequence is SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 607, 609, 611, 613, 615, 617, 619, 621, 623, 625, 627, 629, 631, 633, 635, 637 or 639.

16. An expression vector comprising the polynucleotide according to any one of claims 14 and 15.

17. The expression vector of claim 16, which comprises a plasmid, a cosmid, a bacteriophage or a viral vector.

18. A host cell comprising the expression vector of any one of claims 16 and 17, wherein the host cell is preferably E. coli.

19. A method of preparing an oxynitrilase polypeptide, which comprises the steps of culturing the host cell according to claim 18 and obtaining an oxynitrilase polypeptide from the culture.

20. An oxynitrilase catalyst obtainable by culturing the host cells according to claim 18, or according to the method of claim 19, wherein said oxynitrilase catalyst comprises cells or culture fluid containing the oxynitrilase polypeptides, or an article processed therewith, wherein the article refers to an extract obtained from the culture of transformant cell, an isolated product obtained by isolating or purifying an oxynitrilase from the extract, or an immobilized product obtained by immobilizing transformant cell, an extract thereof, or isolated product of the extract.

21. A process for the asymmetric synthesis of a j-nitro alcohol, the process comprising the step of contacting a nitroalkane and an aldehyde or ketone substrate with the oxynitrilase polypeptide according to any one of claims 1 to 13, to obtain a j-nitro alcohol product.

22. The process according to claim 21, wherein the aldehyde or ketone substrate comprises an electron withdrawing substituent.

23. The process according to any one of claims 21 and 22, wherein the resulting β-nitro alcohol has the structure shown in formula (I): 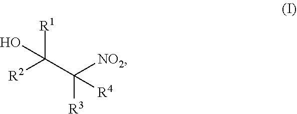
wherein:
R¹ and R² are each independently selected from H, alkyl, e.g., C₁-C₂₀alkyl, alkenyl, e.g., C₂-C₂₀alkenyl, alkynyl, e.g., C₂-C₂₀alkynyl, cycloalkyl, e.g., C₃-C₁₀cycloalkyl, aryl, e.g., C₆-C₁₄aryl, arylalkyl, e.g., C₇-C₂₀arylalkyl, heterocyclyl, e.g., 3-14 membered heterocyclyl, and heteroaryl, e.g., 5-20 membered heteroaryl,
wherein the alkyl, alkenyl, and alkynyl are each optionally substituted by one or more R^{a}, e.g., one to six R^{a},
wherein the cycloalkyl, aryl, arylalkyl, heterocyclyl, and heteroaryl are each optionally substituted by one or more R^{b}, e.g., one to six R^{b};
R³ and R⁴ are each independently selected from H and alkyl, e.g., C₁-C₂₀alkyl, wherein the alkyl, e.g., C₁-C₂₀alkyl, is optionally substituted by one or more R^{b}, e.g., one to six R^{b};
each R^{a} is at each occurrence independently selected from cycloalkyl, e.g., C₃-C₁₀cycloalkyl, aryl, e.g., C₆-C₁₄aryl, arylalkyl, e.g., C₇-C₂₀arylalkyl, heterocyclyl, e.g., 3-14 membered heterocyclyl, and heteroaryl, e.g., 5-20 membered heteroaryl, halogen, e.g., F, haloalkyl, e.g., C₁-C₂₀haloalkyl, e.g., —CF₃, —CN, —OR^{c}, —NR^{c}R^{c}, —(CH₂)ₙCOOR^{c}, —(CH₂)ₙ—C(═O)R^{c}, —(CH₂)ₙ—C(═O)NR^{c}R^{c};
each R^{b} is at each occurrence independently selected from halogen, e.g., F, haloalkyl, e.g., C₁-C₂₀haloalkyl, e.g., —CF₃, —CN, —NO₂, —OR^{c}, —NR^{c}R^{c}, —(CH₂)ₙCOOR^{c}, —(CH₂)ₙ—C(═O)R^{c}, —(CH₂)ₙ—C(═O)NR^{c}R^{c}, C₁-C₂₀alkyl, C₂-C₂₀alkenyl, or C₂-C₂₀alkynyl;
each R^{c} is at each occurrence independently selected from H, C₁-C₂₀alkyl, C₂-C₂₀alkenyl, and C₂-C₂₀alkynyl, each optionally substituted by one or more R^{b}, e.g., one to six R^{b}; and
n is 0, 1, 2, 3, 4, 5 or 6, e.g., 0, 1, 2 or 3;
the process comprising the step of contacting a nitroalkane of formula (II) which is R³R⁴CHNO₂ and an aldehyde or ketone substrate with the oxynitrilase polypeptide according to any one of claims 1 to 13, to obtain a β-nitro alcohol product of formula (I), the aldehyde or ketone substrate having the formula (III),

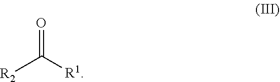

24. The process according claim 23, wherein R³ and R⁴ are each independently selected from H and C₁-C₂₀alkyl, wherein the C₁-C₂₀alkyl is optionally substituted by one to six R^{a},
wherein each R^{a} is at each occurrence independently selected from C₃-C₁₀cycloalkyl, C₆-C₁₄aryl, 3-14 membered heterocyclyl, 5-20 membered heteroaryl, halogen, e.g., F, haloalkyl, e.g., C₁-C₂₀haloalkyl, e.g., —CF₃, —OR^{c}, and —NR^{c}R^{c};
wherein each R^{c} is at each occurrence independently selected from H, C₁-C₂₀alkyl, C₂-C₂₀alkenyl, and C₂-C₂₀alkynyl.

25. The process according to any one of claims 23 and 24, wherein R³ and R⁴ are each independently selected from H and C₁-C₂₀alkyl, wherein the C₁-C₂₀alkyl is optionally substituted by one to six R^{a},
wherein each R^{a} is at each occurrence independently selected from halogen, e.g., F, C₁-C₂₀haloalkyl, e.g., —CF₃, —OR^{c}, and —NR^{c}R^{c};
wherein each R^{c} is at each occurrence independently selected from H, C₁-C₂₀alkyl, C₂-C₂₀alkenyl, and C₂-C₂₀alkynyl.

26. The process according to any one of claims 23 to 25, wherein R³ and R⁴ are each independently selected from H and C₁-C₂₀alkyl, wherein the C₁-C₂₀alkyl is optionally substituted by one to six R^{a},
wherein each R^{a} is at each occurrence independently selected from halogen, e.g., F, C₁-C₂₀haloalkyl, e.g., —CF₃, —OR^{c}, and —NR^{c}R^{c};
wherein each R^{c} is at each occurrence independently selected from H, and C₁-C₂₀alkyl.

27. The process according to any one of claims 21 to 26, wherein the resulting j-nitro alcohol has the structure shown in formula (I-i): 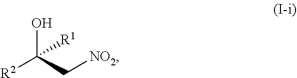
wherein:
R¹ and R² are each independently selected from H, alkyl, e.g., C₁-C₂₀alkyl, alkenyl, e.g., C₂-C₂₀alkenyl, alkynyl, e.g., C₂-C₂₀alkynyl, cycloalkyl, e.g., C₃-C₁₀cycloalkyl, aryl, e.g., C₆-C₁₄aryl, arylalkyl, e.g., C₇-C₂₀arylalkyl, heterocyclyl, e.g., 3-14 membered heterocyclyl, and heteroaryl, e.g., 5-20 membered heteroaryl,
wherein the alkyl, alkenyl, and alkynyl are each optionally substituted by one or more R^{a}, e.g., one to six R^{a},
wherein the cycloalkyl, aryl, arylalkyl, heterocyclyl, and heteroaryl are each optionally substituted by one or more R^{b}, e.g., one to six R^{b};
each R^{a} is at each occurrence independently selected from cycloalkyl, e.g., C₃-C₁₀cycloalkyl, aryl, e.g., C₆-C₁₄aryl, arylalkyl, e.g., C₇-C₂₀arylalkyl, heterocyclyl, e.g., 3-14 membered heterocyclyl, and heteroaryl, e.g., 5-20 membered heteroaryl, halogen, e.g., F, haloalkyl, e.g., C₁-C₂₀haloalkyl, e.g., —CF₃, —CN, —OR^{c}, —NR^{c}R^{c}, —(CH₂)ₙCOOR^{c}, —(CH₂)ₙ—C(═O)R^{c}, —(CH₂)ₙ—C(═O)NR^{c}R^{c};
each R^{b} is at each occurrence independently selected from halogen, e.g., F, haloalkyl, e.g., C₁-C₂₀haloalkyl, e.g., —CF₃, —CN, —NO₂, —OR^{c}, —NR^{c}R^{c}, —(CH₂)ₙCOOR^{c}, —(CH₂)ₙ—C(═O)R^{c}, —(CH₂)ₙ—C(═O)NR^{c}R^{c}, C₁-C₂₀alkyl, C₂-C₂₀alkenyl, or C₂-C₂₀alkynyl;
each R^{c} is at each occurrence independently selected from H, C₁-C₂₀alkyl, C₂-C₂₀alkenyl, and C₂-C₂₀alkynyl, each optionally substituted by one or more R^{b}, e.g., one to six R^{b}; and
n is 0, 1, 2, 3, 4, 5 or 6, e.g., 0, 1, 2 or 3;
the process comprising the step of contacting nitromethane and an aldehyde or ketone substrate with the oxynit
rilase polypeptide according to any one of claims 1 to 13, to obtain a β-nitro alcohol product of formula (I-i), the aldehyde or ketone substrate having the formula (III),

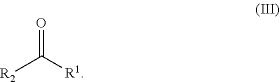

28. The process according to any one of claims 23 to 27, wherein R¹ and R² are each independently selected from H, C₁-C₂₀alkyl, C₂-C₂₀alkenyl, C₂-C₂₀alkynyl, C₃-C₁₀cycloalkyl, C₆-C₁₄aryl, C₇-C₂₀arylalkyl, 3-14 membered heterocyclyl, and 5-20 membered heteroaryl,
wherein the C₁-C₂₀alkyl, C₂-C₂₀alkenyl, and C₂-C₂₀alkynyl are each optionally substituted by one to six R^{a},
wherein the C₃-C₁₀cycloalkyl, C₆-C₁₄aryl, C₇-C₂₀arylalkyl, 3-14 membered heterocyclyl, and 5-20 membered heteroaryl are each optionally substituted by one to six R^{b};
each R^{a} is at each occurrence independently selected from C₃-C₁₀cycloalkyl, C₆-C₁₄aryl, 3-14 membered heterocyclyl, 5-20 membered heteroaryl, halogen, e.g., F, haloalkyl, e.g., C₁-C₂₀haloalkyl, e.g., —CF₃, —CN, —OR^{c}, —NR^{c}R^{c}, —(CH₂)ₙCOOR^{c}, —(CH₂)ₙ—C(═O)R^{c}, —(CH₂)ₙ—C(═O)NR^{c}R^{c};
each R^{b} is at each occurrence independently selected from halogen, e.g., F, haloalkyl, e.g., C₁-C₂₀haloalkyl, e.g., —CF₃, —CN, —NO₂, —OR^{c}, —NR^{c}R^{c}, —(CH₂)ₙCOOR^{c}, —(CH₂)ₙ—C(═O)R^{c}, —(CH₂)ₙ—C(═O)NR^{c}R^{c}, C₁-C₂₀alkyl, C₂-C₂₀alkenyl, or C₂-C₂₀alkynyl;
wherein each R^{c} is at each occurrence independently selected from H, C₁-C₂₀alkyl, C₂-C₂₀alkenyl, and C₂-C₂₀alkynyl; and
n is 0, 1, 2, 3, 4, 5 or 6, e.g., 0, 1, 2 or 3.

29. The process according to any one of claims 23 to 28, wherein R¹ and R² are each independently selected from H, C₁-C₂₀alkyl, C₂-C₂₀alkenyl, C₂-C₂₀alkynyl, C₃-C₁₀cycloalkyl, C₆-C₁₄aryl, C₇-C₂₀arylalkyl, 3-14 membered heterocyclyl, and 5-20 membered heteroaryl,
wherein the C₁-C₂₀alkyl, C₂-C₂₀alkenyl, and C₂-C₂₀alkynyl are each optionally substituted by one to six R^{a},
wherein the C₃-C₁₀cycloalkyl, C₆-C₁₄aryl, C₇-C₂₀arylalkyl, 3-14 membered heterocyclyl, and 5-20 membered heteroaryl are each optionally substituted by one to six R^{b};
each R^{a} is at each occurrence independently selected from C₃-C₁₀cycloalkyl, C₆-C₁₄aryl, 3-14 membered heterocyclyl, 5-20 membered heteroaryl, halogen, e.g., F, haloalkyl, e.g., C₁-C₂₀haloalkyl, e.g., —CF₃, —CN, —OR^{c}, and —NR^{c}R^{c};
each R^{b} is at each occurrence independently selected from halogen, e.g., F, haloalkyl, e.g., C₁-C₂₀haloalkyl, e.g., —CF₃, —CN, —NO₂, —OR^{c}, —NR^{c}R^{c}, C₁-C₂₀alkyl, C₂-C₂₀alkenyl, and C₂-C₂₀alkynyl;
each R^{c} is at each occurrence independently selected from H, C₁-C₂₀alkyl, C₂-C₂₀alkenyl, and C₂-C₂₀alkynyl.

30. The process according to any one of claims 23 to 29, wherein R¹ and R² are each independently selected from H, C₁-C₂₀alkyl, C₃-C₁₀cycloalkyl, C₆-C₁₄aryl, C₇-C₂₀arylalkyl, 3-14 membered heterocyclyl, and 5-20 membered heteroaryl,
wherein the C₁-C₂₀alkyl is optionally substituted by one to six R^{a},
wherein the C₃-C₁₀cycloalkyl, C₆-C₁₄aryl, C₇-C₂₀arylalkyl, 3-14 membered heterocyclyl, and 5-20 membered heteroaryl are each optionally substituted by one to six R^{b},
each R^{a} is at each occurrence independently selected from halogen, e.g., F, C₁-C₂₀haloalkyl, e.g., —CF₃, —CN, —OR^{c}, and —NR^{c}R^{c};
each R^{b} is at each occurrence independently selected from halogen, e.g., F, haloalkyl, e.g., C₁-C₂₀haloalkyl, e.g., —CF₃, —CN, —NO₂, —OR^{c}, and —NR^{c}R^{c}; and
wherein each R^{c} is at each occurrence independently selected from H, and C₁-C₂₀alkyl.

31. The process according to any one of claims 23 to 30, wherein R¹ is selected from hydrogen, and C₁-C₂₀alkyl, wherein the C₁-C₂₀alkyl is optionally substituted by one to six R^{a},
and R² is C₁-C₂₀alkyl, wherein the C₁-C₂₀alkyl is optionally substituted by one to six R^{a},
wherein each R^{a} is at each occurrence independently selected from halogen, e.g., F, and C₁-C₂₀haloalkyl, e.g., C₁-C₂₀fluororalkyl, e.g., —CF₃.

32. The process according to any one of claims 23 to 31, wherein at least one of R¹ and R² is C₁-C₂₀fluoroalkyl, e.g., C₁-C₆fluoroalkyl.

33. The process according to any one of claims 23 to 32, wherein R¹ is selected from hydrogen, and C₁-C₆alkyl, wherein the C₁-C₆alkyl is optionally substituted by one to six F, and R² is C₁-C₆alkyl or phenyl.

34. The process according to any one of claims 23 to 33, wherein R¹ is selected from hydrogen, and trifuoromethyl, and R² is methyl or phenyl.

35. The process according to any one of claims 21 to 34, wherein the substrate is a ketone.

36. The process according to any one of claims 21 to 35, wherein the ketone substrate is

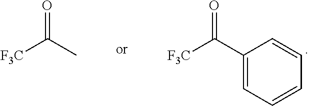

37. The process according to any one of claims 21 to 36, wherein the nitroalkane substrate is nitromethane or nitroethane.

38. A process for the asymmetric synthesis of (S)-1,1,1-trifluoro-2-methyl-3-nitropropan-2-ol (IA): 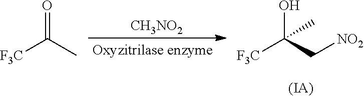
the process comprising the step of contacting nitromethane and 1,1,1-trifluoropropan-2-one with the oxynitrilase polypeptide according to any one of claims 1 to 13, to obtain (S)-1,1,1-trifluoro-2-methyl-3-nitropropan-2-ol of formula (IA).

39. The process according to any one of claims 21 to 38, wherein the β-nitro alcohol is present in an enantiomeric excess of at least 60%, 70%, 80%, 85%, 90%, 91%, 92%, 93%, 94%, 95%, 96%, 97%, 98%, 99%, or more.

40. The process according to any one of claims 21 to 39, wherein the reaction is carried out in a solvent.

41. The process according to claim 40, wherein the solvent is selected from a polar solvent, non-polar solvent and ionic liquid.

42. The process according to any one of claims 21 to 41, wherein the reaction is carried out in a solvent, wherein the solvent is selected from water, methanol, ethanol, n-propanol, isopropanol, isopropyl acetate, dimethyl sulfoxide, dimethylformamide, ethyl acetate, butyl acetate, 1-octanol, hexane, heptane, octane, methyl tert-butyl ether, toluene, 1-ethyl-4-methylimidazolium tetrafluoroborate, 1-butyl-3-methylimidazolium tetrafluoroborate, 1-butyl-3-methylimidazolium hexafluorophosphate, glycerol, and polyethylene glycol.

43. The process according to any one of claims 40 to 42, wherein the reaction is carried out in the presence of a co-solvent.

44. The process according to claim 43, wherein the co-solvent is selected from dimethylsulfoxide (DMSO), and an alcohol, e.g., methanol, ethanol, n-propanol, isopropanol.

45. The process according to any one of claims 21 to 44, wherein the reaction is carried out at a temperature of 10 to 30° C.

46. The process according to any one of claims 21 to 45, wherein the reaction is carried out at a pH of 4.0 to 8.0.

47. The process according to any one of claims 21 to 46, wherein the aldehyde or ketone substrate is present at a loading of 5 g/L to 150 g/L.

48. The process according to any one of claims 21 to 47, wherein the nitroalkane is present at a loading of 5 g/L to 150 g/L.

49. The process according to any one of claims 21 to 48, wherein the nitroalkane is present in an excess of 2 stoichiometric equivalents relative to the aldehyde or ketone substrate.

50. The process according to any one of claims 21 to 49, wherein the oxynitrilase polypeptide is present at a concentration of at least 3 g/L enzyme.

51. The process according to any one of claims 21 to 50, wherein the solvent is present at a concentration of 0 to 500 g/L, e.g., 100 to 200 g/L, e.g., 196 g/L.

52. A process for synthesizing (S)-3-amino-1,1,1-trifluoro-2-methylpropan-2-ol of formula (IB): 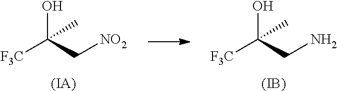
the process comprising the step of contacting (IA) with hydrogen under suitable hydrogenation conditions, to obtain (S)-3-amino-1,1,1-trifluoro-2-methylpropan-2-ol (IB), wherein (IA) is synthesized by the process according to any one of claims 21 to 51.

53. The process according to claim 52, wherein the reaction is carried out in the presence of a catalyst, optionally wherein the catalyst is selected from Raney nickel, Raney cobalt, Pd/C and Pt/C.

54. The process according to claim 53, wherein the catalyst is present at a loading of at least 2 wt. %, e.g., at least 5 wt. %, at least 10 wt. %.

55. The process according to any one of claims 52 to 54, wherein the reaction is carried out in the presence of activated charcoal.

56. The process according to any one of claims 52 to 55, wherein the reaction is carried out in a solvent, wherein the solvent is selected from water, methanol, ethanol, propanol, isopropanol, ethyl acetate, isopropyl acetate, tert-butyl methyl ether.

57. The process according to any one of claims 52 to 56, wherein the reaction is carried out at a temperature of 20 to 60° C., e.g., 20, 30, 40, 50° C.

58. The process according to any one of claims 52 to 57, wherein the compound of formula (IA) is present at a concentration of at least 2 wt. %, e.g., 2, 4, 8, 10 wt. %.

59. The process according to any one of claims 52 to 58, wherein the process is carried out in batch or flow.

60. The process according to any one of claims 52 to 59, further comprising the step of converting the compound of formula (IB) to an acid salt.

61. The process according to claim 60, further comprising the step of recrystallizing the acid salt.

62. The process according to any one of claims 60 and 61, wherein the acid salt is a HCl salt.

63. A process for synthesizing (S)-3-amino-6-methoxy-N-(3,3,3-trifluoro-2-hydroxy-2-methylpropyl)-5-(trifluoromethyl)picolinamide of formula (IC) 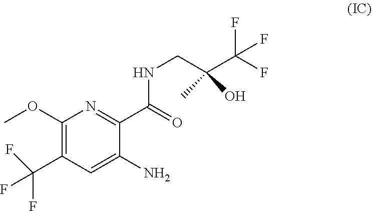 [SMILES: COc1nc(C(=O)NC[C@](C)(O)C(F)(F)F)c(N)cc1C(F)(F)F]
the process comprising the step according to claim 38.

64. The process according to claim 63, further comprising the step according to any one of claims 39 to 51.

65. The process according to any one of claims 63 and 64, further comprising the step according to claim 52.

66. The process according to claim 65, further comprising the step according to any one of claims 53 to 62.
